(19) United States
(12) Patent Application Publication
(10) Pub. No.: US 2026/0242819 A1
(43) Pub. Date: Aug. 20, 2026

(54) QUINOLINE-CONTAINING MONOMERS AND POLYMERS CONTAINING THE SAME

(71) Applicant: REGENTS OF THE UNIVERSITY OF MINNESOTA, Minneapolis, MN (US)

(72) Inventors: Theresa M. Reineke, Vadnais Heights, MN (US); Punarbasu Roy, Coochbehar (IN)

(21) Appl. No.: 18/872,144

(22) PCT Filed: Jun. 9, 2023
(86) PCT No.: PCT/US2023/024923

§ 371 Date: Dec. 5, 2024

Related U.S. Application Data
(60) Provisional application No. 63/350,525, filed on Jun. 9, 2022.

(51) Int. Cl. C12N 15/85; C08F 22/10

(52) U.S. Cl. CPC C12N 15/85; C08F 22/10

(57) ABSTRACT

Quinoline-containing monomers and polymers polymerized from at least a quinoline-containing monomer. Polyplex transfection agents comprising a quinoline containing-polymer and an oligonucleotide. Kits that include a transfection agent, the transfection agent. Methods of making a polyplex transfection complex. Methods of transfecting a cell with a polyplex transfection complex.

### CROSS-REFERENCE TO RELATED APPLICATIONS

This application claims the benefit of U.S. Provisional Patent Application No. 63/350,525, filed on Jun. 9, 2022, which is incorporated by reference herein in its entirety.

### GOVERNMENT FUNDING

This invention was made with government support under DMR-1904853 awarded by the National Science Foundation. The government has certain rights in the invention.

### SUMMARY

This disclosure describes, in one aspect, a quinoline-containing polymer polymerized from at least a quinoline-containing monomer of the structure

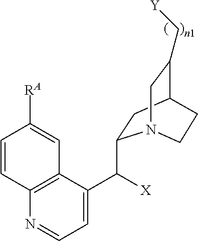

where R^{A} is H or OCH₃; n1 is an integer from 1 to 5; X is

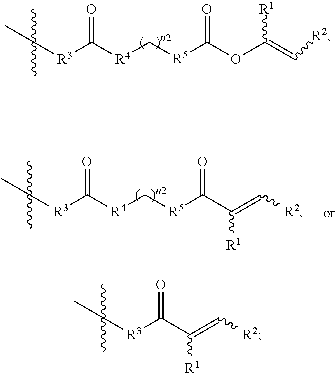

and Y is alkyl or alkenyl. R³, R⁴, and R⁵, are each independently O, NH, NR²⁰, or S. R²⁰ is methyl, ethyl, or propyl. R¹ and R² are each independently H or alkyl. n2 is an integer from 1 to 10.

As used throughout this disclosure, the term “independently” means, when used in the context of chemical substituents, that the identity of the substituent may be selected from among the listed options without regard to the identity of any other substituent.

In some embodiments, the polymer is a homopolymer polymerized from the quinoline-containing monomer. In other embodiments, the polymer is a copolymer polymerized from the quinoline-containing monomer and a second monomer.

In another aspect, this disclosure describes, a composition, such as a transfection composition, that includes a polyplex transfection complex. The polyplex transfection complex includes the quinoline-containing polymers of the present disclosure and an oligonucleotide. The composition also includes a transfection medium.

In yet another aspect, this disclosure describes a method of forming the polyplex transfection complexes of the present disclosure. The method includes mixing the polymer with the oligonucleotide to create a first mixture; and incubating the first mixture for a period of time to form a second mixture comprising the polyplex transfection complex.

In another aspect, this disclosure describes a method of transfecting a cell. The method includes contacting a cell with a transfection composition of the present disclosure to form a transfection mixture. The method further includes incubating the transfection mixture for a transfection time. In some embodiments, the method further includes quenching the transfection mixture.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows synthesis the scheme and chemical structure of a polymer synthesized from 2-hydroxyethyl acrylate (HEA) and quinine monomers. The resultant polymer is referred to as QCR.

FIG. 2A shows the synthesis scheme for the hydroquinine monomer (HQ) and copolymerization of HQ with 2-hydroxyethyl acrylate (HEA) using the controlled polymerization method of reversible addition fragmentation chain transfer (RAFT). Polymers made via this scheme are referred to as HQ-X polymers where X indicated the mol-% of the HQ monomer in the polymer.

FIG. 2B shows the annotated ¹H NMR spectrum of the hydroquinine monomer (HQ) in CDCl₃.

FIG. 2C shows the annotated ¹³C NMR spectrum of the hydroquinine monomer (HQ) in CDCl₃.

FIG. 2D shows the high resolution mass spectrum of the hydroquinine monomer (HQ).

FIG. 2E shows the annotated ¹H NMR spectrum of the HQ-25 polymer in DMSO-D₆. HQ-25 was formed via RAFT polymerization. The mole percentage of HQ in the polymer chain was calculated from the ratio of the resonance integrations of the hydroxyl group of HEA (resonance labeled 2 at 4.77 ppm; one proton for every HEA repeat unit) and the hydrogens from the quinoline ring and carbamate group of HQ (resonances labeled with Q, six protons for every HQ repeat unit).

FIG. 3A shows a schematic for the dye exclusion assay. Intercalation of PICOGREEN in pDNA results in bright green fluorescence. Binding of polymer with pDNA and the subsequent compaction leads to exclusion of PICOGREEN from the pDNA resulting in decrease in fluorescence intensity.

FIG. 3B is a plot showing the normalized fluorescence intensities from the dye exclusion assay of polyplexes formed with pDNA and HQ-X polymers or the QCR polymer. For all N/P ratios, a higher mole percentage of HQ in polymer leads to stronger binding and compaction of pDNA as indicated by gradual decrease in the fluorescence intensity.

FIG. 4A is a schematic for forming fluorescently labeled aggregated polyplexes using polymers and Cy5-labeled pDNA. The influence of the polymer monomer composition on protein adsorption on the aggregated polyplexes was studied by incubating the aggregated polyplexes with either fetal bovine serum (FBS) or ALEXAFLUOR 488 labeled bovine serum albumin (BSA-AF488).

FIG. 4B show flow cytometry scatter plots of the aggregated polyplexes formed using various HQ-X polymers. Cy5 intensity is on the Y-axis and ALEXAFLUOR 488 intensity is on the X-axis.

FIG. 4C is a plot showing the geometric mean fluorescence intensity (MFI) of Cy5 from the aggregated polyplexes formed from various HQ-X polymers and pDNA before and after incubation with FBS. Aggregated polyplexes formed with QCR have higher pDNA loading compared to aggregated polyplexes formed with HQ-12, HQ-17, and even HQ-25. While HQ-X polymers have lower loading capacity for pDNA, their polyplexes were found to be more resistant to protein-mediated payload unpackaging compared to QCR. Statistical significance was evaluated using two-way ANOVA followed by Šídák's multiple comparisons test (*** p≤0.001).

FIG. 4D is a plot showing the geometric mean fluorescence intensity (MFI) of ALEXAFLUOR 488 from the aggregated polyplexes in FIGS. 4B and 4C. HQ-X polymers show significantly lower protein adsorption compared to QCR. Statistical significance was evaluated using one-way ANOVA followed by Dunnett's multiple comparisons test (*** p≤0.001).

FIG. 5A shows representative widefield fluorescence images of HEK293T cells, 48 hours after transfection with polyplexes formed from various HQ-X polymers and pZsGreen1-N1 plasmid, N/P=16 (Scale bar=200 micrometers (μm)). Upon successful transfection, the cells produce ZsGreen1 protein that has green fluorescence.

FIG. 5B is a plot showing the transfection efficiency assessed by quantifying the percentage of live HEK293T cells that are ZsGreen1+, via flow cytometry. Data are the mean of three replicates±standard deviation. Cells transfected using polyplexes formed from HQ-44, HQ-60, and HQ-100 did not show green fluorescence even at the 96 hour time point.

FIG. 6A is a plot showing the cellular uptake of the polyplexes, measured with flow cytometry, based on the fluorescence intensity of HQ-X observed from the cells. Data are the mean of three replicates=standard deviation.

FIG. 6B is a plot showing a comparison of fluorescence intensities of HQ-X polymers in 0.05 M acetic acid in water. The amount of polymer in the solution was adjusted for each composition to have a HQ concentration of 1 mM for all polymers. At lower mole percentages of HQ, the HQ fluorescence can be used to directly quantify cellular uptake without additional fluorescent tags. However, at higher percentages, self-quenching of HQ fluorescence leads to higher number of false negatives for HQ+ live cells.

FIG. 7 is a plot showing the cellular uptake of the polyplexes formed from HQ-X polymers and Cy5 tagged pDNA, measured with flow cytometry, based on the fluorescence intensity of Cy5 observed from the cells. Data are the mean of three replicates±standard deviation.

FIG. 8 shows representative 3D images (left) and contour surface rendering of the 3D confocal images (right) of HEK293T cells, 24 hours after transfection with polyplexes formed with various HQ-X polymers and Cy5-laveled pDNA, obtained using confocal laser scanning microscopy. Cy5-labeled pDNA was used as the payload at N/P=16 (outside cytoplasm: gray, inside cytoplasm: yellow). Lysosomes (red) were stained using an anti-LAMP primary antibody and an ALEXAFLUOR 555 labeled secondary antibody. Nucleus was stained with DAPI (blue). Fluorescence of ZsGreen1 was used as the cytosolic stain (green). For untreated control, HQ-44, and HQ-60, cell cytoplasm was visualized by staining the actin filaments using ALEXAFLUOR 488 labeled Phalloidin. Pearson's correlation coefficients (PCC) for colocalization between pDNA and lysosome are mentioned at the top of the respective images. The contour surface renderings were constructed to evaluate distance between the nucleus and the Cy5-labeled pDNA present inside the cells. Scale bar is 10 micrometers.

FIGS. 9A-9G show SEC traces (light scattering: LS, differential RI: dRI) of the HQ-12 (9A), IIQ-17 (9B), IIQ-25 (9C), IIQ-35 (9D), IIQ-44 (9E), IIQ-60 (9F), and IIQ-100 (9G) polymers. Molar masses (MM) were determined with Multi Angle Light Scattering (MALS) detector using dn/dc values estimated from 100% mass recovery method.

FIGS. 10A-10I show plots of log (θ/1−θ) vs pKₐ-pH for determining the Hill coefficient (n_{Hill}), from the slope of the linear fit, for the HQ monomer (10A) and HQ-12 (10B), HQ-17 (10C), HQ-25 (10D), HQ-35 (10E), HQ-44 (10F), HQ-60 (10G), HQ-100 (10H), and QCR (10I) polymers. FIG. 10J shows the pKₐ and n_{Hill} of HQ and QCR.

FIGS. 11A-11H show light microscope images of the aggregated polyplexes (scale bar is 200 micrometers) formed with HQ-12 (11A), HQ-17 (10B), HQ-25 (10C), HQ-35 (10D), HQ-44 (10E), HQ-60 (10F), HQ-100 (10G), and QCR (10H). At lower mole percentages of HQ (<44%) in the polymer chain, the aggregated polyplex particles are smaller and appear to be spherical, similar to the case of QCR at N/P=8. For HQ-44, HQ-60, and HQ-100, the aggregates are larger and appear like flocculant particles with irregular shape. Due to their large size, they were beyond the scope of analysis using dynamic light scattering. FIG. 11I shows a representation of the two different kinds of polyplexes formed with HQ-X polymers (not to scale). HQ-X polymers have a mole percentage of HQ≤25%, the polyplex particles appear spherical. HQ-X polymers having an HQ mole percentage between 25% and 44%, the polyplexes appear irregularly shaped. HQ-X polymers have HQ mole percentages≥44% the polyplexes are also flocculant in nature.

FIG. 12 shows the hydrodynamic diameter (dh) of the polyplex particles formed from various HQ-X polymers and pDNA before and after the aggregation step (i.e., dilution), at three different N/P ratios. All polymers associated with pDNA to form polyplex particles having diameters ranging between 50 nm to 100 nm. Aggregation induced by addition of FLUOROBRITE DMEM resulted in larger particles (dh>400 nm). For HQ-44, HQ-60, and HQ-100, the polyplexes immediately aggregated into very large flocculant particles which were beyond the range of measurement for DLS.

FIG. 13 shows a schematic workflow for the flow cytometry based assay on the fluorescently labeled polyplexes.

FIGS. 14A-14D are forward (FSC) and side scattering (SSC) profiles for the aggregated polyplexes obtained from direct flow cytometric analysis for polyplexes formed with QCR, HQ-12, and HQ-17 (14A and 14B); and HQ-25 and HQ-35 (FIGS. 14C and 14D).

FIGS. 15A-15D are scatter plots from the flow cytometry assay of the polyplex particles formed with QCR, HQ-12, and HQ-17 (15A and 15B); and HQ-25 and HQ-35 (15C and 15D). The x-axis represents the intensity of the ALEXAFLUOR 488 associated with adsorbed albumin protein. The y-axis represents intensity of Cy5 that is associated with the Cy-5 labeled pDNA used to form polyplexes.

FIGS. 16A-16C show the general gating scheme for flow cytometric analysis for untreated cells (16A), LIPOFECTAMINE 2000 treated cells (15B), and HQ-25 (N/P=12) treated cells (15C). Live cells were gated using SYTOX Red dead stain marker.

FIG. 17A is a plot showing the geometric mean fluorescence intensity (MFI) of ZsGreen1 from HEK293T cells, 48 hours after transfection via a polyplex formed from various HQ-X polymers and a ZsGreem1-N1 plasmid.

FIG. 17B is a plot showing the relative cell viability of the cells 48 hours after transfection.

FIG. 18 is a plot showing the geometric mean fluorescence intensity (MFI) of ZsGreen1 from the HEK293T cells, 24, 48, and 96 hours after transfection with via a polyplex formed from various HQ-X polymers and a ZsGreen1-N1 plasmid.

FIG. 19A shows the absorption and emission spectra of 1 mM the HQ monomer in 0.05 mM H₂SO₄.

FIG. 19B is a plot showing the geometric mean fluorescence intensity (MFI) HQ from live cells, 24 hours after transfection via polyplexes formed from various HQ-X polymers and pDNA.

FIG. 20 is a plot showing the geometric mean fluorescence intensity of Cy5 from live cells, 24 hours after transfection via polyplexes formed from HQ-X polymers and Cy5-labeled pDNA.

FIGS. 21A and 21B are representative cytofluorograms generated from the images obtained from confocal microscopy imaging experiments following transfection using polyplexes formed from HQ-X polymers and Cy5 labeled pDNA. The x-axis represents intensity of ALEXAFLUOR 555 associated with lysosomes and the y-axis represents the intensity of Cy5 associated with the delivered Cy5 labeled pDNA. The cytofluorograms were used to calculate Pearson's correlation coefficient (PCC) to compare extent of colocalization of lysosomes and the pDNA payload, as a means to compare endosomal escape of the pDNA, across different samples.

FIG. 22 shows a representative 3D images (top view) of HEK293T cells, that were treated with Cy5-labeled pDNA but with any polymer, 24 hours after transfection, obtained using confocal laser scanning microscopy (left image). Cy5-labeled pDNA was used as the payload at N/P=16 (gray). Lysosomes (red) were stained using an anti-LAMP primary antibody and an ALEXAFLUOR 555 labeled secondary antibody. Nucleus was stained with DAPI (blue). Fluorescence of ZsGreen1 was used as the cytosolic stain (green). FIG. 22 also shows a contour surface rendering of the 3D confocal images were constructed to evaluate distance between nucleus and the delivered pDNA (right). The scale bar 10 micrometers.

FIG. 23A are plots showing the distribution of the distance between polyplex particles and the nuclear periphery, 24 hours after transfection, obtained with 3D confocal microscopy images of FIG. 8 and FIG. 22. FIG. 23B shows the results of comparing of the distance distributions with the Kruskal-Wallis test followed by Dunn's multiple comparisons test. Distance distribution of HQ-44 and HQ-60 are significantly different than the rest of the samples. Polyplexes formed from HQ-44 and HQ-60 have a higher average nuclear distance (>4 micrometer) than the rest of the samples (<3 micrometer).

FIG. 24 is a plot showing a comparison of the fluorescence of Cy5-labeled pDNA in the free state (pDNA) against the bound state within polyplexes formed with the HQ-X polymers. The results indicate the Cy5 fluorescence intensity does not alter significantly in the polyplex across all HQ-X polymers. The polyplexes were formed at N/P=12 by adding HQ-X polymer solution to Cy5-labeled pDNA similar to the protocol followed for the particle flow cytometry. After allowing the polymers to bind with pDNA for 30 minutes, Cy5 fluorescence intensity was measured on a plate reader using monochromator (λex=640 nm, λem=681 nm).

FIGS. 25A-25C are flow cytometry scatter plots for commercially available fluorescent particles. The flow cytometry sub-micron particle size reference kit was used on the flow cytometer to test its ability to detect particles of different size ranges. Particles down to 500 nm in diameter were detectable via flow cytometer but smaller particles were missed. The results confirm that particles above 500 nm in diameter can be studied using flow cytometer based assays. Each stock solution of the particles was diluted by 4-folds with ULTRAPURE water to reduce the concentration of particles in solution. The samples were studied with flow cytometer using the high throughput mode (4 microliters per sample). Pure water samples were placed between fluorescent particle samples to wash residual particles in the flow cytometer and to reduce the chances of cross contamination. Events were recorded using both forward scattering and FITC fluorescence (recommended by manufacturer) to distinguish particles from noise.

FIGS. 26A and 26B are plots showing a comparison of the transfection efficiency of polyplexes formed with HQ-25 and ZsGreen1DNA in the presence and absence of serum. The results indicate that transfection efficiency, both in terms of % of ZsGreen1+ cells (26A) and average brightness of the cells (26B), is reduced in the presence of serum. HQ-25 was added to pDNA at N/P=12 and the initial polyplexes were allowed to form for 30 minutes followed by addition of FLUOROBRITE DMEM in 1:2 ratio to form the aggregated polyplexes. The aggregated polyplexes were then injected directly into the serum containing media. The media was changed once 24 hours after transfection. Transfection in the absence of serum was also performed parallelly for comparison and the standard transfection protocol described in the methods section were followed.

FIGS. 27A-27C are plots showing the release of pDNA from polyplexes formed using various HQ-X polymers and pDNA at N/P ratios of 8 (27A), 12 (27B), and 16 (27C), by competitive binding of heparin, using dye exclusion assay. The difference in fluorescence intensity, which is indicative of pDNA release from polyplexes caused by heparin, was compared for HQ-X polymers. The trends in the fluorescence intensity clearly indicate that extent of pDNA-release is lower for polymers with high HQ content. A standard dye exclusion assay was first performed followed by addition of heparin solution to cause release of pDNA from the polyplexes. Reference samples (without heparin) were prepared by adding the same amount of pure water instead of heparin. The normalized fluorescence intensity from the heparin-free samples were subtracted from heparin-doped samples to compare extent of pDNA release caused by heparin.

FIG. 28A shows the general synthetic scheme for HQ-X copolymers polymerized using free radical polymerization (FRP). Polymers synthesized via this scheme are referred to as FRP-HQ-X polymers where X is the mole percent of HQ in the polymer. FIG. 28B shows the characterization data for the FRP HQ-X polymers. FIG. 28C shows the hydrodynamic diameter of polyplexes formed with FRP HQ-17 and FRP HQ25 (polymer+DNA); diluted or aggregated polyplexes formed with FRP HQ-17 and FRP HQ25 (polymer+DNA+FLUOROBRITE); and the FRP IIQ-17 and FRP IIQ25 polymers incubated without DNA (polymer | FLUOROBITE).

FIG. 29 is a plot showing the percent of live cells having a quinoline signal (+Q) 24 hours, 48 hours, and 96 hours after treatment with various transfection polyplex complexes formed with the HQ-X polymers (formed via RAFT polymerization).

FIG. 30 is a plot showing the percent of live cells displaying a Cy5+, Q+, or both 48 hours after treatment with various polyplexes formulations formed with the HQ-X series of polymers (formed via RAFT polymerization).

FIG. 31A-31B are plots showing the percent of GFP expressing cells (31A) and cell viability (31B) after cells were treated with various polyplex formulations formed with HQ-X polymers (made via RAFT polymerization) or FRP HQ-X polymers (made via FRP).

FIGS. 32A and 32B are plots showing the results of a dye exclusion assay conducted in water (W) or DMEM (D) for polyplexes made from FRP HQ-X polymers (32A) and HQ-X polymers (32B).

FIG. 33A-33C are plots showing the percent of GFP expressing ARPE-19 cells (33A), probability of transfection (33B), and normalized cell viability (33C) after ARPE-19 cells were treated with various polyplex transfection complex formed using HQ-X polymers.

### DETAILED DESCRIPTION OF ILLUSTRATIVE EMBODIMENTS

This disclosure describes quinoline-containing monomer and quinoline-containing polymers. The quinoline-containing monomers may be polymerized with or without one or more addition non-quinoline containing monomers to form quinoline-containing polymers. The quinoline-containing polymers can be used as transfection agents for introducing foreign oligonucleotide sequences into a cell. For example, the quinoline-containing polymers transfection agent may complex with an oligonucleotide to form a polyplex transfection complex. One or more cells may be treated with the polyplex transfection complex to introduce the oligonucleotide into the cell(s).

Delivery of an oligonucleotide to the intracellular space of a cell is challenging both in research settings (e.g., in vitro) and in clinical settings. With the rise in gene editing technology and potential of gene therapy, there is a need for an improved method of delivering oligonucleotide to cells. Most clinical trials have been developed around the use of recombinantly engineered viruses as the delivery vehicle or vector. Viral vectors, however, have many severe limitations to their use in clinical applications. These limitations include difficulty/limitations in the scaling of production, clinical dangers such as immunogenicity and mutagenicity, and high cost.

Polymer-based and/or lipid-based oligonucleotide delivery vehicles have emerged as a promising alternative to viral delivery vehicles due to their facile scalability, ease of manufacturing, thermal stability, and wide chemical space design optimization for a particular application. For example, several of the COVID-19 vaccines are packaged in lipid-based vehicles. Additionally, several lipid-based and polymer-based transfection reagents are commercialized and commonly used for transfection in vitro, including the cationic lipid-based reagent LIPOFECTAMINE (Invitrogen, Carlsbad, CA) and the cationic polymer (consisting of linear polyethylenimine) JETPEI (Avantor, Radnor, PA). These reagents, however, show limited efficacy (e.g., limited gene expression and/or editing) in many difficult to transfect cell types. They also can be quite toxic to target cells which can be problematic for sensitive and/or valuable cell types for clinical applications.

Quinine-containing polymers may be used as transfection agents. Quinine has several properties that make it an attractive group to include in a polymer transfection agent. For example, quinine is an FDA approved antimalarial drug, inexpensive, and naturally sourced. Quinine also has well-characterized intrinsic fluorescent properties allowing polymers that include quinine to be tracked without the use of extra dyes. For example, cells that have taken up the polymer may be traced via fluorescence. Additionally, since the fluorescence of quinine is sensitive to pH and chloride ion concentrations, the polymer may be used as a probe for tracking intracellular conditions. Furthermore, quinine is an endosomolytic agent that promotes endosomolysis of lysosomes which may allow for endosomal escape of the oligonucleotide cargo from the endosome.

In one aspect, this disclosure describes a quinoline-containing monomer. In another aspect, this disclosure describes polymers polymerized from at least a quinoline-containing monomer of the present disclosure. The quinoline-containing monomers of the present disclosure can be incorporated into a polymer, such as a homopolymer and/or a copolymer. Polymers that are formed from at least quinoline-containing monomers and that retain the quinoline moiety following polymerization, are termed quinoline-containing polymers. The homopolymer and/or copolymer may be used as a transfection agent.

As used herein, the term “polymer” refers to a homopolymer, a copolymer, and a polymer made from three or more monomers. Homopolymers are made from a single monomer. Copolymers are made from two distinct monomers. Polymers made from two or more monomers may be random polymers, block polymers, graft polymers, or branched polymers.

As used herein, the term “quinoline” refers to the compound having the chemical abstract service registry number 91-22-5 and derivatives thereof wherein one or more of the hydrogen atoms of the quinoline are replaced with a bond to a chemical moiety or with a different atom.

The quinoline-containing monomers of the present disclosure may be derived from quinine (I), quinidine (II), cinchonidine (III), cinchonine (IV), hydroquinine (V), hydroquinidine (VI), hydrocinchonidine (VII), hydrocinchonine (VIII), combinations thereof, or any stereoisomer thereof.

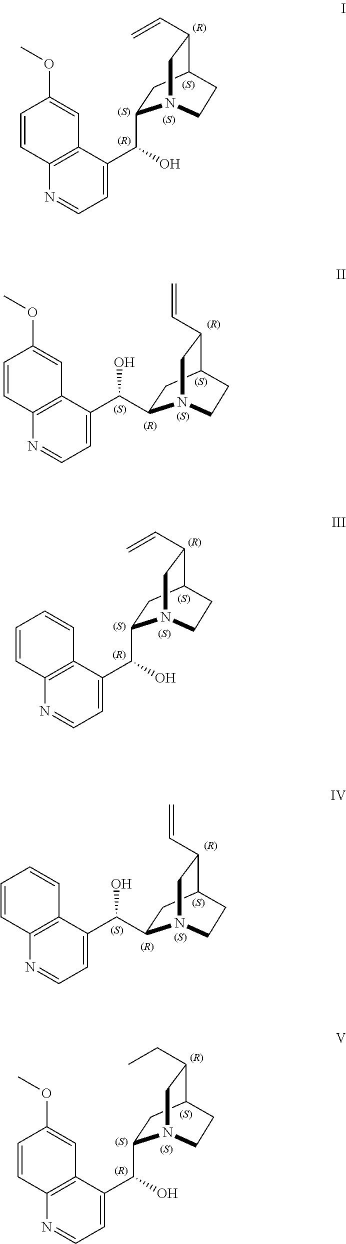

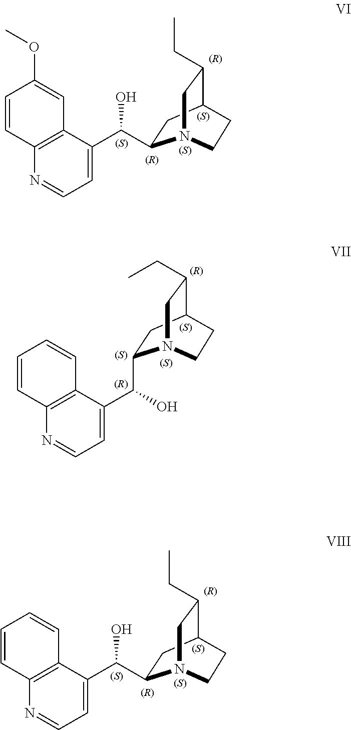

In one or more embodiments, the quinoline-containing monomer is derived from quinine. In one or more embodiments, the quinoline-containing monomer is derived from hydroquinine.

The quinoline-containing monomers of the present disclosure are of the general formula IX.

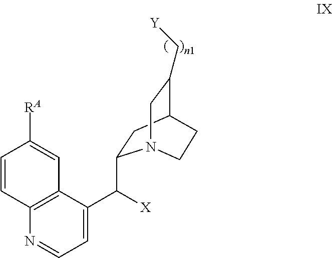

In one or more embodiments, the quinoline-containing monomers of the present disclosure are of the general formula X or XI.

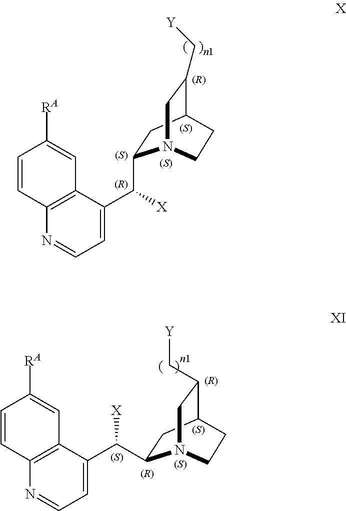

In one or more embodiments, the quinoline-containing monomer is of the general formula X. In one or more embodiments, the quinoline-containing monomer is of the general formula XI.

In formulas IX, X, and XI, R^{A} may be H or a phenyl ether. In one or more embodiments, the phenyl ether is a methyl ether (OCH₃), ethyl ether (OCH₂CH₃), n-propyl ether (OCH₂CH₂CH₃), isopropyl ether (OCH(CH₃) 2), n-butyl ether (OCH₂CH₂CH₂CH₃), isobutyl ether (OCH₂CH(CH₂)₃), or tert-butyl ether (OC(CH₃)₃). In one or more embodiments, R^{A} is methyl ether (OCH₃). In one or more embodiments, R^{A} is H.

n1 is an integer from 1 to 5. In one or more embodiments, n1 is 1. In one or more embodiments, n1 is 2. In one or more embodiments, n1 is 3. In one or more embodiments, n1 is 4. In one or more embodiments, n1 is 5.

The polymerization functional groups are located at position Y, X, or both. A polymerization functional group is a chemical moiety that is polymerizable either with itself or a different monomer. In one or more embodiments, the polymerization functional groups of the present disclosure are alkenes. Examples of polymerization functional groups that include alkenes include, but are not limited to, vinyl ethers, vinyl esters, acrylates, acrylamides, methacrylates, and methacrylamides.

X may be a group that includes a polymerization functional group. In such embodiments, X may be of the general formula XII, XIII, or XIV.

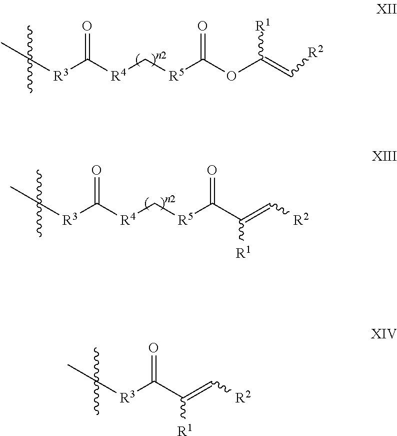

R³, R⁴, and R⁵ are each independently O, NH, NR²⁰, or S. R²⁰ is alkyl. In one or more embodiments, R²⁰ is methyl, ethyl, n-propyl, or isopropyl. In one or more embodiments, R³ is O. In one or more embodiments, R⁴ is NH. In one or more embodiments R⁵ is O.

R¹ and R² are each independently H or methyl. In one or more embodiments, R¹ is H. In one or more embodiments, R² is H. In one or more embodiments R¹ and R² are H. In one or more embodiments, R² is methyl. In one or more embodiments, R¹ is methyl. In one or more embodiments, R¹ and R² are methyl. In one or more embodiments, R¹ is methyl and R² is H. In one or more embodiments, R² is methyl and R¹ is H.

n2 is zero or an integer from 1 to 5. In one or more embodiments, n2 is 0. In one or more embodiments, n2 is 1. In one or more embodiments, n2 is 2. In one or more embodiments, n2 is 3. In one or more embodiments, n2 is 4. In one or more embodiments, n2 is 5.

X may be a group that does not include a polymerization functional group. In one or more embodiments, X is OH. In one or more embodiments, X is H.

Y may be a group that includes a polymerization functional group. In one or more embodiments Y may be a group that does not include a polymerization functional group. Y may be an alkyl, alkenyl, or of general formula XV, XVI, XVII, or XIX.

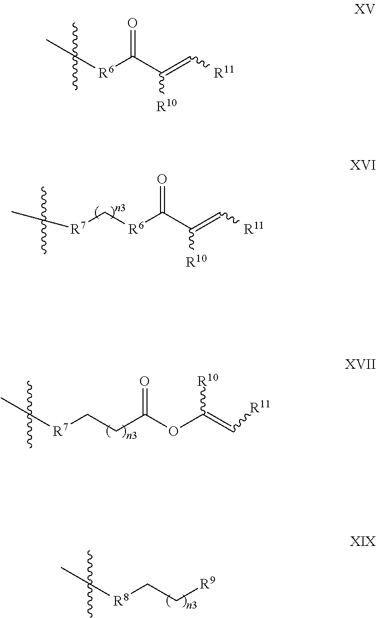

In one or more embodiments, Y is an alkyl. In some embodiments Y is a C1, C2, C3, C4, C5, C6, C7, C8, C9, or C10 alkyl. In some such embodiments Y is methyl (CH₃), ethyl (CH₂CH₃), propyl (CH₂CH₂CH₃, C(CH₃)₂), or butyl (CH₂CH₂CH₂CH₃, CH(CH₃) CH₂CH₃, CH₂CCH₃)₂).

In one or more embodiments, Y is alkenyl. In some embodiments Y is a C1, C2, C3, C4, C5, C6, C7, C8, C9, or C10 alkenyl. In one or more embodiments the alkene of the alkenyl is a terminal alkene. In one or more embodiments Y is CHCH₂.

In formulas XV, XVI, XVII, and XIX, R⁶, R⁷, R⁸ are each independently O, NH, NR²¹, or S. R²¹ is alkyl. In one or more embodiments, R²¹ is methyl, ethyl, n-propyl, or isopropyl. In one or more embodiments, R⁶ is O. In one or more embodiments, R⁶ is NH. In one or more embodiments R⁷ is O. In one or more embodiments, R⁷ is NH.

In formulas XV, XVI, XVII, and XIX, R¹⁰ and R¹¹ are each independently H or methyl. In one or more embodiments, R¹⁰ is H. In one or more embodiments, R¹¹ is H. In one or more embodiments R¹⁰ and R¹¹ are H. In one or more embodiments, R¹⁰ is methyl. In one or more embodiments, R¹¹ is methyl. In one or more embodiments, R¹⁰ and R¹¹ are methyl. In one or more embodiments, R¹⁰ is methyl and R¹¹ is H. In one or more embodiments, R¹¹ is methyl and R¹⁰ is H.

In formula XIX, R⁹ is NR²²R²³, SH, NH₂, NH₃, or OH. R²² and R²³ are each independently H, methyl, ethyl, n-propyl, or isopropyl. In one or more embodiments, R⁹ is SH.

In one or more embodiments, R⁹ is OH.

In formulas XVI, XVII, and XIX, n3 is zero or an integer from 1 to 5. In one or more embodiments, n3 is 0. In one or more embodiments, n3 is 1. In one or more embodiments, n3 is 2. In one or more embodiments, n3 is 3. In one or more embodiments, n3 is 4. In one or more embodiments, n3 is 5.

In one or more embodiments the quinoline-containing monomer is

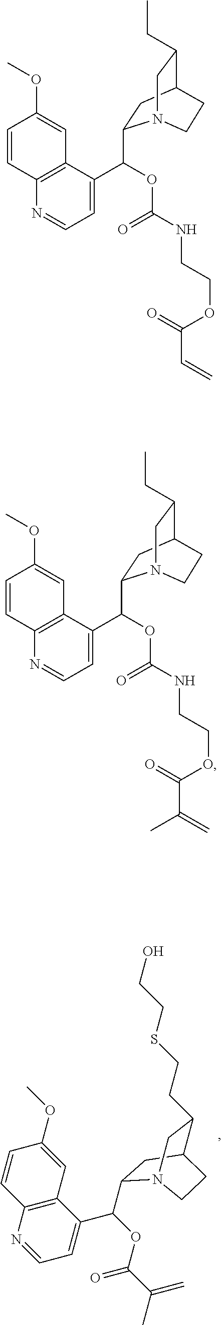 [SMILES: C=CC(=O)OCCNC(=O)OC(c1ccnc2ccc(OC)cc12)C1CC2CCN1CC2CC ; C=C(C)C(=O)OCCNC(=O)OC(c1ccnc2ccc(OC)cc12)C1CC2CCN1CC2CC ; C=C(C)C(=O)OC(c1ccnc2ccc(OC)cc12)C1CC2CCN1CC2CCSCCO]

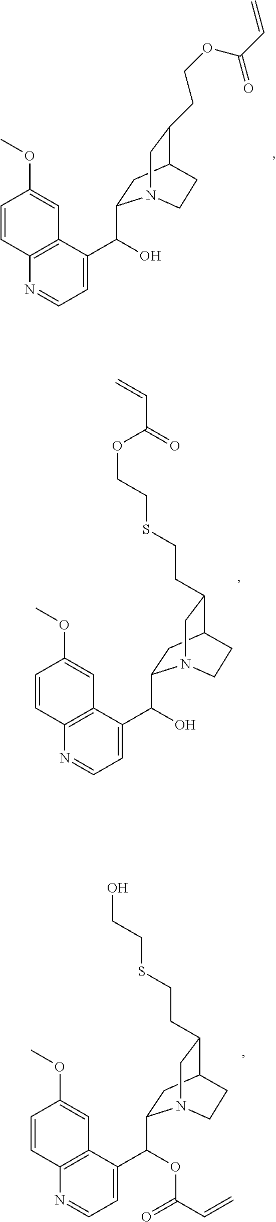 [SMILES: C=CC(=O)OCCC1CN2CCC1CC2C(O)c1ccnc2ccc(OC)cc12 ; C=CC(=O)OCCSCCC1CN2CCC1CC2C(O)c1ccnc2ccc(OC)cc12 ; C=CC(=O)OC(c1ccnc2ccc(OC)cc12)C1CC2CCN1CC2CCSCCO]

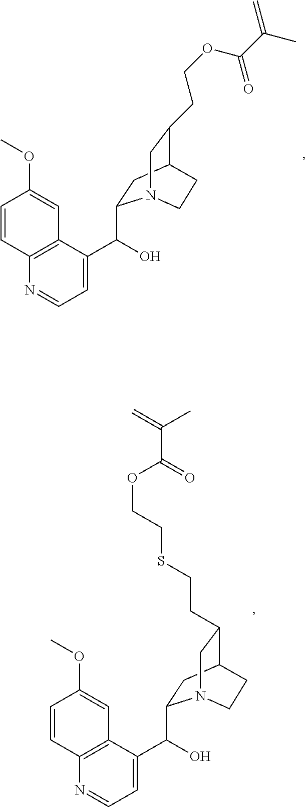 [SMILES: C=C(C)C(=O)OCCC1CN2CCC1CC2C(O)c1ccnc2ccc(OC)cc12 ; C=C(C)C(=O)OCCSCCC1CN2CCC1CC2C(O)c1ccnc2ccc(OC)cc12]

or combinations thereof.

In one or more embodiments, the quinoline-containing monomer is

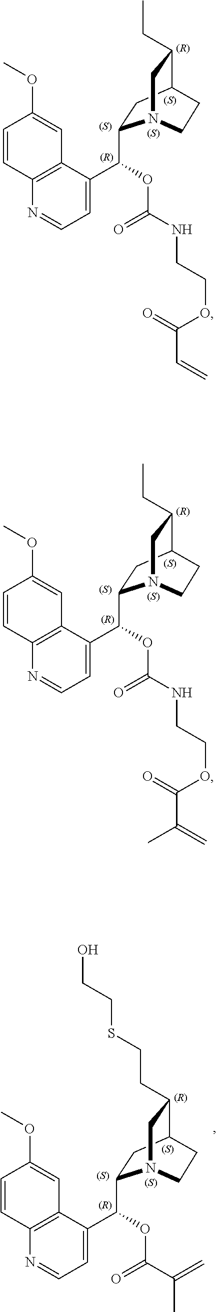

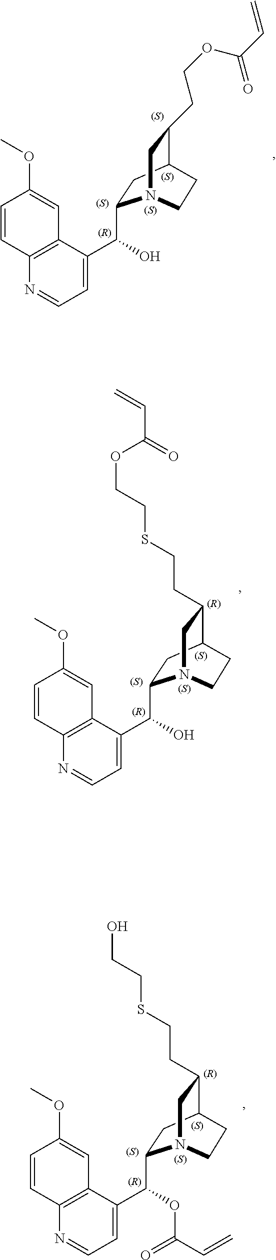

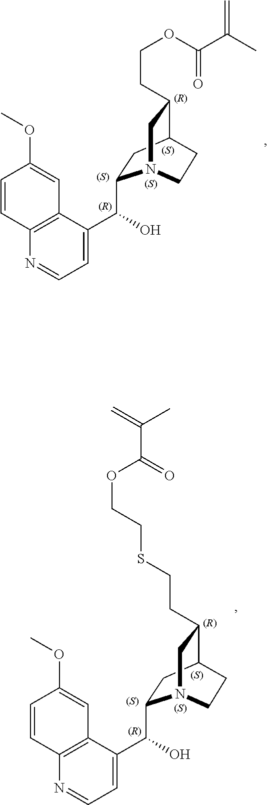

or combinations thereof.

In an aspect, the present disclosure describes polymers that include at least one of the quinoline-containing monomers of the present disclosure. Polymers are generally described by the monomers used to make them. As such, it is understood that when a polymer is described as including a particular monomer, the polymer includes the reaction product of the monomer with another monomer of the same kind or different kind. For example, when a monomer includes an alkene polymerizable group, upon polymerization the alkene becomes unsaturated. Specifically, polymerization of a —CHCH₂ polymerizable group results in a —CHCH₂— group where-represents a point of connection bond.

Polymers polymerized from a particular monomer may be described as “monomer name” polymer. For example, a polymer polymerized from a quinoline-containing monomer of the general formula IX, may be described as an IX polymer. Similarity, a copolymer polymerized from a quinoline-containing monomer of the general formula IX and an acrylamide monomer may be referred to as an IX-acrylamide polymer. The statement “polymerized from” is open ended and does not limit the polymer being described to being formed solely from the monomer described following the statement. Such polymer include other monomer. For example, a polymer polymerized from a quinoline-containing monomer may be formed from only the quinoline-containing monomer or may be formed from the quinoline-containing monomer and one or more additional monomers.

In one or more embodiments, the polymer may include more than one quinoline-containing monomer. For example, In one or more embodiments, the polymer may include one, two, three, four, or five quinoline-containing monomers.

In another aspect, the present disclosure describes a copolymer that includes a quinoline-containing monomer. A copolymer is polymer that includes at least two monomers. In one or more embodiments, the copolymer includes a first monomer and a second monomer. In some such embodiments, the first monomer is a quinoline-containing monomer and the second monomer is a monomer that is not a quinoline-containing monomer. The quinoline-containing monomer may be any quinoline-containing monomer described herein.

In one or more embodiments, the copolymer is an alternating copolymer. An alternating copolymer is a polymer where the first monomer and the second monomer alternate along the polymer chain.

In one or more embodiments, the copolymer is a statistical copolymer. A statistical copolymer is a polymer where the first monomer and the second monomer are arranged in a sequential order that follows a statistical rule. Examples of statistical rules that a copolymer may follow include, but are not limited to, a Bernoullian distribution (random copolymer) or Markovian distribution.

In one or more embodiments, the copolymer is a block copolymer. A block copolymer is a copolymer that includes two or more polymer segments (chains) joined by a covalent linkage. A block copolymer may include two or more homopolymer segments; two or more copolymer segments; or one or more homopolymer segments and one or more copolymer segments that are joined by a covalent linkage. For example, a block copolymer of the present disclosure may include a homopolymer of the first monomer and a homopolymer of the second monomer which are covalently joined to form a single polymer. A block copolymer of the present disclosure may include a first copolymer polymerized from two or more monomers covalently joined to a second copolymer polymerized from two or more monomers to form the block copolymer. A block copolymer of the present disclosure may include a first copolymer polymerized from two or more monomers covalently joined to homopolymer polymerized from a monomer to form a single polymer. The polymer segments making up the block copolymer may be polymerized from the same monomer or monomers, different monomers, or both. For example, a block copolymer may be formed by linking a first copolymer polymerized from a first monomer and a second monomer to a second copolymer polymerized from the first monomer and a third monomer.

In one or more embodiments, the block copolymers of the present disclosure may be triblock copolymers. A triblock copolymer is a block copolymer that includes three covalently linked polymer segments, a first terminal segment, a middle segment, and a second terminal segment. Each segment may be a homopolymer or a copolymer. For example, a triblock polymer may include two terminal homopolymer blocks of the first monomer with a central homopolymer block of the second monomer. In one or more embodiments, the triblock polymer includes two terminal homopolymer blocks of the second monomer with a central homopolymer block of the first monomer.

The amount of the quinoline-containing monomer in the copolymer may vary. The amount of the quinoline-containing monomer in the copolymer may affect the properties of the copolymer. The mole percent (mol-%) of the quinoline-containing monomer in the copolymer may be measured and calculated using ¹H NMR via methods known in the art. In one or more embodiments, the amount of the quinoline-containing monomer in the polymer is 1 mol-% or greater, 5 mol-% or greater, 10 mol-% or greater, 15 mol-% or greater, 20 mol-% or greater, 25 mol-% or greater, 30 mol-% or greater, 60 mol-% or greater, 80 mol-% or greater. In one or more embodiments, the amount of the quinoline-containing monomer is 100 mol-% or less, 80 mol-% or less, 60 mol-% or less, 30 mol-% or less, 25 mol-% or less, 20 mol-% or less, or 15 mol-% or less, 10 mol-% or less, or 5 mol-% or less. In one or more embodiments, the amount of the quinoline-containing monomer in the polymer is 1 mol-% to 100 mol-%, 1 mol-% to 80 mol-%, 1 mol-% to 60 mol-%, 1 mol-% to 30 mol-%, 1 mol-% to 25 mol-%, 1 mol-% to 20 mol-%, 1 mol-% to 15 mol-%, 1 mol-% to 10 mol-%, or 1 mol-% to 5 mol-%. In one or more embodiments, the amount of the quinoline-containing monomer in the polymer is 5 mol-% to 100 mol-%, 5 mol-% to 80 mol-%, 5 mol-% to 60 mol-%, 5 mol-% to 30 mol-%, 5 mol-% to 25 mol-%, 5 mol-% to 20 mol-%, 5 mol-% to 15 mol-%, or 5 mol-% to 10 mol-%. In one or more embodiments, the amount of the quinoline-containing monomer in the polymer is 10 mol-% to 100 mol-%, 10 mol-% to 80 mol-%, 10 mol-% to 60 mol-%, 10 mol-% to 30 mol-%, 10 mol-% to 25 mol-%, 10 mol-% to 20 mol-%, or 10 mol-% to 15 mol-%. In one or more embodiments, the amount of the quinoline-containing monomer in the polymer is 15 mol-% to 100 mol-%, 15 mol-% to 80 mol-%, 15 mol-% to 60 mol-%, 15 mol-% to 30 mol-%, 15 mol-% to 25 mol-%, or 15 mol-% to 20 mol-%. In one or more embodiments, the amount of the quinoline-containing monomer in the polymer is 20 mol-% to 100 mol-%, 20 mol-% to 80 mol-%, 20 mol-% to 60 mol-%, 20 mol-% to 30 mol-%, or 20 mol-% to 25 mol-%. In one or more embodiments, the amount of the quinoline-containing monomer in the polymer is 25 mol-% to 100 mol-%, 25 mol-% to 80 mol-%, 25 mol-% to 60 mol-%, or 25 mol-% to 30 mol-%. In one or more embodiments, the amount of the quinoline-containing monomer in the polymer is 30 mol-% to 100 mol-%, 30 mol-% to 80 mol-%, or 30 mol-% to 60 mol-%. In one or more embodiments, the amount of the quinoline-containing monomer in the polymer is 60 mol-% to 100 mol-% or 60 mol-% to 80 mol-%. In one or more embodiments, the amount of the quinoline-containing monomer in the polymer is 80 mol-% to 100 mol-%. In one or more embodiments, the amount of the quinoline-containing monomer in the polymer is 15 mol-% to 60 mol-%. In one or more embodiments, the amount of the quinoline-containing monomer in the polymer is 15 mol-% to 30 mol-%.

The second monomer in the copolymer may be any monomer suitable for polymerization. In one or more embodiments, the second monomer includes an alkene polymerizable functional group. In one or more embodiments, the monomer that includes an alkene polymerizable functional group is an acrylate, methacrylate, acrylamide, methacrylamide, or a vinyl monomer.

In one or more embodiments, the second monomer is an acrylate. Examples of acrylate monomers include, but are not limited to, benzyl acrylate; methyl acrylate; ethyl acrylate; n-propyl acrylate; isopropyl acrylate; iso-butyl acrylate; tert-butyl acrylate; sec-butyl acrylate; iso-decyl arylate; heptadecyl acrylate; ethyldiglycol acrylate; 4-hydroxybutyl acrylate; 2-hydroxyethyl acrylate (HEA); 2-ethylhexylacrylate; hydroxyethylcaprolactone acrylate; hydroxypropyl acrylate; lauryl acrylate; 2-propyleptyl acrylate; stearyl acrylate; poly(ethylene glycol) methyl ether acrylate; poly(ethylene glycol) acrylate; N-acryloylmorpholine; and benzhydryl acrylate. In one or more embodiments, the second monomer is HEA.

In one or more embodiments, the second monomer is a methacrylate. Examples of methacrylate monomers include, but are not limited to, benzyl methacrylate; methyl methacrylate; ethyl methacrylate; n-propyl methacrylate; isopropyl methacrylate; iso-butyl methacrylate; tert-butyl methacrylate; sec butyl methacrylate; iso-decyl methacrylate; heptadecyl methacrylate; ethyldiglycol methacrylate; 4-hydroxybutyl methacrylate; 2-hydroxyethyl methacrylate (HEMA); 2-ethylhexyl methacrylate; hydroxyethylcaprolactone methacrylate; hydroxypropyl methacrylate; lauryl methacrylate; 2-propyleptyl methacrylate; stearyl methacrylate; N-methacryloylmorpholine; benzhydryl methacrylate; poly(ethylene glycol) methyl ether methacrylate; poly(ethylene glycol) methacrylate; and 2-n-Butoxyethyl methacrylate. In one or more embodiments, the second monomer is methyl methacrylate.

In one or more embodiments, the second monomer is an acrylamide. Examples of acrylamide monomers include, but are not limited to, acrylamide; N,N-dimethylacrylamide (DMA), N-isopropylacrylamide, N-ethylmethylacrylamide; N-ethyl acrylamide; poly(ethylene glycol) methyl ether acrylamide; poly(ethylene glycol) acrylamide; and (2-hydroxyethyl) acrylamide. In one or more embodiments, the second monomer is (2-hydroxyethyl) acrylamide. In one or more embodiments, the second monomer is N-isopropyl acrylamide. In one or more embodiments, the second monomer is N,N-dimethylacrylamide.

In one or more embodiments, the second monomer is a methacrylamide. Examples of methacrylamide monomers include, but are not limited to, methacrylamide; N,N-dimethylmethacrylamide; N-isopropylmethacrylamide; N-isopropyl methylmethacrylamide; N,N-dimethylmethacrylamide; N-ethylmethylmethacrylamide; N-ethyl methacrylamide; poly(ethylene glycol) methyl ether methacrylamide; poly(ethylene glycol) methacrylamide; and (2-hydroxyethyl) methacrylamide. In one or more embodiments, the second monomer is (2-hydroxyethyl) methacrylamide. In one or more embodiments, the second monomer is N-isopropyl methacrylamide. In one or more embodiments, the second monomer is N,N-dimethylmethacrylamide.

In one or more embodiments, the second monomer is a vinyl monomer. In one or more embodiments, the vinyl monomer is a vinyl ester, vinyl amide, or a vinyl ether. Examples of vinyl monomers include, but are not limited to, vinyl acetate; vinyl cinnamate; ethyl vinyl ether; acrylonitrile; ethylene; propylene; styrene; vinyl chloride; vinyl propionate; vinyl laurate; ethyl vinyl ether; iso-butyl vinyl ether; cyclohexyl vinyl ether; dodecyl vinyl ether; octadecyl vinyl ether; hydroxyl butyl vinyl ether; 3-amin propyl vinyl ether; N-vinyl-N-methyl acetamide; N-vinyl imidazole; N-vinyl pyrrolidone; and vinyl methyl oxazolidinone. In one or more embodiments, the second monomer is vinyl acetate.

The number-average molecular weight (Mn) of the polymers and/or the copolymers of the present application may vary. Mn is calculated using the following equation:

$$Mn = \sum {x}_{i} {M}_{i}$$

where Mᵢ is the mean molecular size of range i and xᵢ is the number fraction of the total number of polymer chains that are within Mi range. Mn may be determined using size exclusion chromatography with a multi-angle light scattering detector (see Example).

In one or more embodiments, the Mn of the polymer is 5 kilodalton (kDa) or greater, 10 kDa or greater, 20 kDa or greater, 25 kDa or greater, 30 kDa or greater, 40 kDa or greater, 50 kDa or greater, or 75 kDa or greater. In one or more embodiments, the Mn of the polymer is 100 kDa or less, 75 kDa or less, 50 kDa or less, 40 kDa or less, 30 kDa or less, 25 kDa or less, 20 kDa or less, or 10 kDa or less. In one or more embodiments, the Mn of the polymer is 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the Mn of the polymer is 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mn of the polymer is 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mn of the polymer is 25 kDa to 100 kDa, 25 kDa to 75 kDa, 25 kDa to 50 kDa, 25 kDa to 40 kDa, or 25 kDa to 30 kDa. In one or more embodiments, the Mn of the polymer is 30 kDa to 100 kDa, 30 kDa to 75 kDa, 30 kDa to 50 kDa, or 30 kDa to 40 kDa. In one or more embodiments, the Mn of the polymer is 40 kDa to 100 kDa, 40 kDa to 75 kDa, or 40 kDa to 50 kDa. In one or more embodiments, the Mn of the polymer is 50 kDa to 100 kDa or 50 kDa to 75 kDa. In one or more embodiments, the Mn of the polymer is 75 kDa to 100 kDa.

The weight-average molecular weight (Mw) of the polymers of the present application may vary. Mw is calculated using the following equation:

$$Mw = \sum {w}_{i} {M}_{i}$$

where Mᵢ is the mean molecular size of range i and wᵢ is the weight fraction of the total number of polymer chains that are within Mᵢ range. Mw may be determined using size exclusion chromatography with a multi-angle light scattering detector (see Example).

In one or more embodiments, the Mw of the polymer is 5 kilodalton (kDa) or greater, 10 kDa or greater, 20 kDa or greater, 25 kDa or greater, 30 kDa or greater, 40 kDa or greater, 50 kDa or greater, or 75 kDa or greater. In one or more embodiments, the Mw of the polymer is 100 kDa or less, 75 kDa or less, 50 kDa or less, 40 kDa or less, 30 kDa or less, 25 kDa or less, 20 kDa or less, or 10 kDa or less. In one or more embodiments, the Mw of the polymer is 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the Mw of the polymer is 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mw of the polymer is 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mw of the polymer is 25 kDa to 100 kDa, 25 kDa to 75 kDa, 25 kDa to 50 kDa, 25 kDa to 40 kDa, or 25 kDa to 30 kDa. In one or more embodiments, the Mw of the polymer is 30 kDa to 100 kDa, 30 kDa to 75 kDa, 30 kDa to 50 kDa, or 30 kDa to 40 kDa. In one or more embodiments, the Mw of the polymer is 40 kDa to 100 kDa, 40 kDa to 75 kDa, or 40 kDa to 50 kDa. In one or more embodiments, the Mw of the polymer is 50 kDa to 100 kDa or 50 kDa to 75 kDa. In one or more embodiments, the Mw of the polymer is 75 kDa to 100 kDa.

The dispersity of the molecular weight of the polymer affect the characteristics of the polymer. The molecular weight dispersity may be quantified as the dispersity (Ð_{M}). Ð_{M} is the distribution of individual molecular masses of a polymer. Ð_{M} is calculated as the quotient of the mass average molecular weight (M_{w}) divided by the number-average molecular weight (Mₙ). The M_{w} and Mₙ may be determined using various methods including viscometry, size exclusion chromatography, and mass spectrometry. Generally, a small Ð_{M} is preferred. Although there is no desired lower limit, in practice the Ð_{M} of the polymer may be 1.0 or greater, 1.1 or greater, 1.2 or greater, 1.3 or greater, 1.4 or greater, 1.5 or greater, 1.6 or greater, 1.7 or greater, 1.8 or greater, 1.9 or greater, 2.0 or greater, 2.1 or greater, 2.2 or greater, 2.3 or greater. 2.3 or greater, or 2.4 or greater. In some embodiments, the Ð_{M} of the polymer may be 2.5 or less, 2.2 or less, 2.0 or less, 1.8 or less, 1.7 or less, 1.6 or less, 1.5 or less, 1.4 or less, 1.3 or less, 1.2 or less, or 1.1 or less. In some embodiments, the Ð_{M} for the polymer is 1.0 to 2.5, 1.0 to 2.2, 1.0 to 2.0, 1.0 to 1.8, 1.0 to 1.7, 1.0 to 1.6, 1.0 to 1.5, 1.0 to 1.4, 1.0 to 1.3, 1.0 to 1.2, or 1.0 to 1.1. In some embodiments, the Ð_{M} for the polymer is 1.1 to 2.5, 1.1 to 2.2, 1.1 to 2.0, 1.1 to 1.8, 1.1 to 1.7, 1.1 to 1.6, 1.1 to 1.5, 1.1 to 1.4, 1.1 to 1.3, or 1.1 to 1.2. In some embodiments, the Ð_{M} for the polymer is 1.2 to 2.5, 1.2 to 2.2, 1.2 to 2.0, 1.2 to 1.8, 1.2 to 1.7, 1.2 to 1.6, 1.2 to 1.5, 1.2 to 1.4, or 1.2 to 1.3. In some embodiments, the Ð_{M} for the polymer is 1.3 to 2.5, 1.3 to 2.2, 1.3 to 2.0, 1.3 to 1.8, 1.3 to 1.7, 1.3 to 1.6, 1.3 to 1.5, or 1.3 to 1.4. In some embodiments, the Ð_{M} for the polymer is 1.4 to 2.5, 1.4 to 2.2, 1.4 to 2.0, 1.4 to 1.8, 1.4 to 1.7, 1.4 to 1.6, or 1.4 to 1.5. In some embodiments, the Ð_{M} for the polymer is 1.5 to 2.5, 1.5 to 2.2, 1.5 to 2.0, 1.5 to 1.8, 1.5 to 1.7, or 1.5 to 1.6. In some embodiments, the Ð_{M} for the polymer is 1.6 to 2.5, 1.6 to 2.2, 1.6 to 2.0, 1.6 to 1.8, or 1.6 to 1.7. In some embodiments, the Ð_{M} for the polymer is 1.7 to 2.5, 1.7 to 2.2, 1.7 to 2.0, or 1.7 to 1.8. In some embodiments, the Ð_{M} for the polymer is 1.8 to 2.5, 1.8 to 2.2, or 1.8 to 2.0. In some embodiments, the Ð_{M} for the polymer is 2.0 to 2.5, 2.0 to 2.2, or 2.2 to 2.5.

The pKa of a polymer affect the characteristics of the polymer. For example, the pKa of a polymer may affect the ability of the polymer to form complexes (e.g., polyplexes) with oligonucleotides and/or the ability to release oligonucleotides from a polymer-oligonucleotide complex. The pKa of a polymer may be determined using a potentiometric titration (see Example). In one or more embodiments, the pKa of the polymer is 5.0 or greater, 5.5 or greater, 6.0 or greater, 6.1 or greater, 6.2 or greater, 6.3 or greater, 6.4 or greater, 6.5 or greater, 6.6 or greater, 6.7 or greater, 6.8 or greater, 6.9 or greater, 7.0 or greater, 7.2 or greater, or 7.5 or greater. In one or more embodiments, the pKa of the polymer is 8.0 or less, 7.5 or less, 7.2 or less, 7.0 or less, 6.9 or less, 6.8 or less, 6.7 or less, 6.6 or less, 6.5 or less, 6.4 or less, 6.3 or less, 6.2 or less, 6.1 or less, 6.0 or less, or 5.5 or less. In one or more embodiments the pKa of the polymer is 5.0 to 8.0, 5.0 to 7.5, 5.0 to 7.2, 5.0 to 7.0, 5.0 to 6.9, 5.0 to 6.8, 5.0 to 6.7, 5.0 to 6.6, 5.0 to 6.5, 5.0 to 6.4, 5.0 to 6.3, 5.0 to 6.2, 5.0 to 6.1, 5.0 to 6.0, or 5.0 to 5.5. In one or more embodiments the pKa of the polymer is 5.5 to 8.0, 5.5 to 7.5, 5.5 to 7.2, 5.5 to 7.0, 5.5 to 6.9, 5.5 to 6.8, 5.5 to 6.7, 5.5 to 6.6, 5.5 to 6.5, 5.5 to 6.4, 5.5 to 6.3, 5.5 to 6.2, 5.5 to 6.1, or 5.5 to 6.0. In one or more embodiments the pKa of the polymer is 6.0 to 8.0, 6.0 to 7.5, 6.0 to 7.2, 6.0 to 7.0, 6.0 to 6.9, 6.0 to 6.8, 6.0 to 6.7, 6.0 to 6.6, 6.0 to 6.5, 6.0 to 6.4, 6.0 to 6.3, 6.0 to 6.2, or 6.0 to 6.1. In one or more embodiments the pKa of the polymer is 6.1 to 8.0, 6.1 to 7.5, 6.1 to 7.2, 6.1 to 7.0, 6.1 to 6.9, 6.1 to 6.8, 6.1 to 6.7, 6.1 to 6.6, 6.1 to 6.5, 6.1 to 6.4, 6.1 to 6.3, or 6.1 to 6.2. In one or more embodiments the pKa of the polymer is 6.2 to 8.0, 6.2 to 7.5, 6.2 to 7.2, 6.2 to 7.0, 6.2 to 6.9, 6.2 to 6.8, 6.2 to 6.7, 6.2 to 6.6, 6.2 to 6.5, 6.2 to 6.4, or 6.2 to 6.3. In one or more embodiments the pKa of the polymer is 6.3 to 8.0, 6.3 to 7.5, 6.3 to 7.2, 6.3 to 7.0, 6.3 to 6.9, 6.3 to 6.8, 6.3 to 6.7, 6.3 to 6.6, 6.3 to 6.5, or 6.3 to 6.4. In one or more embodiments the pKa of the polymer is 6.4 to 8.0, 6.4 to 7.5, 6.4 to 7.2, 6.4 to 7.0, 6.4 to 6.9, 6.4 to 6.8, 6.4 to 6.7, 6.4 to 6.6, or 6.4 to 6.5. In one or more embodiments the pKa of the polymer is 6.5 to 8.0, 6.5 to 7.5, 6.5 to 7.2, 6.5 to 7.0, 6.5 to 6.9, 6.5 to 6.8, 6.5 to 6.7, or 6.5 to 6.6. In one or more embodiments the pKa of the polymer is 6.6 to 8.0, 6.6 to 7.5, 6.6 to 7.2, 6.6 to 7.0, 6.6 to 6.9, 6.6 to 6.8, or 6.6 to 6.7. In one or more embodiments the pKa of the polymer is 6.7 to 8.0, 6.7 to 7.5, 6.7 to 7.2, 6.7 to 7.0, 6.7 to 6.9, or 6.7 to 6.8. In one or more embodiments the pKa of the polymer is 6.8 to 8.0, 6.8 to 7.5, 6.8 to 7.2, 6.8 to 7.0, or 6.8 to 6.9. In one or more embodiments the pKa of the polymer is 6.9 to 8.0, 6.9 to 7.5, 6.9 to 7.2, or 6.9 to 7.0. In one or more embodiments the pKa of the polymer is 7.0 to 8.0, 7.0 to 7.5, or 7.0 to 7.2. In one or more embodiments the pKa of the polymer is 7.2 to 8.0, 7.2 to 7.5, or 7.5 to 8.0.

The polymers of the present disclosure include protonatable nitrogens. For example, when quinine monomers are included in the polymer the quine has two nitrogens that can be protonated one at the quinuclidine group (pKₐ=8.5) and the other at the quinoline ring (pKₐ=4.1). The protonation of one of a first nitrogen in a polymer can affect the protonation of second nitrogen in a polymer. The Hill coefficient (n_{Hill}) is a measure of cooperativity in the protonation-deprotonation process. A value of n_{Hill}~ 1 implies that the amine groups in the polymer undergo protonation (or deprotonation) independent of other amine groups in their proximity. Whereas n_{Hill}>1 suggests positive cooperativity in protonation (or deprotonation), which means protonation (or deprotonation) of one amine group facilitates protonation (or deprotonation) of the surrounding amine groups.

The polymers of the present disclosure may have a variety of n_{Hill} values. In one or more embodiments, the polymer has an n_{Hill} of 0.5 or greater, 0.7 or greater, 1.0 or greater, 1.1 or greater, 1.2 or greater, 1.3 or greater, 1.4 or greater, 1.5 or greater, 1.6 or greater, 1.7 or greater, 1.8 or greater, 1.9 or greater, 2.0 or greater, or 2.2 or greater. In one or more embodiments, the polymer has a has an n_{Hill} of 2.5 or less, 2.2 or less, 2.0 or less, 1.9 or less, 1.8 or less, 1.7 or less, 1.6 or less, 1.5 or less, 1.4 or less, 1.3 or less, 1.2 or less, 1.1 or less, 1.0 or less, or 0.7 or less. In one or more embodiments, the polymer has an n_{Hill} of 0.5 to 2.5, 0.5 to 2.2, 0.5 to 2.0, 0.5 to 1.9, 0.5 to 1.8, 0.5 to 1.7, 0.5 to 1.6, 0.5 to 1.5, 0.5 to 1.4, 0.5 to 1.3, 0.5 to 1.2, 0.5 to 1.1, 0.5 to 1.0, or 0.5 to 0.7. In one or more embodiments, the polymer has an n_{Hill} of 0.7 to 2.5, 0.7 to 2.2, 0.7 to 2.0, 0.7 to 1.9, 0.7 to 1.8, 0.7 to 1.7, 0.7 to 1.6, 0.7 to 1.5, 0.7 to 1.4, 0.7 to 1.3, 0.7 to 1.2, 0.7 to 1.1, or 0.7 to 1.0. In one or more embodiments, the polymer has an n_{Hill} of 1.0 to 2.5, 1.0 to 2.2, 1.0 to 2.0, 1.0 to 1.9, 1.0 to 1.8, 1.0 to 1.7, 1.0 to 1.6, 1.0 to 1.5, 1.0 to 1.4, 1.0 to 1.3, 1.0 to 1.2, or 1.0 to 1.1. In one or more embodiments, the polymer has an n_{Hill} of 1.1 to 2.5, 1.1 to 2.2, 1.1 to 2.0, 1.1 to 1.9, 1.1 to 1.8, 1.1 to 1.7, 1.1 to 1.6, 1.1 to 1.5, 1.1 to 1.4, 1.1 to 1.3, or 1.1 to 1.2. In one or more embodiments, the polymer has an n_{Hill} of 1.2 to 2.5, 1.2 to 2.2, 1.2 to 2.0, 1.2 to 1.9, 1.2 to 1.8, 1.2 to 1.7, 1.2 to 1.6, 1.2 to 1.5, 1.2 to 1.4, or 1.2 to 1.3. In one or more embodiments, the polymer has an num of 1.3 to 2.5, 1.3 to 2.2, 1.3 to 2.0, 1.3 to 1.9, 1.3 to 1.8, 1.3 to 1.7, 1.3 to 1.6, 1.3 to 1.5, or 1.3 to 1.4. In one or more embodiments, the polymer has an n_{Hill} of 1.4 to 2.5, 1.4 to 2.2, 1.4 to 2.0, 1.4 to 1.9, 1.4 to 1.8, 1.4 to 1.7, 1.4 to 1.6, or 1.4 to 1.5. In one or more embodiments, the polymer has an n_{Hill} of 1.5 to 2.5, 1.5 to 2.2, 1.5 to 2.0, 1.5 to 1.9, 1.5 to 1.8, 1.5 to 1.7, or 1.5 to 1.6. In one or more embodiments, the polymer has an n_{Hill} of 1.6 to 2.5, 1.6 to 2.2, 1.6 to 2.0, 1.6 to 1.9, 1.6 to 1.8, or 1.6 to 1.7. In one or more embodiments, the polymer has an n_{Hill} of 1.7 to 2.5, 1.7 to 2.2, 1.7 to 2.0, 1.7 to 1.9, or 1.7 to 1.8. In one or more embodiments, the polymer has an n_{Hill} of 1.8 to 2.5, 1.8 to 2.2, 1.8 to 2.0, or 1.8 to 1.9. In one or more embodiments, the polymer has an n_{Hill} of 1.9 to 2.5, 1.9 to 2.2, or 1.9 to 2.0. In one or more embodiments, the polymer has an n_{Hill} of 2.0 to 2.5, 2.0 to 2.2, or 2.2 to 2.5.

In one or more embodiments, the copolymer may include additional monomers beyond the first and second monomers. In one or more embodiments, the copolymer may include a third, fourth, or fifth monomer. Any additional monomers may be any of the quinoline-containing monomers as described elsewhere herein. Any additional monomers may be acrylates, methacrylates, acrylamides, methacrylamides, vinyl monomers, quinoline-containing monomers, or combinations thereof.

In another aspect, the present disclosure describes a method of forming a polymer that includes a quinoline-containing monomer. The quinoline-containing monomer may be any quinoline-containing monomer as described elsewhere herein. In one or more embodiments, the polymer including the quinoline-containing monomer is a homopolymer. In one or more embodiments, the polymer including the quinoline-containing monomer is a copolymer. In such embodiments, the copolymer includes a first monomer and a second monomer. The first monomer is the quinoline-containing monomer and the second monomer may be any second monomer as described herein.

In one or more embodiments, forming the polymer includes making a reaction mixture. The reaction mixture may include the quinoline-containing monomer, the optional second monomer, a solvent, and an initiator. Any suitable initiator may be used. Examples of initiators include, but are not limited to, are azobisisobutyronitrile (AIBN), benzoyl peroxide, and 4,4′-azobis(4-cyanovaleric acid) (ACVA). Generally, initiators used have a half-life of 10 hours or greater at the temperature of the reaction mixture. Any suitable solvent may be used. Examples of solvents include, but are not limited to, dimethylformamide, dimethyl sulfoxide, tetrahydrofuran, dioxane, dichloromethane, chloroform, ethyl acetate, and combinations thereof.

In one or more embodiments, forming the polymer includes allowing reverse addition-fragmentation chain transfer (RAFT) polymerization to occur. In such embodiments, the reaction mixture may include the quinoline-containing monomer, the optional second monomer, the solvent, the initiator, and a chain transfer agent (sometimes called a RAFT agent). Any suitable chain transfer reagent may be used. Examples of chain transfer agents include, but are not limited to, dithiobenzoates, trithiocarbonates, xanthates, and dithiocarbamates. The choice of a specific chain transfer agents within each class of chain transfer agents is based on the solubility and/or compatibility with other components of the reaction. In some cases, the choice of a specific chain transfer agents within each class of chain transfer agents is based on availability of the chain transfer agent. In one or more embodiments, custom chain transfer agents may be synthesized, for example, to include a targeting group.

In one or more embodiments, forming the polymer includes allowing free radical polymerization (FRP) to occur. In such embodiments, the reaction mixture may include the quinoline-containing monomer, the optional second monomer, the solvent, and the initiator.

In one or more embodiments, forming the polymer is done under a nitrogen atmosphere.

In one or more embodiments, forming the polymer is done at elevated temperature.

In one or more embodiments, the method includes forming a pre-reaction mixture. The pre-reaction mixture may include the quinoline-containing monomer and the solvent. In one or more embodiments, the pre-reaction mixture may also include the optional second monomer and/or a chain transfer agent. In one or more embodiments, the pre-reaction mixture is heated. Heating the pre-reaction mixture may allow the quinoline-containing monomer to dissolve and/or disperse throughout the solvent to create a homogenous pre-reaction mixture.

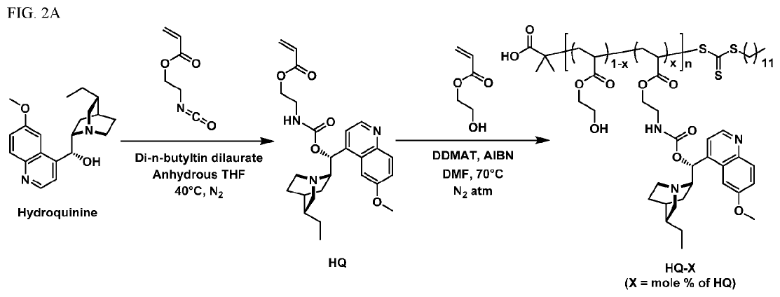

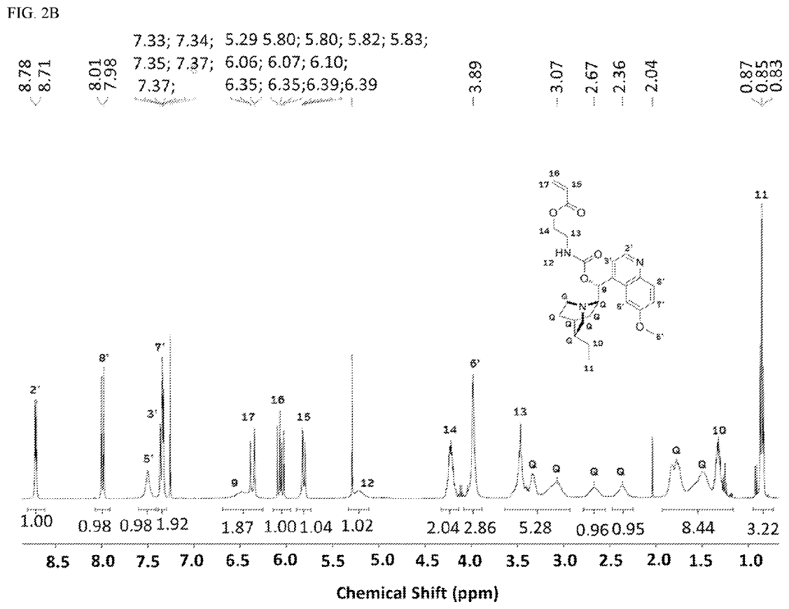

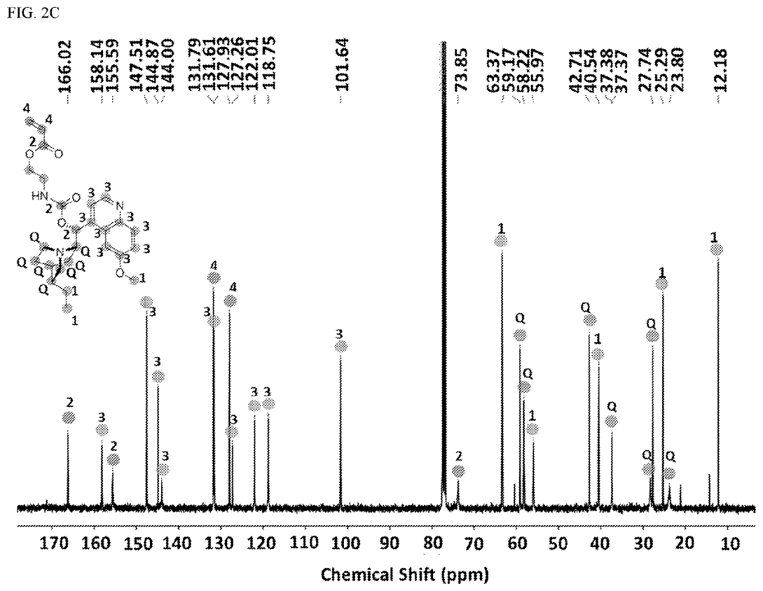

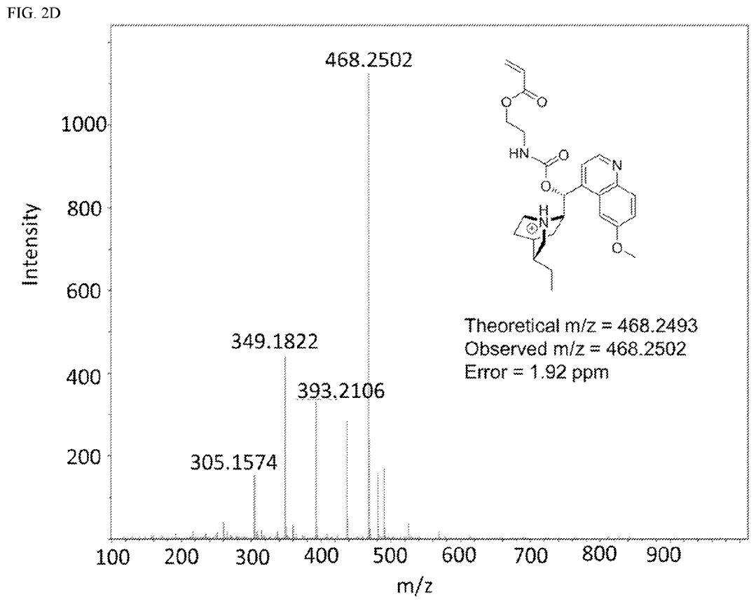

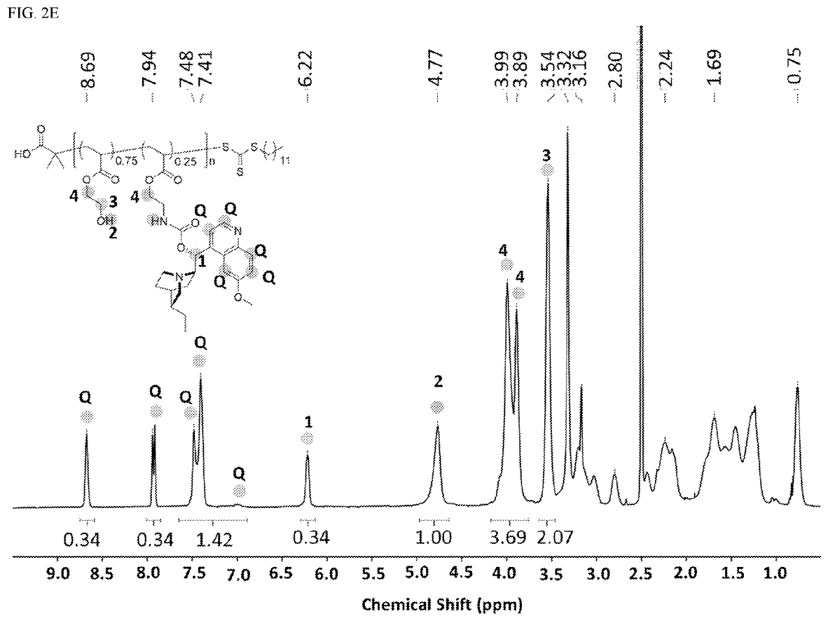

Several quinoline containing polymers were synthesized and characterized. A quinoline containing monomer that is an acrylate analog of hydroquinine (HQ) was synthesized by coupling hydroquinine with 2-isocyanatoethyl acrylate (FIG. 2A) and characterize via ¹H NMR (FIG. 2B), ¹³C NMR (FIG. 2C) and high resolution mass spectrometry (FIG. 2D). The single polymerizable alkene on the final monomer of HQ may allow minimization of undesired chain transfer or crosslinking events during polymerization.

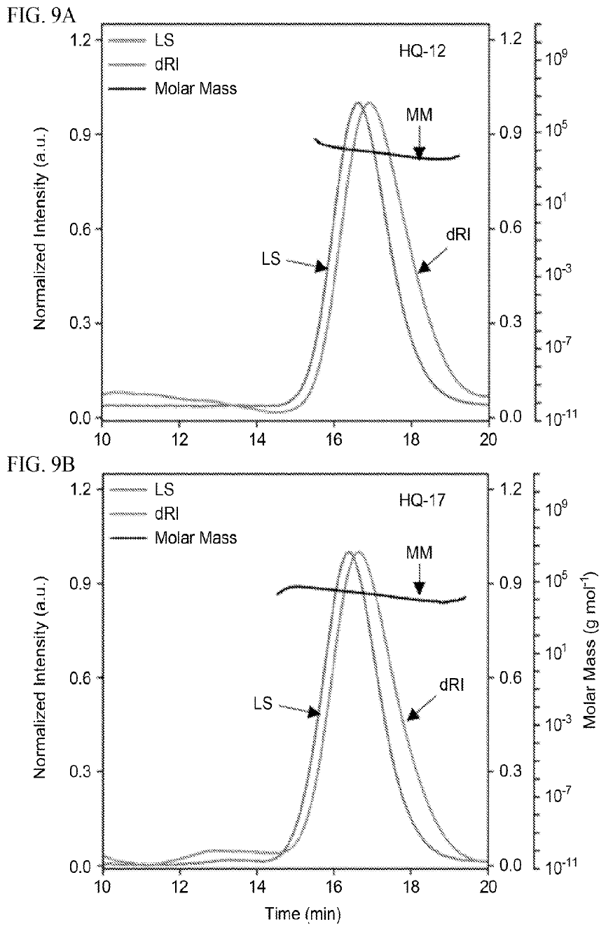

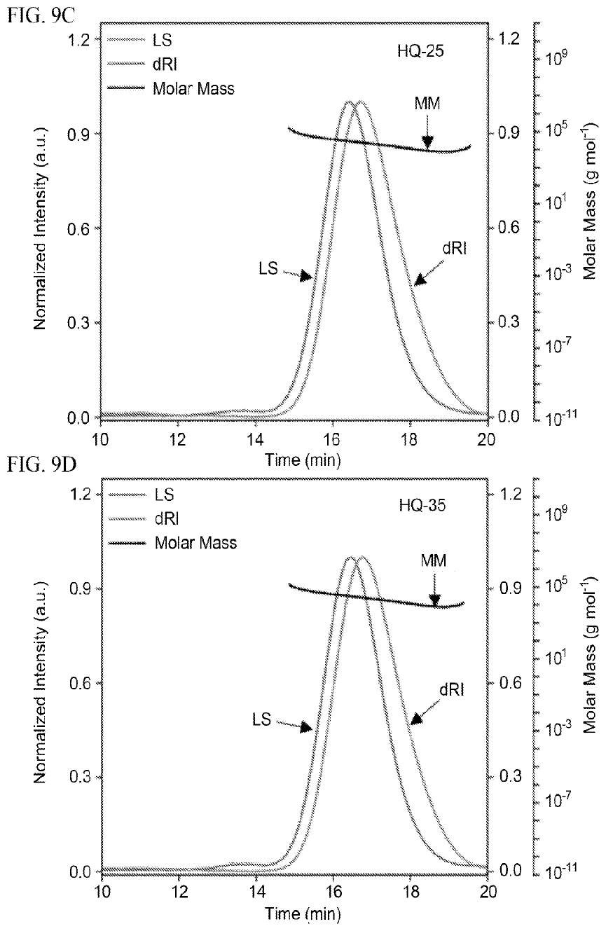

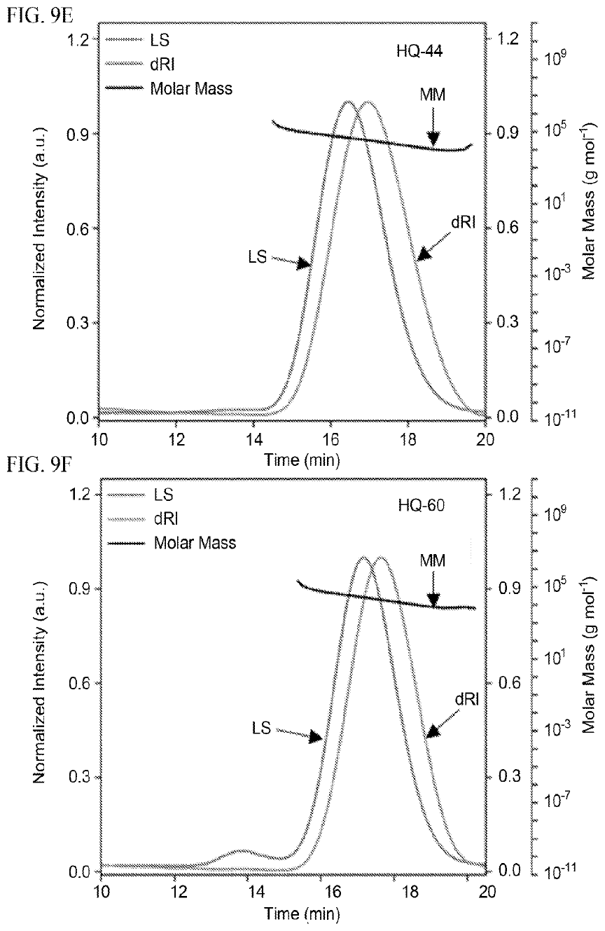

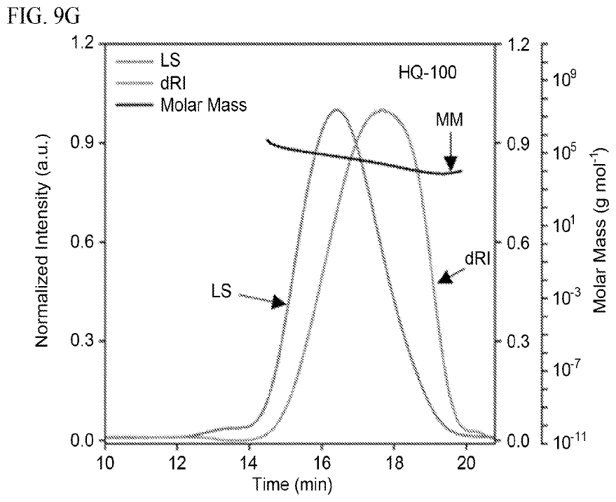

HQ was found to be compatible with RAFT copolymerization. A series of copolymers of HQ and HEA, with mole percentage of HQ in the polymer chain varying from 12% to 60%, were synthesized via RAFT copolymerization (FIG. 2A, Table 1A, FIGS. 9A-9G) and characterized using ¹H NMR (FIG. 2E). A homopolymer of HQ was also synthesized using free radical polymerization. This family of polymers consisted of the six copolymers synthesized via RAFT and the homopolymer of HQ synthesized via free radical polymerization, and are collectively referred to as HQ-X polymers, where X represents the mole percentage of HQ in the polymer. Table 1A shows the properties of the HQ-X polymers including Mw, Mn, dispersity, pKa, and n_{Hill} (see Example for how the properties were determined). Table 1B shows the feed compositions used in polymerization reactions, percentage conversion of the monomers, and the dn/dc value of the purified polymers. In Table 1B, an * indicates that the conversion couldn't be accurately estimated due to overlaps between the peaks in the ¹H NMR of the reaction mixture

TABLE 1A

| | | % HQ | M_{w} | Mₙ | | | |
|---|---|---|---|---|---|---|---|
| Code | Method | in chain | (kDa) | (kDa) | Ð | pKₐ | n_{Hill} |
| HQ-12 | RAFT | 12 | 19 | 16 | 1.24 | 7 | 0.9 |
| HQ-17 | RAFT | 17 | 22 | 17 | 1.29 | 6.6 | 1.3 |
| HQ-25 | RAFT | 25 | 24 | 19 | 1.28 | 6.6 | 1.5 |
| HQ-35 | RAFT | 35 | 25 | 19 | 1.28 | 6.3 | 1.7 |
| HQ-44 | RAFT | 44 | 35 | 25 | 1.39 | 6.3 | 1.7 |
| HQ-60 | RAFT | 60 | 21 | 16 | 1.33 | 6.2 | 1.7 |
| HQ-100 | FRP | 100 | 39 | 20 | 1.91 | 6.2 | 1.6 |

TABLE 1B

| | % HQ | Conversion (%) | | | dn/dc |
|---|---|---|---|---|---|
| Code | (Feed) | HEA | HQ | Total | (mL/g) |
| HQ-12 | 10 | 70 | 57 | 68 | 0.0794 |
| HQ-17 | 15 | 75 | 68 | 74 | 0.0902 |
| HQ-25 | 25 | 75 | 73 | 75 | 0.1067 |
| HQ-35 | 35 | 68 | 71 | 69 | 0.1232 |
| HQ-44 | 50 | 60 | 60 | 60 | 0.1031 |
| HQ-60 | 75 | * | 35 | * | 0.1264 |
| HQ-100 | 100 | — | 90 | 90 | 0.1249 |

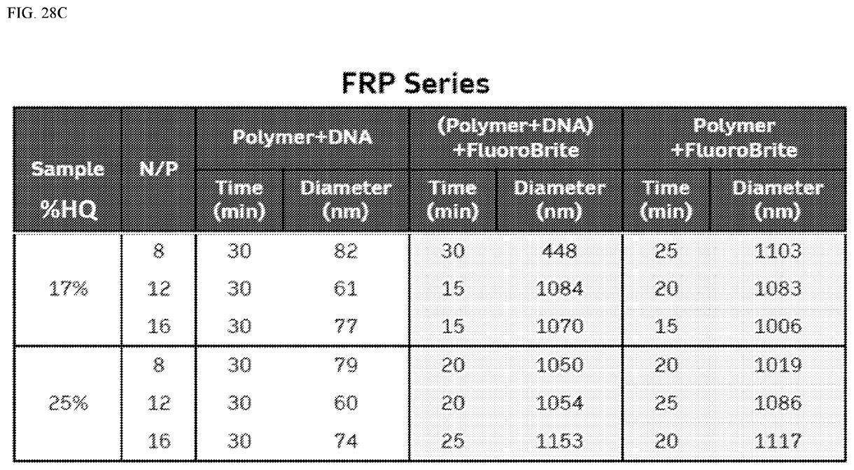

A second series copolymers having HQ and HEA where synthesized using free radical polymerization (FRP; FIG. 28A) and characterized for percent conversion of HEA and HQ; mol-% HQ in the polymer; Mw, Mn, Ð, and dn/dc (FIG. 28B). The polymers made in this series are referred to as FRP HQ-X polymers where X refers to the mol-% of HQ in the polymer. The dispersity, Mn, and Mw of the polymers made using FRP was larger than for the polymers made via RAFT (FIG. 28B and Table 1A).

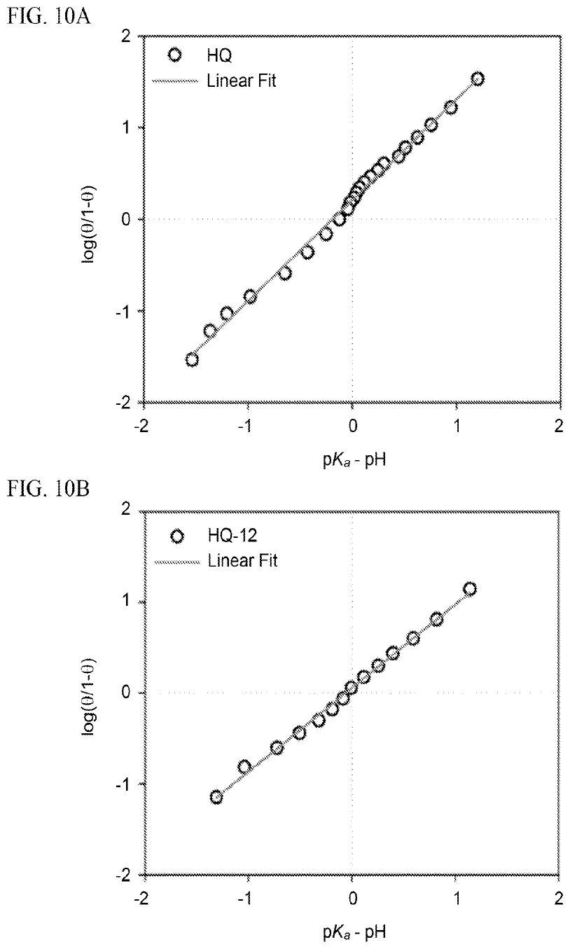

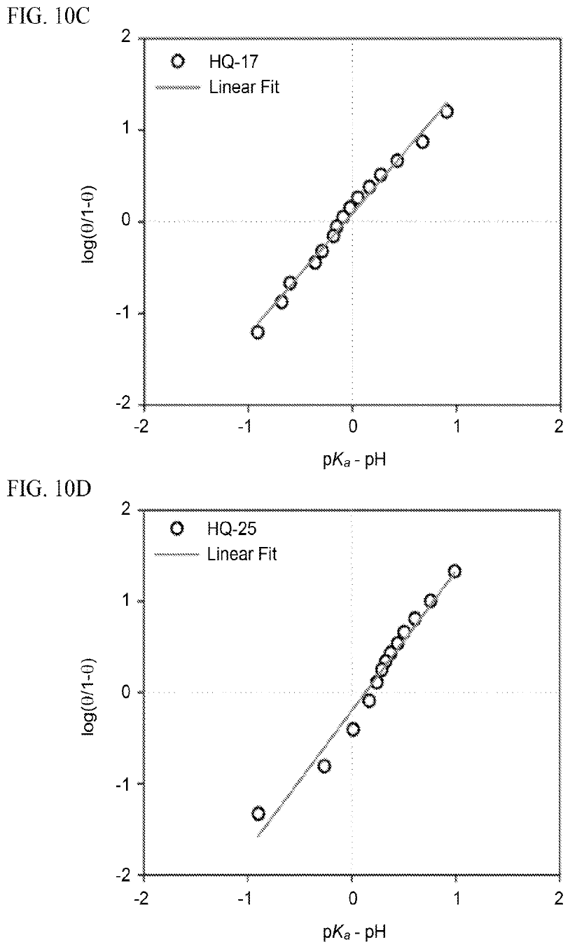

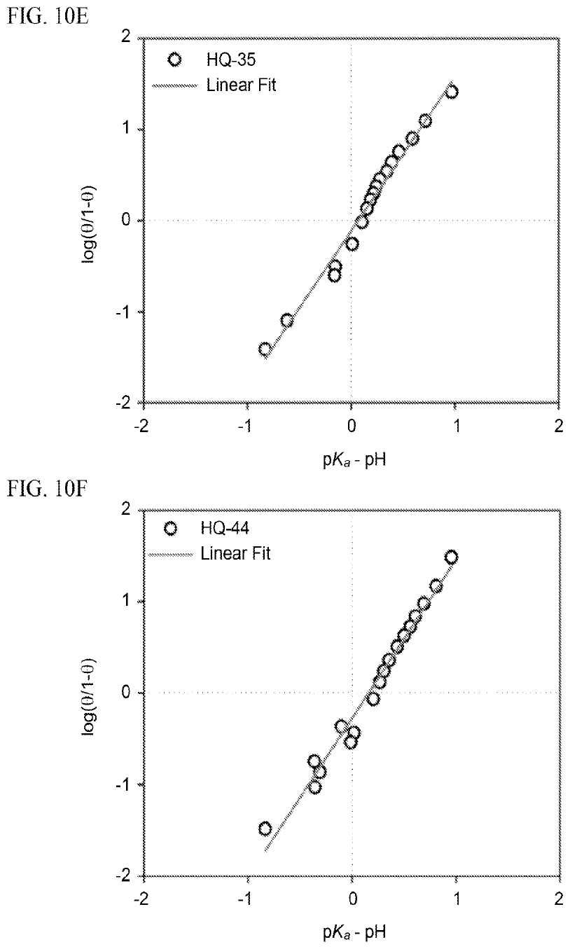

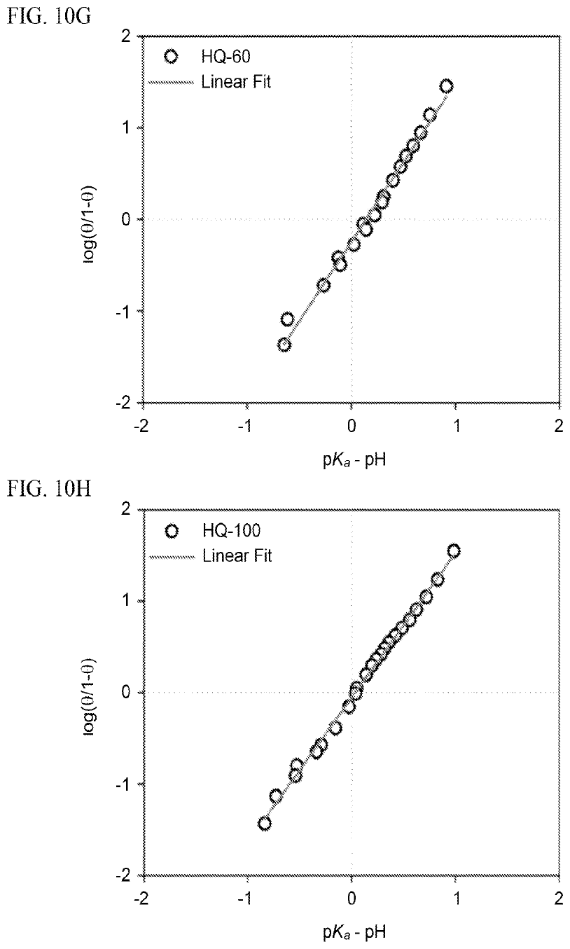

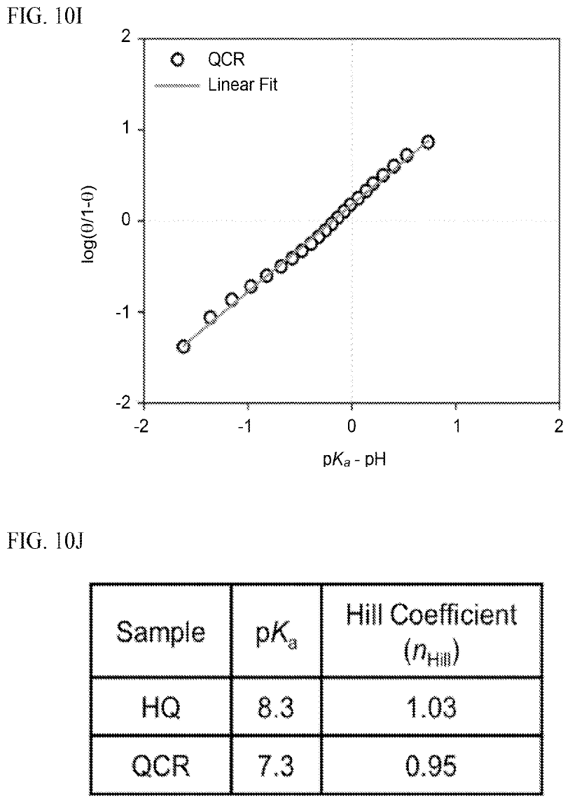

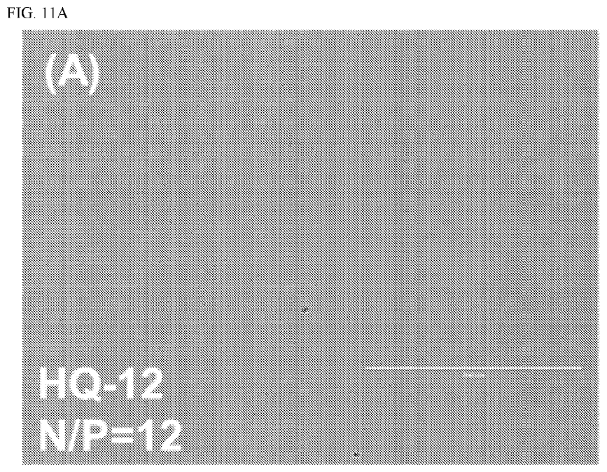

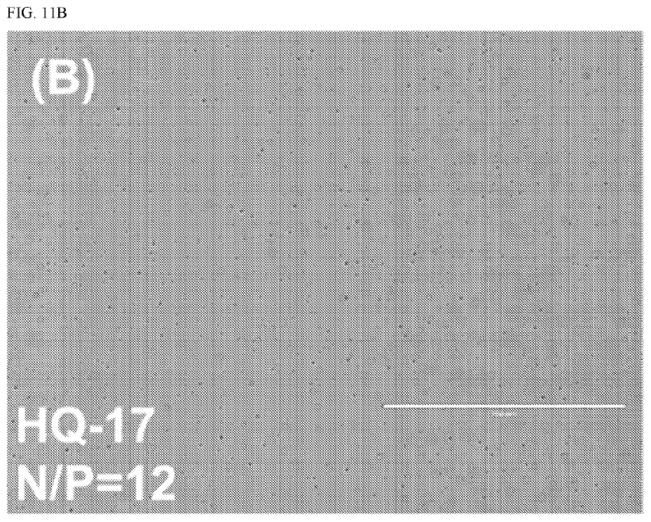

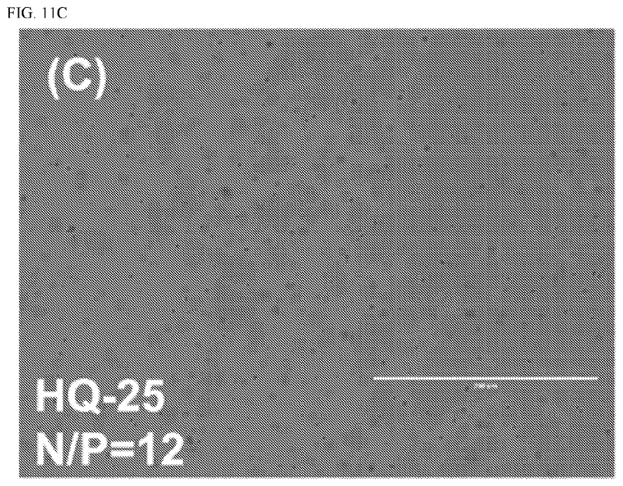

When used as a transfection agent, the electrostatic interactions between the polymer and oligonucleotide in a polyplex as well as the endosomal escape of polyplexes is at least partially influenced by the protonation state of the polymer. In quinine, there are two nitrogen centers able to be protonated, one at the quinuclidine group (pKₐ=8.5) and the other at the quinoline ring (pKₐ-4.1). Consistent with our previous work, we have studied the protonation state of the quinuclidine nitrogen center of the HQ-X polymers since that nitrogen is protonatable at physiological pH. To understand how polymer composition influences the protonation state of the polymers in aqueous solution, the pKₐ and the Hill coefficient (n_{Hill}) for the monomer as well as the HQ-X polymers (made via RAFT polymerization) were determined using potentiometric titration (see Example). Compared to HQ (pKₐ=8.3), the HQ-X polymers had lower pKₐs (≤7.0) (Table 1A, FIGS. 10A-10J). Such depression of pKa from monomer to polymer may be due to electrostatic repulsion among amine groups in proximity. The pKa of the polymer decreases as the mole percentage of HQ in the polymer chain increases, likely due to increased interactions among the HQ repeat units. The low pKₐ of HQ-X polymers suggests that only a small fraction of the amines are protonated at a pH of 7.4. The protonated amines are available for electrostatic interaction with oligonucleotides when forming a transfection agent complex (a polyplex). The reserves of unprotonated amines may act like a proton sponge, promoting endosomal escape of the polyplex to aid in delivery of the oligonucleotide to the cell and/or the nuclease of the cell. Additionally, is thought that the higher amount of HQ may increase polymer-oligonucleotide binding through enhanced intercalation.

Other than the pKₐ, the significance of cooperativity of protonation in the polymers may affect transfection efficiency of polyplexes formed using the polymers of the present disclosure. The Hill coefficient (n_{Hill}) is a measure of cooperativity in the protonation-deprotonation process. Polymer hydrophobicity has been shown to influence n_{Hill}. The Hill coefficient was determined for HQ-X polymers (made via RAFT polymerization). Similar to pKₐ, it was observed that the value of n_{Hill} increases with the increase in the mole percentage of HQ (Table 1A, FIGS. 10A-10J). The aqueous solubility of the polymers decreases with the increase in the mole percentage of HQ (c log P of HQ=4.21). Quinine is also known to interact with itself at higher concentrations and the same can be expected for HQ when it is incorporated into the polymer chain. The hydrophobic nature of HQ and the self-interactions among HQ repeat units may explain the increase in num with the increase in the percentage of HQ in the polymer chain. It has been suggested in the previous reports that polymers with higher n_{Hill} are capable of releasing their payload rapidly inside the cell cytosol in comparison to the polymers possessing lower n_{Hill} and therefore have higher gene delivery efficiency. Based on those previous findings, it was expected that the efficiency of delivering oligonucleotides into the cell nucleus to gradually increase with the increase in the percentage of IIQ in the polymer chain. However, as explained later, this was not the case for polyplexes formed from HQ-X polymers.

In another aspect, the present disclosure describes a transfection agent. A transfection agent is a material that can complex with an oligonucleotide to facilitate the transfer the oligonucleotide from the extracellular space across the cell membrane into the intracellular space. A transfection agent may be used to facilitate transfection. Transfection is the process of introducing oligonucleotides into a cell using nonviral methods. A cell that has successfully undergone transfection, that is, the oligonucleotide is located within the cell (cytoplasm or nuclease), is a transfected cell. The complex of the oligonucleotide and the transfection agent is a transfection complex, also referred to as a polyplex. In the present disclosure the transfection agent includes one or more of the quinoline-containing monomer polymers as described elsewhere herein. The polymer can complex with an oligonucleotide to from a transfection complex also termed a polyplex, a polyplex complex, or a polyplex transfection complex. The oligonucleotide associates with (e.g., binds to) the polymer via electrostatic interactions between the negatively charged oligonucleotide backbone and the protonated amines (e.g., the quinuclidine nitrogen of the polymer). Additionally, intercalation of the quinoline-containing monomers and the oligonucleotide may contribute to binding. The polyplex facilitates transport of the oligonucleotide across the cell membrane.

The ratio of phosphate groups of the oligonucleotide to the amine group (N/P ratio) of the polymer (e.g., quinuclidine nitrogen or) may affect the efficiency of the polyplex as a transfection agent complex. As used herein, the N/P ration refers to the N/P ratio of a single polyplex or the average N/P ration of a plurality of polyplexes. In one or more embodiments the polyplex N/P ratio is 1 or greater, 3 or greater, 5 or greater, 6 or greater, 8 or greater, 10 or greater, 12 or greater, 14 or greater, 16 or greater, 18 or greater, 20 or greater, 25 or greater, or 30 or greater. In one or more embodiments the polyplex N/P ratio is 40 or less, 35 or less, 30 or less, 25 or less, 20 or less, 18 or less, 16 or less, 14 or less, 12 or less, 10 or less, 8 or less, 6 or less, 5 or less, or 3 or less. In one or more embodiments the polyplex N/P ratio is 1 to 40, 1 to 35, 1 to 30, 1 to 25, 1 to 20, 1 to 18, 1 to 16, 1 to 14, 1 to 12, 1 to 12, 1 to 10, 1 to 8, 1 to 6, 1 to 5, or 1 to 3. In one or more embodiments the polyplex N/P ratio is 3 to 40, 3 to 35, 3 to 30, 3 to 25, 3 to 20, 3 to 18, 3 to 16, 3 to 14, 3 to 12, 3 to 12, 3 to 10, 3 to 8, 3 to 6, or 3 to 5. In one or more embodiments the polyplex N/P ratio is 5 to 40, 5 to 35, 5 to 30, 5 to 25, 5 to 20, 5 to 18, 5 to 16, 5 to 14, 5 to 12, 5 to 12, 5 to 10, 5 to 8, or 5 to 6. In one or more embodiments the polyplex N/P ratio is 6 to 40, 6 to 36, 6 to 30, 6 to 26, 6 to 20, 6 to 18, 6 to 16, 6 to 14, 6 to 12, 6 to 12, 6 to 10, or 6 to 8. In one or more embodiments the polyplex N/P ratio is 8 to 40, 8 to 36, 8 to 30, 8 to 26, 8 to 20, 8 to 18, 8 to 16, 8 to 14, 8 to 12, 8 to 12, or 8 to 10. In one or more embodiments the polyplex N/P ratio is 10 to 40, 10 to 36, 10 to 30, 10 to 26, 10 to 20, 10 to 18, 10 to 16, 10 to 14, or 10 to 12. In one or more embodiments the polyplex N/P ratio is 12 to 40, 12 to 36, 12 to 30, 12 to 26, 12 to 20, 12 to 18, 12 to 16, or 12 to 14. In one or more embodiments the polyplex N/P ratio is 14 to 40, 14 to 36, 14 to 30, 14 to 26, 14 to 20, 14 to 18, or 14 to 16. In one or more embodiments the polyplex N/P ratio is 16 to 40, 16 to 36, 16 to 30, 16 to 26, 16 to 20, or 16 to 18. In one or more embodiments the polyplex N/P ratio is 18 to 40, 18 to 36, 18 to 30, 18 to 26, or 18 to 20. In one or more embodiments the polyplex N/P ratio is 20 to 40, 20 to 36, 20 to 30, or 20 to 26. In one or more embodiments the polyplex N/P ratio is 25 to 40, 25 to 36, 25 to 30, 30 to 36, or 35 to 40.

The size of the polyplex may affect the transfection efficiency of the polyplex. For example, the size of the polyplex may influence the internalization pathway used by the cell during transfection. As used herein, the size of a polyplex transfection complex refers to the hydrodynamic diameter (dₕ) of a polyplex or the average hydrodynamic diameter of a plurality of polyplexes. The hydrodynamic diameter may be measured using dynamic light scattering, for example (see Example). A polyplex (or plurality of polyplexes) may have an undiluted dn and a treatment dh. The undiluted dn refers to the dn of a polyplex (or a plurality of polyplexes) in the mixture in which they are formed prior to any dilution.

In some cases, the medium in which polyplexes are initially formed may be unsuitable for live cells. For example, polyplexes may be formed in a solution at an acidic pH (e.g., a pH of 1 to 5). Direct exposure of the cells to such as solution may stress and/or kill the cells. Polyplexes may also be formed in pure water without additives such as salts. Such solutions rupture the cells due to difference in osmotic pressure inside and outside the cells. Additionally, polyplexes may be formed using a high concentration of the polymer and/or oligonucleotide. The resultant polyplex solution may include a concentration of polyplexes that is too high for exposure to cells. As such, in some embodiments, prior to exposure to cells, an undiluted polyplex mixture is diluted with a dilution medium. The dilution factor, the dilution medium, and the pH of the medium are chosen so as to minimize cell death upon exposure to the polyplexes. In some cases, when a mixture of polyplexes are diluted, the polyplexes may aggregate to form complexes having a larger dn than prior to dilution. As used herein, the treatment dn also referred to as an aggregate dₕ, refers to the dn of the polyplex (or plurality of polyplexes) that are used to treat (e.g., transfect cells). In one or more embodiments, the undiluted dₕ and the treatment dn are the same. In other embodiments, the undiluted dh and the treatment dn are different. In some such embodiments, the undiluted dₕ is smaller than the treatment dₕ. Polyplexes that have been diluted and are ready for exposure to cells may be referred to as diluted polyplexes or aggregated polyplexes.

In one or more embodiments, the undiluted dh and/or the treatment dn is 20 nm or greater, 40 nm or greater, 50 nm or greater, 60 nm or greater, 70 nm or greater, 80 nm or greater, 90 nm or greater, 100 nm or greater, 200 nm or greater, 300 nm or greater, 400 nm or greater, 500 nm or greater, 600 nm or greater, 700 nm or greater, 800 nm or greater, 900 nm or greater, 1000 nm or greater, 1100 nm or greater, 1200 nm or greater, 1300 nm or greater, 1400 nm or greater, 1500 nm or greater, 1600 nm or greater, 1700 nm or greater, 1800 nm or greater, or 1900 nm or greater. In one or more embodiments, the undiluted dh and/or the treatment dh is 2000 nm or less, 1900 nm or less, 1800 nm or less, 1700 nm or less, 1600 nm or less, 1500 nm or less, 1400 nm or less, 1300 nm or less, 1200 nm or less, 1100 nm or less, 1000 nm or less, 900 nm or less, 800 nm or less, 700 nm or less, 600 nm or less, 500 nm or less, 400 nm or less 300 nm or less, 200 nm or less, 100 nm or less, 90 nm or less, 80 nm or less, 70 nm or less, 60 nm or less, 50 nm or less, or 40 nm or less. In one or more embodiments, the undiluted dh and/or the treatment dh is 20 nm to 2000 nm, 20 nm to 1900 nm, 20 nm to 1800 nm, 20 nm to 1700 nm, 20 nm to 1600 nm, 20 nm to 1500 nm, 20 nm to 1400 nm, 20 nm to 1300 nm, 20 nm to 1200 nm, 20 nm to 1100 nm, 20 nm to 1000 nm, 20 nm to 900 nm, 20 nm to 800 nm, 20 nm to 700 nm, 20 nm to 600 nm, 20 nm to 500 nm, 20 nm to 400 nm, 20 nm to 300 nm, 20 nm to 200 nm, 20 nm to 100 nm, 20 nm to 90 nm, 20 nm to 80 nm, 20 nm to 60 nm, or 20 nm to 50 nm. In one or more embodiments, the undiluted dh and/or the treatment dn is 20 nm to 200 nm, 20 nm to 100 nm, 40 nm to 200 nm, 40 nm to 100 nm, 40 nm to 80 nm, 40 nm to 60 nm, 100 nm to 1500 nm or greater, 500 nm to 1500 nm or greater, 1000 nm to 1500 nm or greater, 300 nm to 900 nm, 300 nm to 800 nm, 400 nm to 800 nm, or 400 nm to 600 nm.

The polymers present disclosure may be used as a transfection agent to transfect cells with a variety of oligonucleotides. In such embodiments, the polymer or plurality of polymers are complexed with an oligonucleotide or plurality of oligonucleotides to form a polyplex. As used herein, the term “oligonucleotide” refers to a polymer of two or more nucleotides. An oligonucleotide may be single stranded; that is, include a single polymer of nucleotides or double stranded; that is, include two polymers of nucleotides that are at least partially hybridized to each other. An oligonucleotide may include deoxyribonucleotides, ribonucleotides, or both. The oligonucleotide may include non-canonical deoxyribonucleotides, non-canonical ribonucleotides, or both. Examples of oligonucleotides that may be transfected into cells using the polymers of the present disclosure include, but are not limited to, plasmid DNA (pDNA), messenger RNA, antisense oligonucleotides, small interfering RNA, micro-RNA, guide RNA, Cas9-sgRNa complexes, aptamers, derivatives thereof, and combinations thereof. In one or more embodiments, the polymers of the present disclosure may be used as a transfection agent for the transfection of pDNA into a cell.

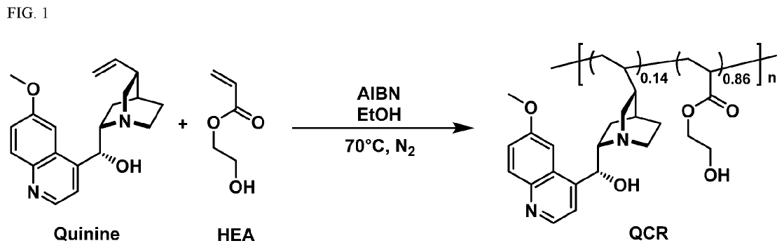

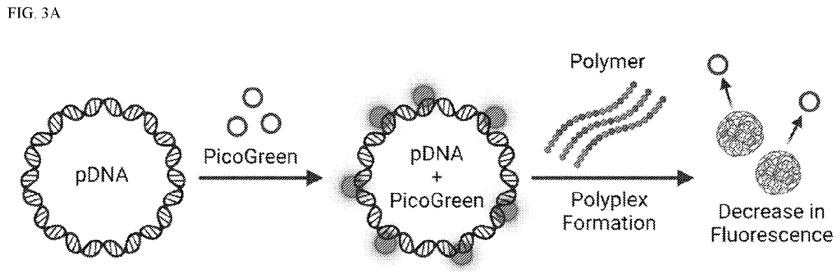

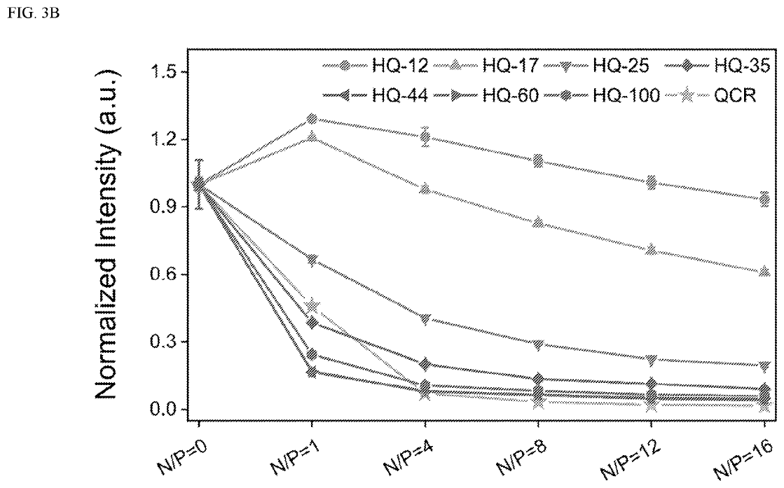

The ability of the HQ-X polymers (Table 1A; made via RAFT polymerization) to bind with and compact pDNA into polyplexes was evaluated using a dye exclusion assay (see Example), at different N/P ratios, where N stands for the number of moles of quinuclidine nitrogen atoms and P stands for the number of moles of phosphate groups from pDNA in the solution. A copolymer (QCR; FIG. 1) of quine and HEA having 14 mol-% quinine was also tested. In this assay (see FIG. 3A), pDNA was first mixed with PICOGREEN, a molecule that is fluorescent when intercalated in the pDNA (λₑₓ=485 nm, λₑₘ=528 nm). Subsequently, the polymer solutions were added to the pDNA-PICOGREEN mixture at different N/P ratios to allow the formation of polyplexes. The decrease in the fluorescence intensity, resulting from the exclusion of PICOGREEN from pDNA due to competitive binding and pDNA compaction, was measured (FIGS. 3A and 3B). For all the polymers, increasing the N/P ratio resulted in a decrease in fluorescence intensity, indicating stronger binding between the polymer and pDNA at higher N/P. At all N/P ratios, polymer-pDNA binding increased monotonically with the increase in mole percentage of HQ. QCR, with only 14 mol-% quinine incorporation, shows stronger binding to pDNA compared to HQ-12 and HQ-17 despite the comparable molar composition of the polymers. QCR shows stronger binding than HQ-25 (FIG. 3B). This difference in binding strength may be attributed to the differences in monomer structure and orientation of the side chain in polymers. Unlike QCR, HQ-X polymers do not possess the hydroxyl group that can participate in hydrogen bonding with the pDNA (FIGS. 1 and 2A). Previous work using molecular dynamic simulations has shown that the hydroxyl group in quinine can interact with nucleobases in DNA through hydrogen bonding interactions. As such, the lack of the hydroxyl group on the HQ repeat unit, in contrast to quinine, may result in relatively weaker binding with pDNA compared to QCR, at comparable molar compositions.

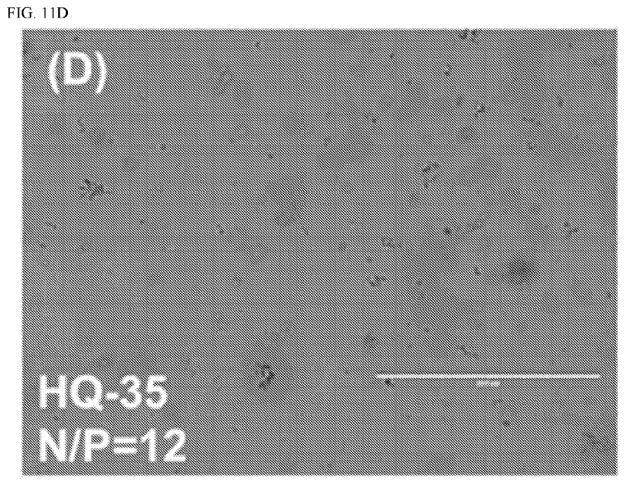

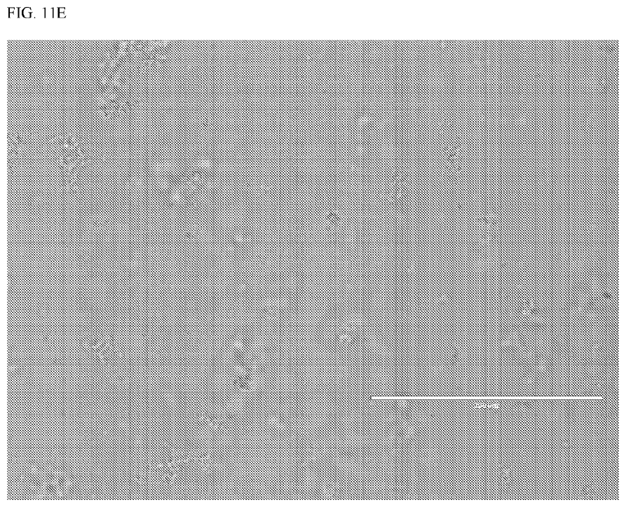

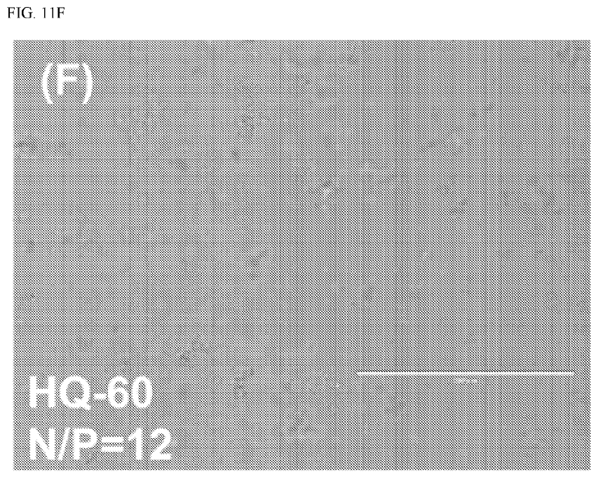

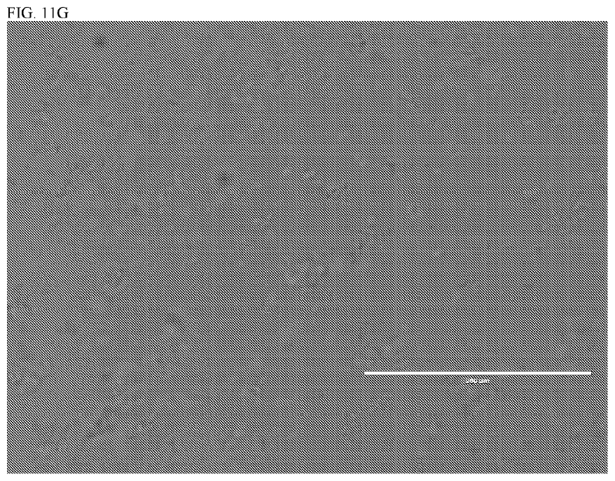

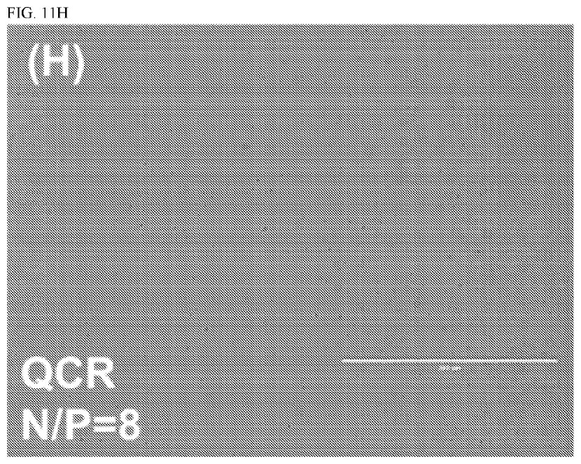

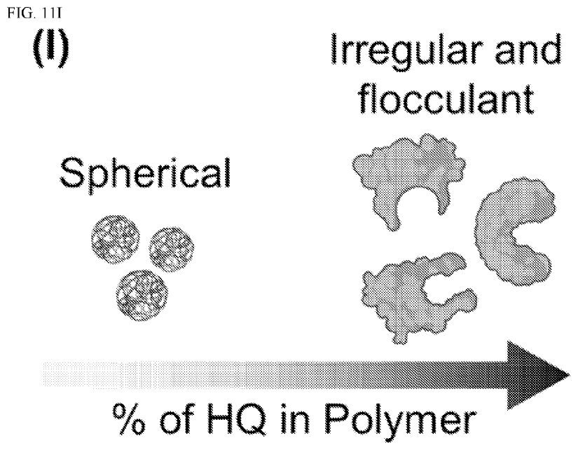

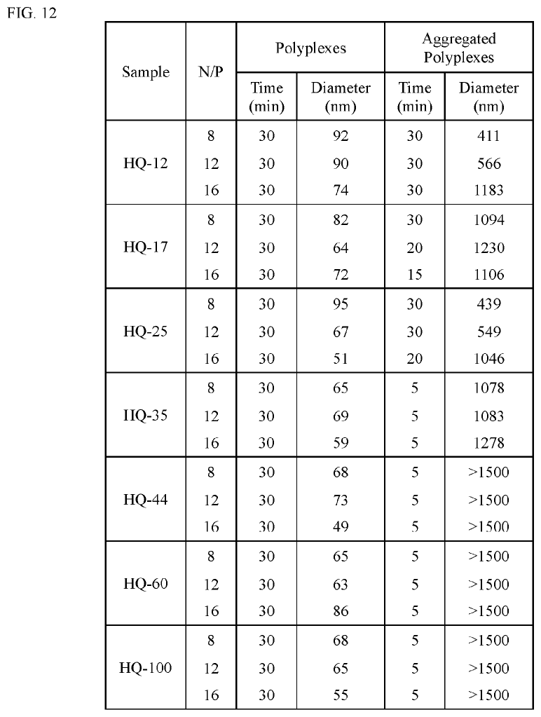

Polyplex size may impact oligonucleotide delivery efficiency as cell membrane adhesion, cellular internalization, and intracellular trafficking of polyplexes may be size dependent. QCR polymers bind with pDNA to initially form polyplexes that are 80 nm to 200 nm in hydrodynamic diameter (dh). Upon dilution with serum-free cell culture media, polyplexes that include QCR form large aggregates (dₕ>1000 nm), likely due to the hydrophobicity of quinine at pH ~7.4. The large size of the polyplexes was advantageous for in vitro transfection as the polyplex particles settled on the cells faster. Using dynamic light scattering (DLS), it was observed that all HQ-X polymers (made via RAFT polymerization) and FRP HQ-X polymers (made via FRP) form polyplex particles by binding with pDNA and the particle sizes (dₕ) range between 50 to 100 nm (FIG. 12 and FIG. 28C; in FIG. 28C polymer+DNA is polyplexes, polymer+DNA+FLUOROBRITE is diluted polyplexes (aggregated polyplexes), and polymer+FLUOROBRITE is a control with no DNA). Dilution of the polyplex solution with serum-free media (FLUOROBRITE DMEM), results in polyplex particle aggregation over time. Some aggregates grew up to 1000 nm or larger, in diameter, within 30 minutes (FIGS. 11A-11I; FIG. 12; and FIG. 28C). In general, for the HQ-X series (made via RAFT polymerization) a higher mole percentage of HQ in the polymer leads to faster aggregation and larger polyplex particles. At mole percentages of HQ higher than 35%, the polyplexes show a high propensity for aggregation pushing them beyond the scope of DLS measurements (dₕ>1500 nm). Since internalization of the polyplex particles may be a size-dependent phenomenon, it was expected that the largest aggregates would have a low transfection efficiency due to poor internalization. However, the results from cellular uptake experiments discussed herein show that polymers producing the largest aggregates can deliver pDNA inside the cells.

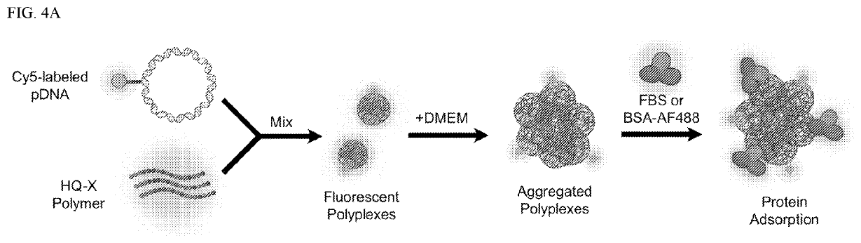

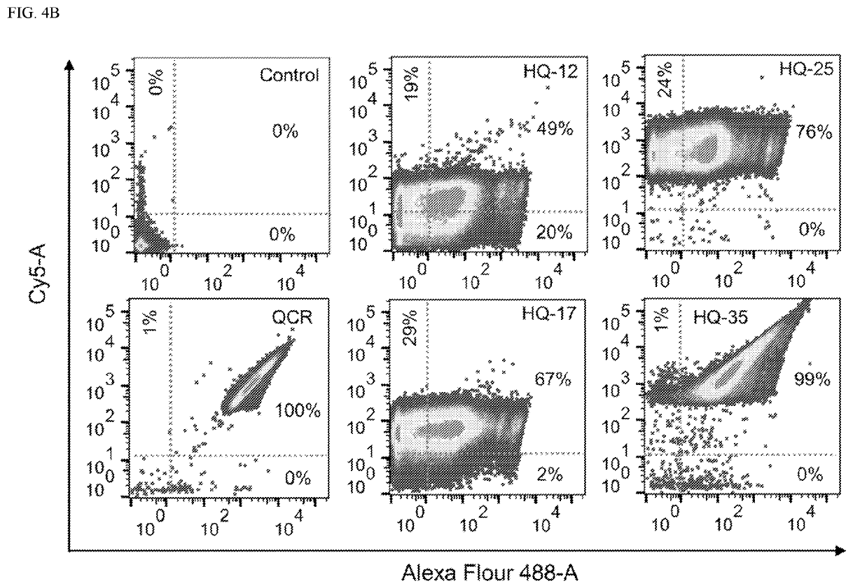

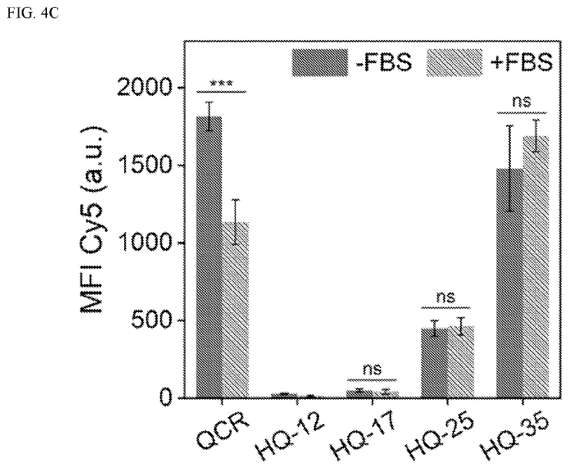

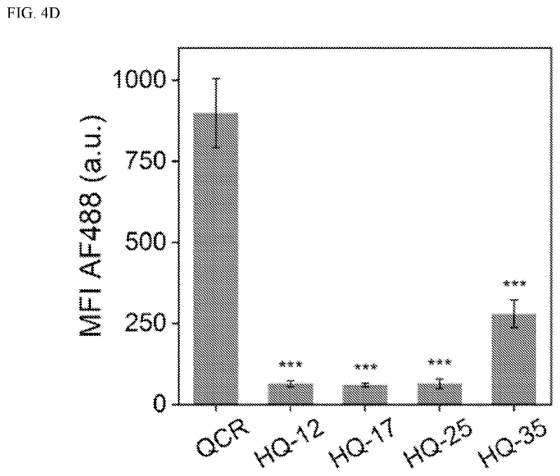

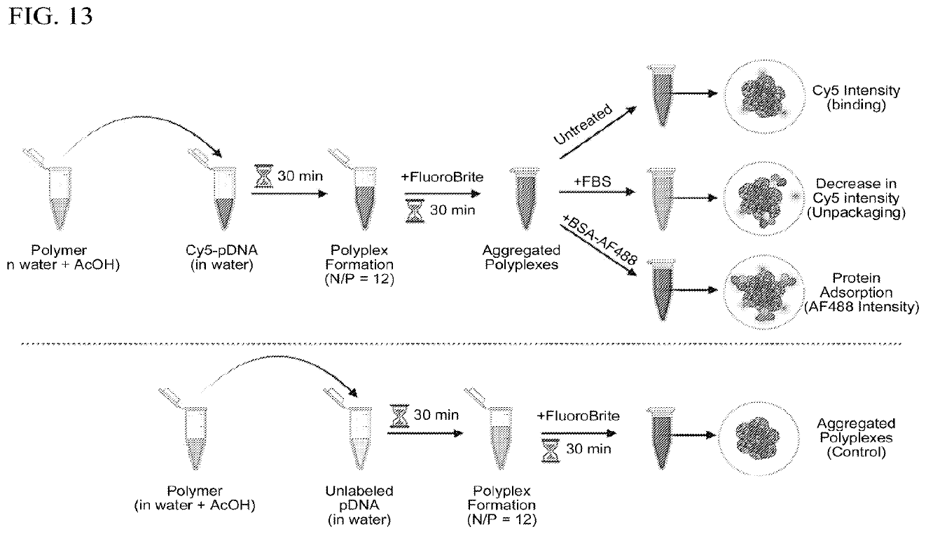

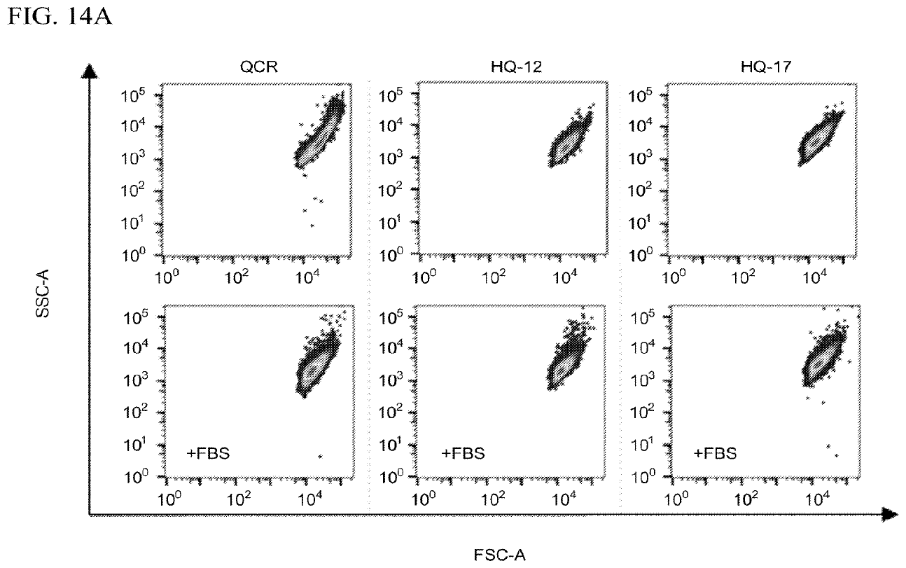

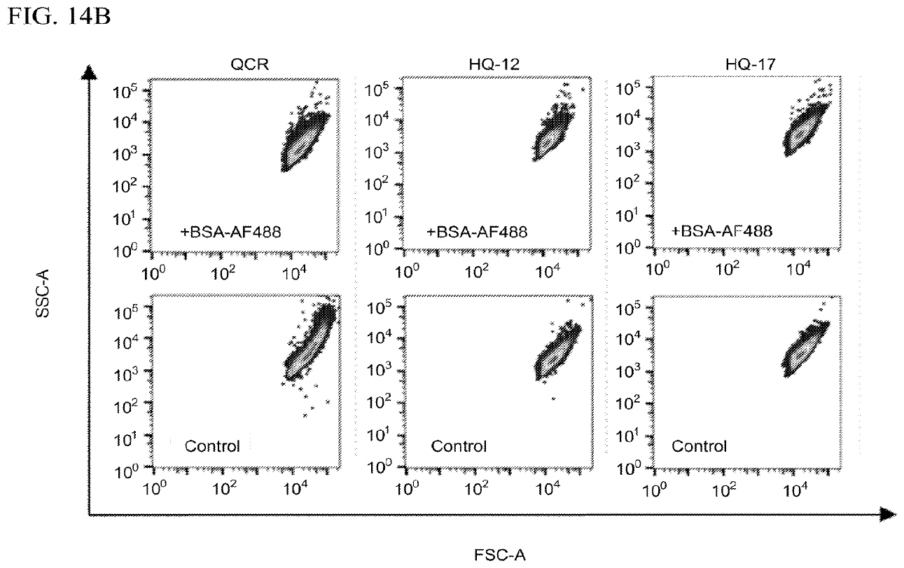

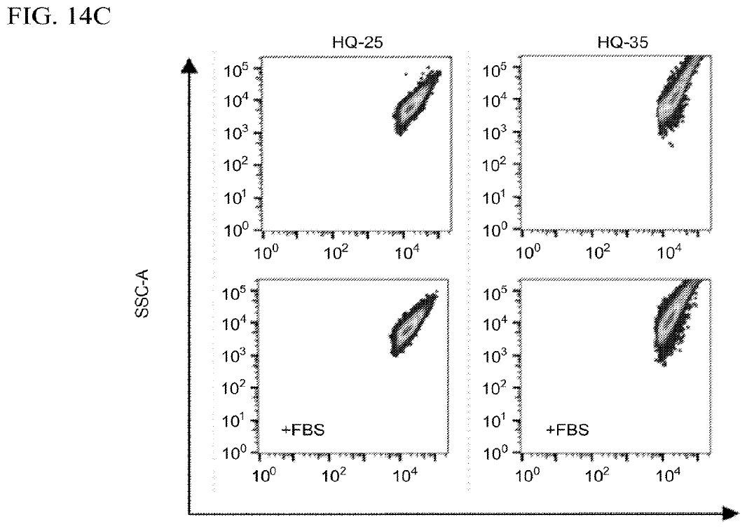

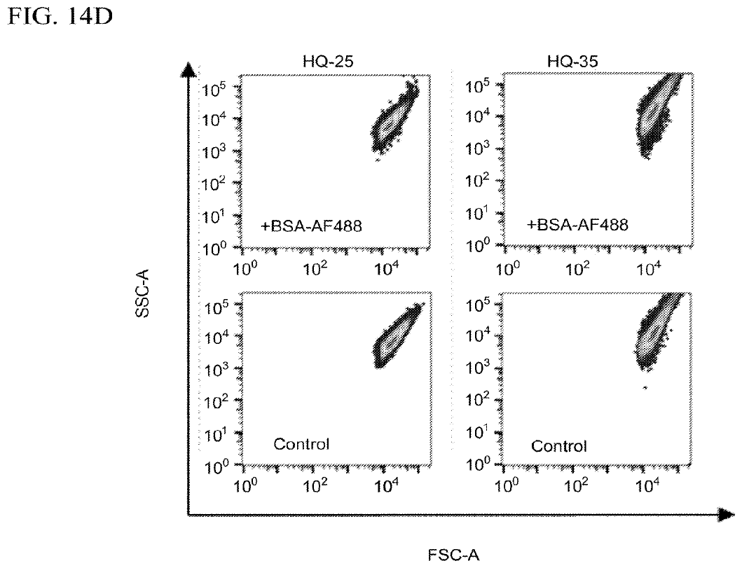

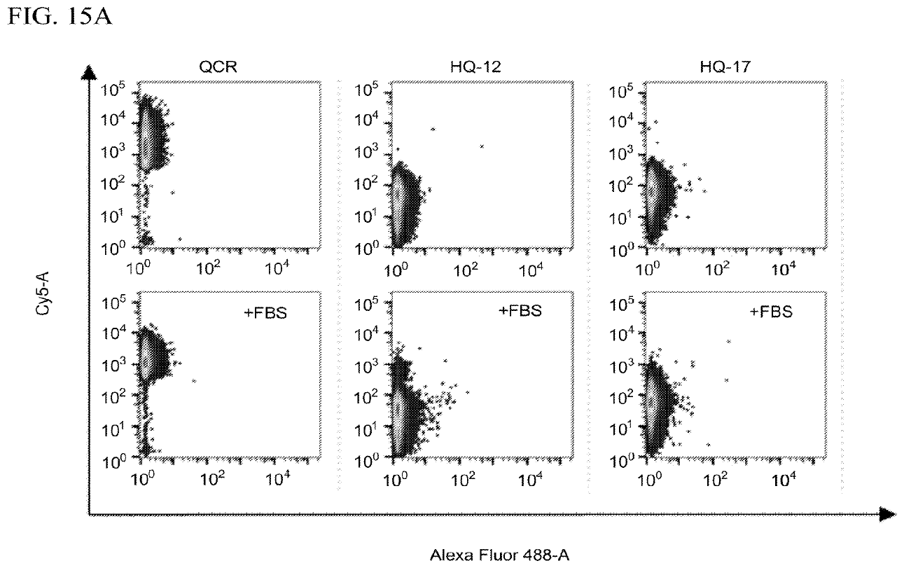

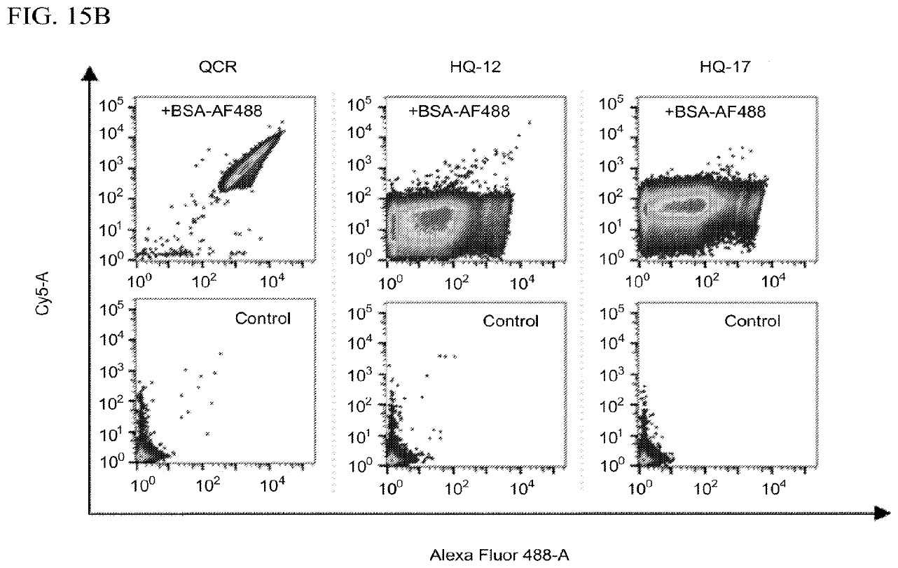

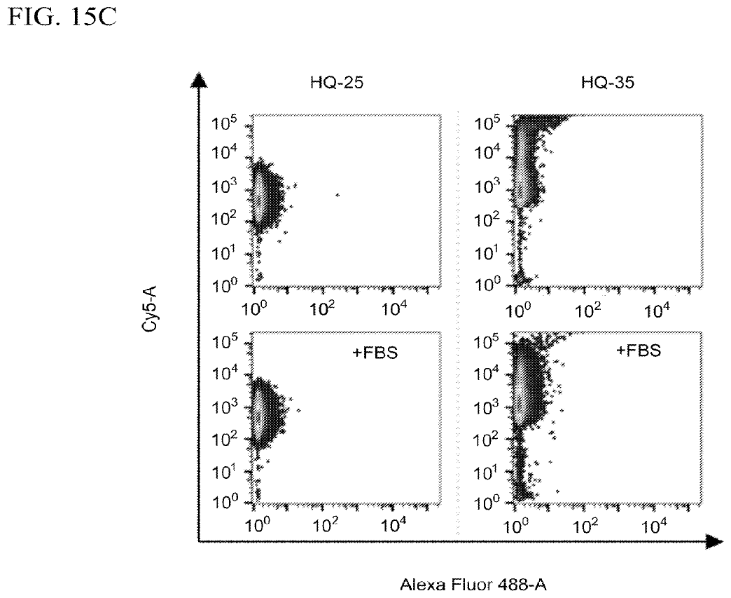

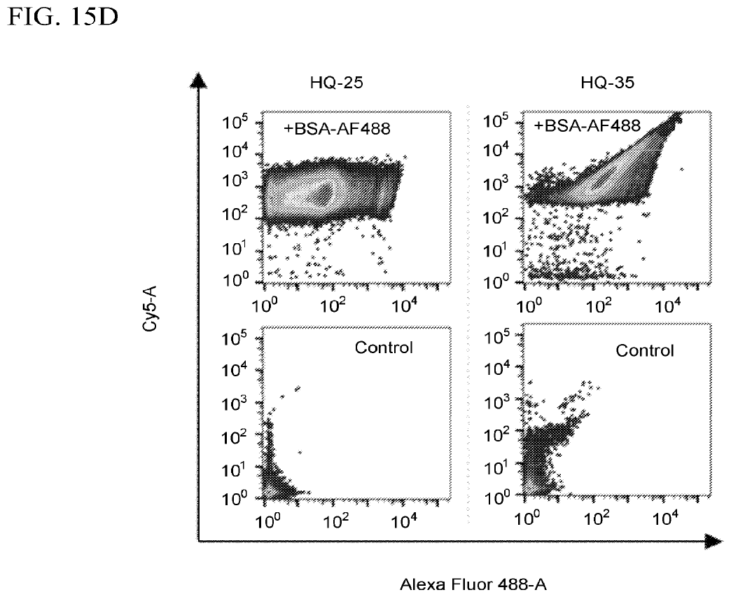

The characteristics of the aggregated polyplex particles formed with HQ-12, HQ-17, HQ-25, HQ-35 (HQ-X series made via RAFT polymerization; see Table 1A) as well as QCR were studied in more detail using flow cytometry (see Example) since their sizes ranged above the lower limit of detection (diameter≥500 nm) for the flow cytometer (FIG. 11A-11I and FIG. 12). HQ-44, HQ-60, and HQ-100 (see Table 1A) could not be studied via this method as they formed large nonuniform flocculant aggregates. Fluorescently labeled polyplexes were formed by mixing Cy5-labeled pDNA with the polymers, at N/P=16. Next, aggregation of the polyplexes was induced by dilution with FLUOROBRITE DMEM (see FIG. 4A, FIG. 13, FIGS. 14-14D, and FIGS. 15A-15D). The Cy5 fluorescence intensity (λₑₓ=651 nm, λₑₘ=670 nm) was used as a parameter to compare the amount of pDNA present in each aggregated polyplex particle. The average Cy5 fluorescence intensity per particle increased with the increase in mole percentage of HQ in the polymer chain (FIGS. 4B and 4C) implying a higher amount of pDNA per polyplex particle and corroborating the results from the dye exclusion assay (FIG. 3B). Additionally, aggregated polyplexes of QCR showed about 35-fold higher Cy5 fluorescence intensity than that of HQ-17 despite having a similar polymer composition. This indicates that the quinine repeat unit has a stronger affinity towards pDNA than the HQ repeat unit.

For engineered nanoparticles, protein adsorption and the resultant protein corona affect their biological fate. Serum proteins are responsible for unpackaging of oligonucleotides from polyplexes of QCR. To compare the influence of polymer composition on protein adsorption and oligonucleotide unpackaging, in the second part of the flow cytometry experiment, the HQ-X series aggregated polyplexes were incubated with fetal bovine serum (FBS) and the changes in the Cy5 fluorescence due to the presence of serum proteins were measured (see Example). Introduction of FBS in the polyplex solution led to about 35% decrease in the Cy5 fluorescence intensity from the polyplexes formed using QCR. This indicates the protein-mediated release of pDNA from polyplexes. In contrast, polyplexes formed with HQ-X showed different characteristics. Specifically, the polyplex associated Cy5 fluorescence intensity did not change significantly upon the introduction of FBS to the system (FIGS. 4B and 4C). This suggests that compared to QCR, polyplexes formed from HQ-X polymers are less susceptible to protein-mediated unpackaging.

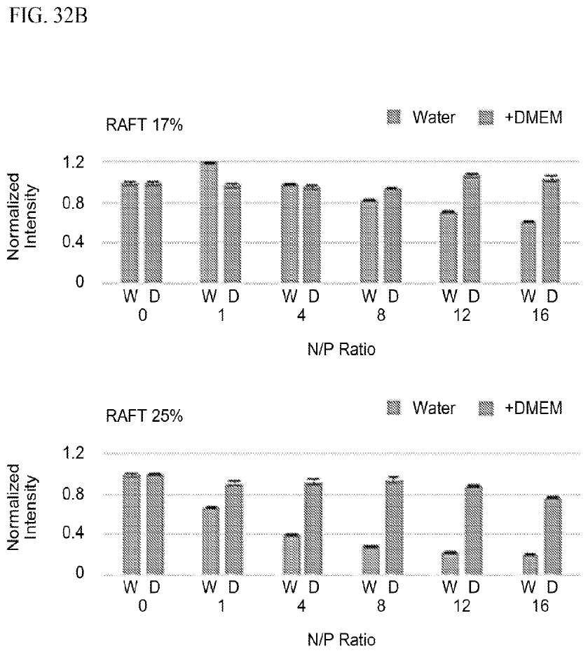

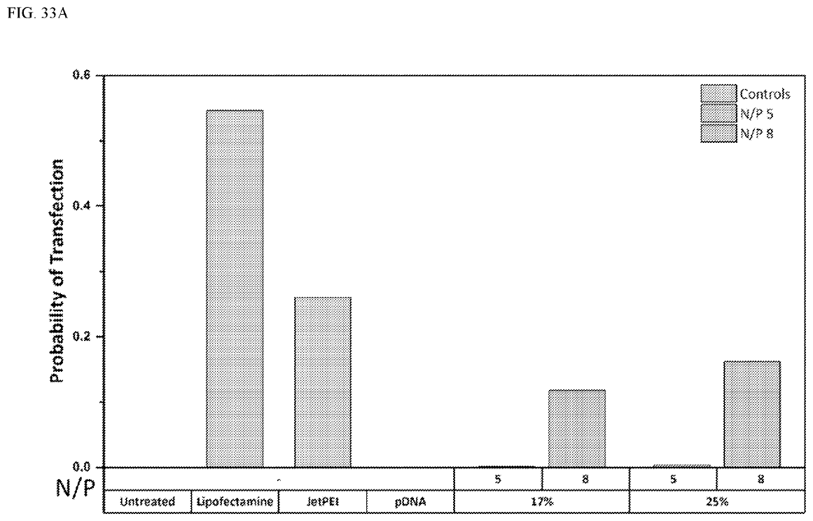

In a similar experiment, DMEM was added to polyplex formed from HQ-17 and HQ-25 (from the HQ-X) series and FRP HQ-17 and FRP HQ-25 and Cy5 labelled pDNA. FIGS. 32A and 32B show a comparison of the Cy5 intensity when water of DMEM was added to the polyplex solutions. The RAFT series (HQ-X) and FRP series (FRP HQ-X) show similar results.

To compare the extent of protein adsorption on the polyplexes, the aggregated polyplexes formed from HQ-X polymers were incubated with ALEXAFLUOR 488-labeled bovine serum albumin (BSA-AF488) instead of FBS and maintained the same protein concentration as the previous experiment. The fluorescence intensity of ALEXAFLUOR 488 (λex=490 nm, λem=525 nm) associated with the aggregated polyplexes was used as an indicator for the amount of protein adsorption (see Example). The geometric mean fluorescence intensities from the polyplex particles indicated that polyplexes formed with HQ-X polymers adsorb less protein relative to polyplexes formed using QCR (more than a 3-fold difference in mean fluorescence intensity) (FIGS. 4B and 4D). This explains the difference observed for changes in Cy5 intensity after the addition of FBS to the polyplexes. Specifically, compared to polyplexes formed from QCR, a significantly less amount of serum protein is adsorbed onto the polyplexes formed from HQ-12 to HQ-35 polymers and consequentially the degree of pDNA unpackaging is also lower (FIG. 4C). In other words, polyplexes formed with HQ-X polymers have a lower propensity for protein adsorption and therefore exhibit less unpackaging in protein-rich media compared to polyplexes formed with QCR. This polyplex flow cytometry workflow enabled qualitative comparison of different polyplex formulations in three parameters: (i) pDNA loading per polyplex, (ii) adsorption of serum proteins on the polyplexes, and (iii) extent of payload unpackaging that is facilitated by serum proteins. Similar to the dye exclusion assay, these results indicate that polymer-pDNA binding strength increases with the increase in the mole percentage of HQ in polymer chain and that for similar molar composition, QCR binds to pDNA more strongly than HQ variants. Additionally, polyplexes formed from HQ-X adsorb less protein than polyplexes formed from QCR, and HQ-X polymers are notably more resilient from protein-mediated payload release.

In another aspect, the present disclosure describes a transfection composition. The composition may be a transfection composition. The transfection composition includes a transfection complex otherwise termed a polyplex or a plurality of transfection complexes. The transfection complex includes a quinoline-containing polymer of the present disclosure and an oligonucleotide. The transfection composition further includes a transfection medium. The size of the polyplex or plurality of polyplexes in a transfection composition is the treatment dh (as described elsewhere herein). The treatment dn may be any treatment dn as described herein.

The transfection medium may be any suitable medium for contact with cells. In some embodiments, the medium comprises water. In one or more embodiments, the medium includes salts and or agents or compounds that promote cell health and/or growth. In one or more embodiments, the medium is a cell growth medium, such as, for example, Dulbecco's Modified Eagle Medium (DMEM), Minimum Essential Medium (MIEM), RPMI 1640 Medium, Opti-MEM™ I. In one or more embodiments, the medium includes proteins. In other embodiments, the medium does not include proteins. In one or more embodiments, the medium is serum free. In other embodiments, the medium includes serum.

The pH of the transfection composition may vary. In one or more embodiments, the pH of the medium is 2 or greater, 3 or greater, 4 or greater, 5 or greater, 6 or greater, 7 or greater, or 8 or greater. In one or more embodiments, the pH of the medium is 9 or less, 8 or less, 7 or less, 6 or less, 5 or less, 4 or less, or 3 or less. In some embodiments, the pH of the medium is 4 to 8, 5 to 8, 6 to 8, or 7 to 8.

In another aspect, the present disclosure describes a method. The method includes forming a polyplex transfection complex. The method generally includes mixing a polymer that includes a quinoline-containing monomer with an oligonucleotide to create a first mixture. The method also includes incubating the first mixture for a period of time to form a second mixture comprising the polyplex transfection complex. In some embodiments, the second mixture is a transfection composition that can be used to treat cells.

The absolute concentration of the quinoline-containing polymer and the absolute concentration of the oligonucleotide in the first mixture may vary depending at least in part on the desired N/P ratio, the identity and properties of the quinoline-containing polymer, the identity and properties of the oligonucleotide, the number of cells to be treated, other factors, or any combination thereof.

In one or more embodiments, the first mixture includes water. In one or more embodiments, the first mixture includes an aqueous solution of water and an acid. The acid is used to adjust the pH to a value of 1 to 5. A low pH may be beneficial to polyplex formation as a low pH may increase the probability that the amines of the polymer are protonated. Protonation of the amines gives the amines a positive charge which may increase electrostatic interactions between the polymer and the negatively charged backbone of the oligonucleotide. Any suitable acid or buffer may be included. Examples of acids include, but are not limited to, citric acid, acetic acid, hydrochloric acid, formic acid, lactic acid, uric acid, malic acid, tartaric acid, sulfuric acid, and the like. Examples of buffer include sodium acetate buffer, chloroacetate buffer, and the like.

The length of first incubation period of time may affect the transfection efficiency of the polyplex. In one or more embodiments, the first period of time is 1 minute or greater, 15 minutes or greater, 20 minutes or greater, 25 minutes or greater, 30 minutes or greater, 40 min or greater, 1 hour or greater, or 5 hours or greater. In one or more embodiments, the first period of time is 5 hours or less, 1 hour or less, 40 minutes or less, 35 minutes or less, 30 minutes or less, 25 minutes or less, 20 minutes or less, or 15 minutes or less. In one or more embodiments, the first period of time is 15 minutes to 40 minutes, 15 minutes to 35 minutes, 15 minutes to 30 minutes, 15 minutes to 25 minutes, or 15 minutes to 20 minutes. In one or more embodiments, the first period of time is 20 minutes to 40 minutes, 20 minutes to 35 minutes, 20 minutes to 30 minutes, or 20 minutes to 25 minutes. In one or more embodiments, the first period of time is 25 minutes to 40 minutes, 25 minutes to 35 minutes, or 25 minutes to 30 minutes. In one or more embodiments, the first period of time is 30 minutes to 40 minutes or 30 minutes to 35 minutes. In one or more embodiments, the first period of time is 25 minutes to 35 minutes.

In one or more embodiments, the method further includes diluting the second mixture with a dilution medium to create a third mixture. In some embodiments, the third mixture is a transfection composition that can be used to treat cells. In such embodiments, the dilution medium is a transfection medium. In one or more embodiments, the method further includes incubating the third mixture for second period of time to form a transfection composition. In some such embodiments, the incubation of the third mixture allows for the aggregation of polyplexes to form polyplex particles having a larger dn than in the second mixture.

The second mixture may be diluted with an amount of a medium such that the concentration of the polyplexes in the third mixture is half is 99% or less, 90% or less, 80% or less, 70% or less, 60% or less, 50% or less, 40% or less, 30% or less, 20% or less, 10% or less, 5% or less, 1% or less 0.1% or less, 0.01% or less, 0.001% or less, 0.0001% or less of the concentration of the polyplexes in the second mixture. In one or more embodiments, the second mixture may be diluted with an amount of a medium such that the concentration of the polyplexes in the third mixture is 0.0001% or greater, 0.001% or greater, 0.01% or greater, 0.1% or greater, 1% or greater, 5% or greater, 10% or greater, 20% or greater, 30% or greater, 40% or greater, 50% or greater, 60% or greater, 70% or greater, 80% or greater, or 90% or greater of the concentration of the polyplexes in the second mixture. In one or more embodiments, the second mixture may be diluted with an amount of a medium such that the concentration of the polyplexes is 10% to 90%, 20% to 80%, or 40% to 60% of the concentration of the polyplexes in the second mixture.

The length of the second period of time may affect the efficiency of transfection. In one or more embodiments, the second period of time is 1 min or greater, 15 minutes or greater, 20 minutes or greater, 25 minutes or greater, or 30 minutes or greater, 40 minutes or greater, 1 hour or greater, or 5 hours or greater. In one or more embodiments, the second period of time is 5 hours or less, 1 hour or less, 40 minutes or less, 35 minutes or less, 30 minutes or less, 25 minutes or less, 20 minutes or less, or 15 minutes or less. In one or more embodiments, the second period of time is 15 minutes to 40 minutes, 15 minutes to 35 minutes, 15 minutes to 30 minutes, 15 minutes to 25 minutes, or 15 minutes to 20 minutes. In one or more embodiments, the second period of time is 20 minutes to 40 minutes, 20 minutes to 35 minutes, 20 minutes to 30 minutes, or 20 minutes to 25 minutes. In one or more embodiments, the second period of time is 25 minutes to 40 minutes, 25 minutes to 35 minutes, or 25 minutes to 30 minutes. In one or more embodiments, the second period of time is 30 minutes to 40 minutes or 30 minutes to 35 minutes. In one or more embodiments, the second period of time is 25 minutes to 35 minutes.

In another aspect, the present disclosure describes a method of transfecting a cell with a polyplex transfection complex of the present disclosure. In some such embodiments, the polyplex transfection complex is in a transfection composition of the present disclosure. The polyplex transfection complex and/or transfection composition may include any components as disclosed herein and may be formed according to any method disclosed herein. The method includes contacting a polyplex transfection complex or transfection composition with a cell to form a transfection mixture. In one or more embodiments, the transfection composition is the third mixture following the second incubation time from the method of forming the polyplex transfection complex described herein. In one or more embodiments, the method further incudes incubating the transfection mixture for a transfection time. In one or more embodiments, the method further includes quenching the transfection mixture.

The pH of the polyplex transfection complex or transfection composition used to treat the cells may affect the efficiency of transfection. For example, a pH that is too high or too low may kill the cells. In one or more embodiments, the pH of the polyplex or transfection composition is between 5 and 8. In one or more embodiments, the pH of polyplex or transfection composition is between 7 and 8.

Similarly, the length of the transfection time may affect the efficiency of transfection. In one or more embodiments, the transfection time is 1 minute or greater, 15 minutes or greater, 20 minutes of greater, 25 minutes or greater, or 30 minutes or greater, 40 minutes or greater, 1 hour or greater, 5 hours or greater, 12 hours or greater, 24 hours or greater, 48 hours or greater, 64 hours or greater, or 96 hours or greater. In one or more embodiments, the transfection time of time is 96 hours or less, 64 hours or less, 48 hours or less, 24 hours or less, 12 hours or less, 5 hours or less, 1 hour or less, 40 minutes of less, 35 minutes or less, 30 minutes or less, 25 minutes or less, or 20 minutes or less. In one or more embodiments, the transfection time of time is 15 minutes to 40 minutes, 15 minutes to 35 minutes, 15 minutes to 30 minutes, 15 minutes to 25 minutes, or 15 minutes to 20 minutes. In one or more embodiments, transfection time is 20 minutes to 40 minutes, 20 minutes to 35 minutes, 20 minutes to 30 minutes, or 20 minutes to 25 minutes. In one or more embodiments, the transfection time is 25 minutes to 40 minutes, 25 minutes to 35 minutes, or 25 minutes to 30 minutes. In one or more embodiments, the transfection time is 30 minutes to 40 minutes or 30 minutes to 35 minutes. In one or more embodiments, the transfection time is 25 minutes to 35 minutes.

Quenching the transfection mixture decreases the likelihood of transfection. Quenching the transfection mixture may increases cell survival during and/or after the transfection process.

In one or more embodiments, quenching the transfection mixture includes adding serum-containing media to the transfection mixture to create a quenched mixture. The serum in the serum-containing media disrupts the ability of the transfection complex to be transfected into a cell.

A green fluorescent protein reporter assay was performed using the pZsGreen1-N1 pDNA (4.7 kbp) as the payload to assess the ability of some polyplexes of the present disclosure to deliver pDNA into the nucleus. In this assay, HEK293T cells were transfected with the aggregated polyplex particles formed with various HQ-X polymers (see Tale 1A) and pZsGreen1-N1 (see Example). The transfection efficiency was evaluated based on the percentage of live cells having green fluorescence; that is, the percentage of live cells producing the green fluorescent protein (GFP), ZsGreen1.

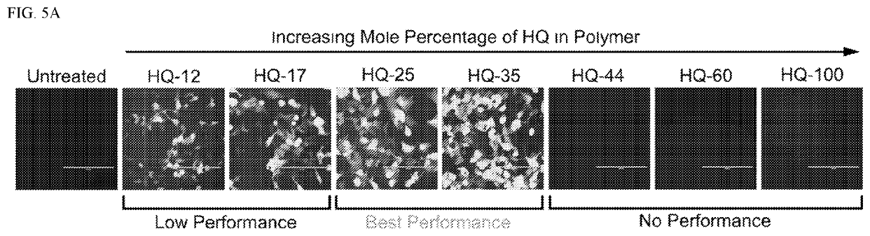

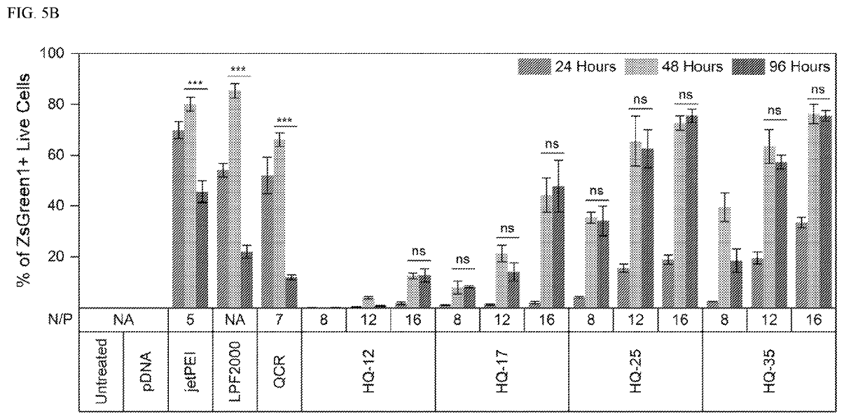

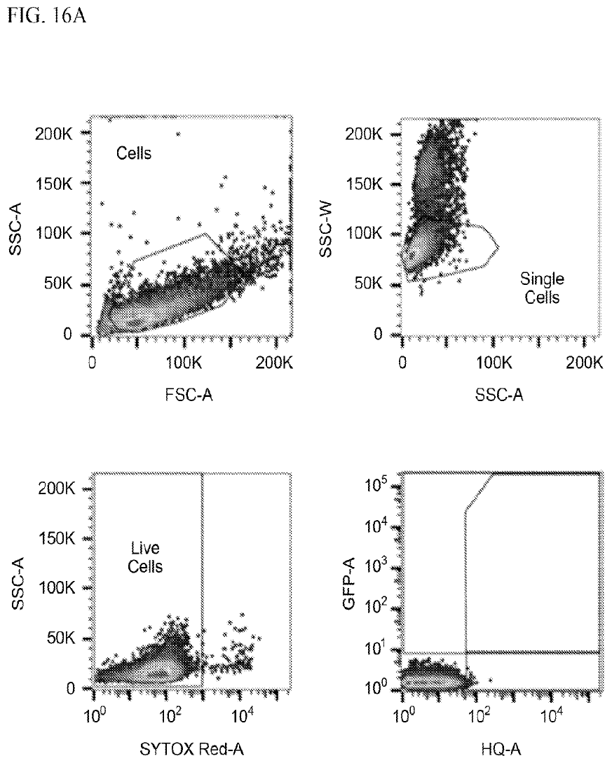

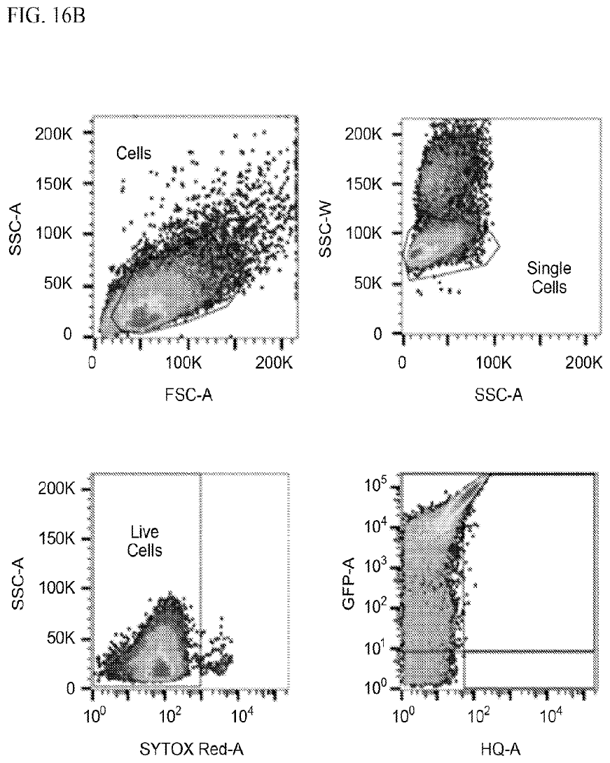

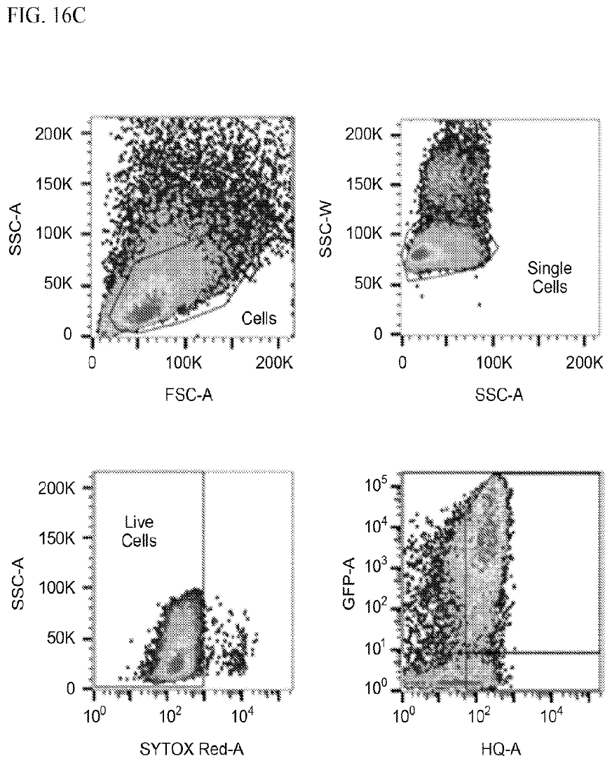

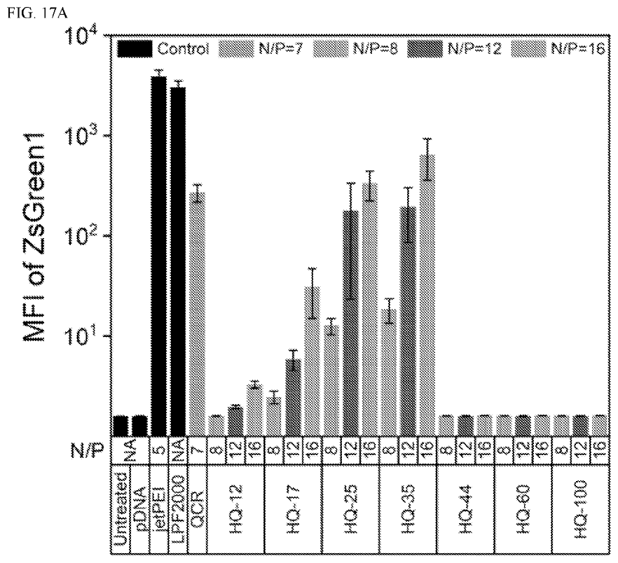

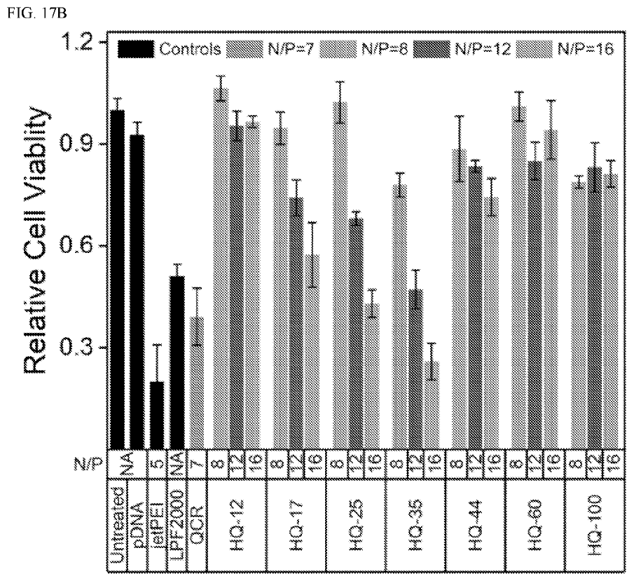

With the increase in mole percentage of HQ in the HQ-X polymers used to form the polyplexes, transfection efficiency increased gradually from HQ-12 to HQ-35 but dropped to zero from HQ-44 onwards to HQ-100. HQ-25 and HQ-35 emerged as the highest-performing polymers from the IIQ-X family with transfection efficiency reaching about 80%, similar in performance to the commercial controls (JETPEI and LIPOFECTAMINE 2000). HQ-44, HQ-60, and HQ-100 were unable to transfect the cells despite having higher n_{Hill} and stronger binding with pDNA than other HQ-X polymers (FIG. 5A, FIG. 16A-16C, an FIG. 17A). The correlation between transfection efficiency and polymer composition was not monotonic (FIGS. 5A and 5B), unlike the trends from dye exclusion assay and potentiometric titration (n_{Hill}) as discussed herein.

Previous studies have proposed high n_{Hill} to be a predictive indicator for high transfection performance, however, this clearly does not apply to the HQ-X polymers tested. For the HQ-X polymers, electrostatic interactions play a minor role and the dominant mode of binding between polymer and pDNA is intercalation which is a non-electrostatic interaction. As a result, the trend in the value of n_{Hill} does not explain the trend in the performance of HQ-X polymers, as n_{Hill} is only relevant for electrostatic interaction. This illustrates that the guiding principles developed for conventional cationic polymers do not necessarily apply to monomers with alternative binding mechanisms such as the polymers disclosed herein.

Compared to cells transfected with polyplexes formed with QCR, cells transfected with polyplexes formed from HQ-12 to HQ-35 polymers have a delayed onset of ZsGreen1 expression. Cells transfected using QCR almost reach a maximum transfection efficiency by 24 hours, however, HQ-12 to HQ-35 containing polyplexes require about 48 hours to reach peak efficiency. This slower response with the HQ-X polymers aligns with the results the analysis of the protein dependency on unpackaging of the polyplexes (FIGS. 4C and 4D). Compared to QCR, polyplexes formed from HQ-X polymers have less affinity to adsorb intracellular proteins and consequently undergo slower unpackaging of the oligonucleotide, ultimately resulting in a delayed onset of ZsGreen1 production. On the other hand, polyplexes formed from QCR undergo rapid protein-mediated unpackaging leading to a faster cellular response in the form of early onset of ZsGreen1 expression.

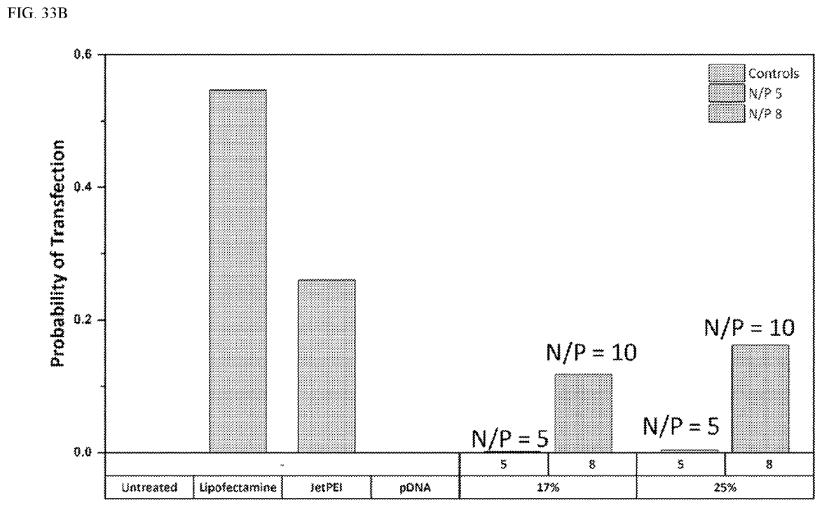

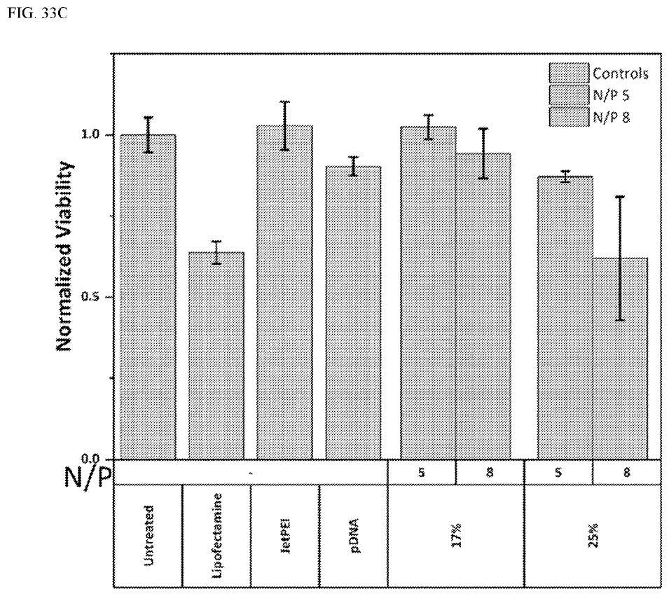

A second green fluorescent protein reporter assay was performed using the pZsGreen1-N1 pDNA (4.7 kbp) as the payload to assess the ability of some polyplexes of the present disclosure to deliver pDNA into the nuclease. In this assay, ARPE-19 cells were transfected with the aggregated polyplex particles formed with HQ-17% and HQ-25% (HQ-X series made via RAFT polymerization). Cells transfected with the polyplex formed with an N/P of 8 gave a higher percentage of cells expressing GFP than those cells transfected with a polyplex formed with an N/P of 5 (FIG. 33A). Additionally, polyplexes formed with an N/P of 8 had a higher probability of transfection than polyplexes formed with an N/P of 5 (FIG. 33B). Probability of transfection was calculated by multiplying the percentage of GFP expressing cells (normalized between 0 to 1) with the normalized viability. Cells transfected with polyplexes formed with an N/P of 8 showed a slightly lower cell viability than cells transfected with polyplexes formed with an N/P of 5. Cells transfected with the polyplex formed with a polymer that had 25 mol-% HQ gave a slightly higher percentage of cells expressing GFP than those cells transfected with a polyplex with a polymer that had 17 mol-% HQ (FIG. 33A). Additionally, polyplexes formed with a polymer that had 25 mol-% HQ had a higher probability of transfection than polyplexes formed with a polymer that had 17 mol-% HQ (FIG. 33B). Cell transfected with polyplexes formed with a polymer that had 25 mol-% HQ showed less cell viability than cells transfected with polyplexes formed with a polymer that had 17 mol-% HQ (FIG. 33C).

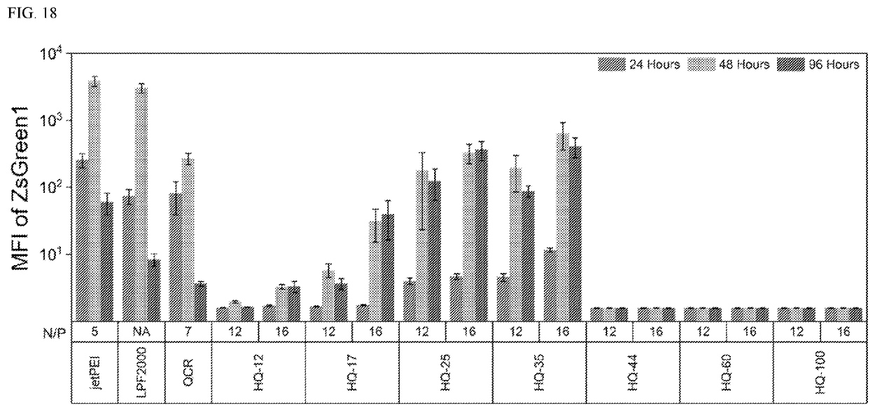

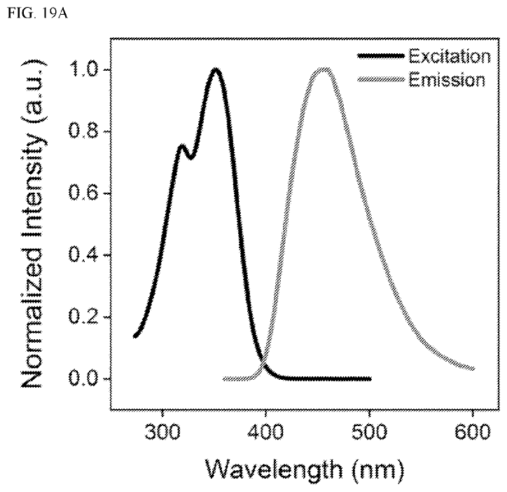

For therapeutic applications, a sustained effect of transfection (e.g., gene expression) can be useful for reducing the frequency of dose administration. To evaluate the ability of polyplexes formed with HQ-X polymers in sustaining gene expression over a longer period of time, transfection efficiency was quantified 96 hours after transfection, which is 48 hours longer than the typical standard in this field (FIG. 5B, FIG. 18). For polyplexes formed with QCR and the commercial controls, the ZsGreen1+ population peaked at 48 hours but diminished significantly by 96 hours. However, for the cells transfected using polyplexes formed from HQ-17 to HQ-35 polymers, the ZsGreen1+ population remained high from 48 hours to 96 hours, outperforming QCR as well as the commercial controls (FIG. 5B). The resistance to protein-mediated unpackaging of the polyplexes and consequent slow release of the pDNA payload may be responsible for the sustained transfection efficiency observed for polyplexes formed from HQ-17 to HQ-35 polymers. For polyplexes formed from HQ-44, HQ-60, and HQ-100 polymers, fluorescent cells were not observed even 96 hours after transfection suggesting that for polymers with high HQ content, pDNA release does not occur even after 96 hours due to very tight polymer-pDNA association. Overall, these results collectively indicate that the HQ-X class of polymers have more stable pDNA binding characteristics that could benefit packaging, storage, and sustained in vivo delivery for nucleic acid payload.

It is thought that cells can internalize particles as large as 5 micrometers in diameter but generally the extent of cellular uptake decreases rapidly with an increase in particle size, specifically for particles that are larger than 1 micrometer in diameter. As such, it was expected that the larger size and the flocculant nature of the aggregates formed from the dilution of polyplexes formed from HQ-44, HQ-60, and HQ-100 (FIG. 11A-11I and FIG. 12) were going to be an obstacle for cellular internalization that ultimately rendered them incompatible for transfection.

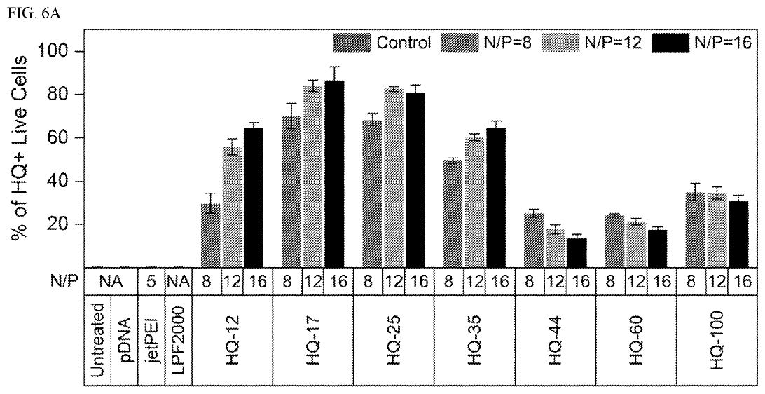

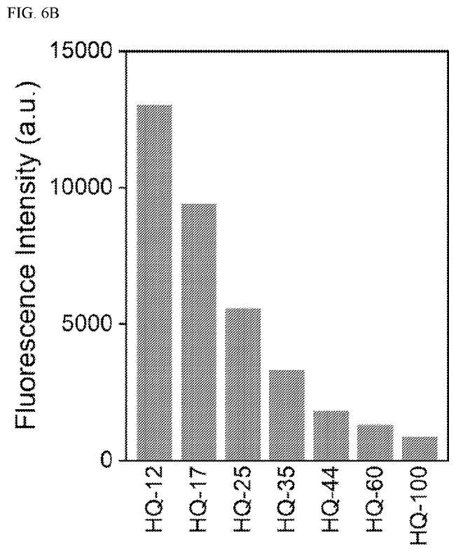

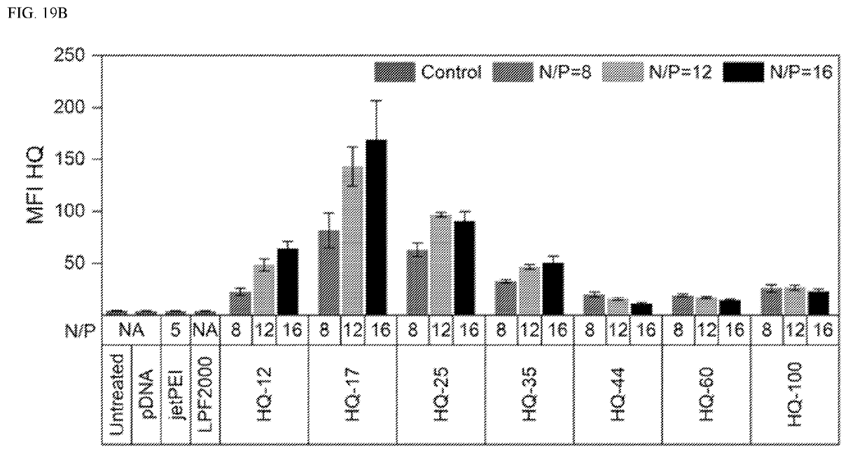

The extent of polyplex internalization (percentage of live cells containing polyplexes) was done using the inherent fluorescence of the HQ and HQ-X polymers (λₑₓ=352 nm, λₑₘ=460 nm) (FIG. 19A). Cells were analyzed 24 hours after transfection using flow cytometry by excitation with a UV laser (350 nm) and observing cells for blue fluorescence. Polyplexes were present in cells transfected using HQ-12, HQ-17, HQ-25, and HQ-35. Polyplexes were also present in cells transfected using HQ-44, HQ-60, and HQ-100, although to a relatively lesser extent compared to the HQ-12, HQ-17, HQ-25, and HQ-35 (FIG. 6A and FIG. 19B). The fluorescence of HQ is advantageous in determining the cellular uptake of polyplexes, but care must be taken when interpreting the results because the fluorescence intensity of HQ-X polymers decreases with an increase in the mole percentage of HQ due to self-quenching (FIG. 6B).

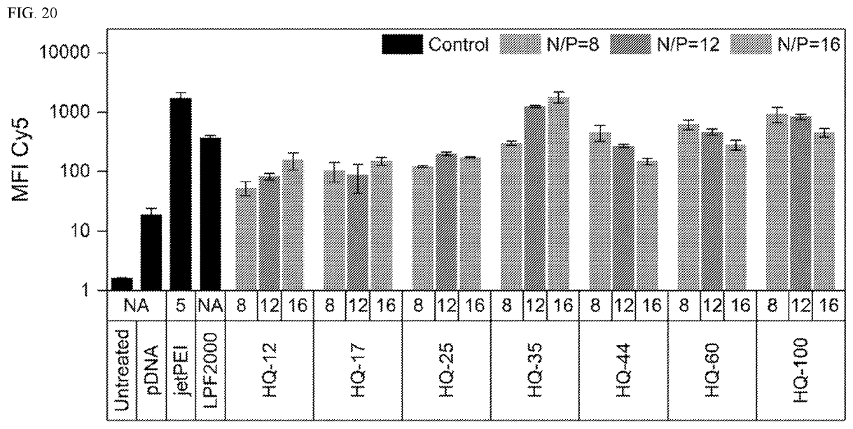

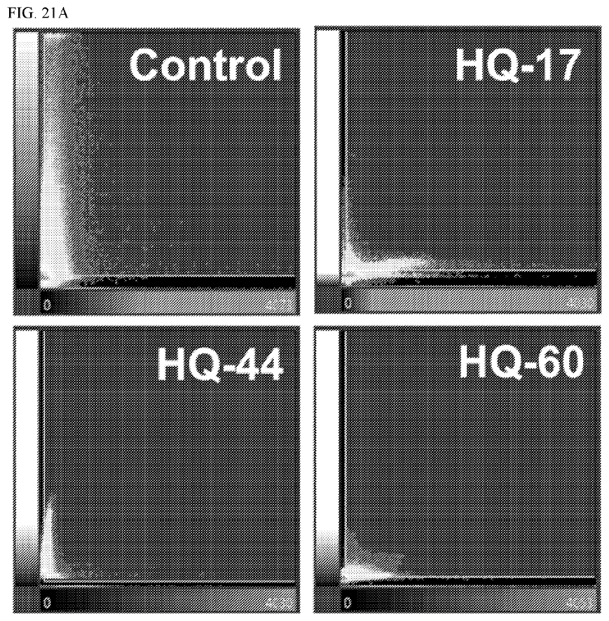

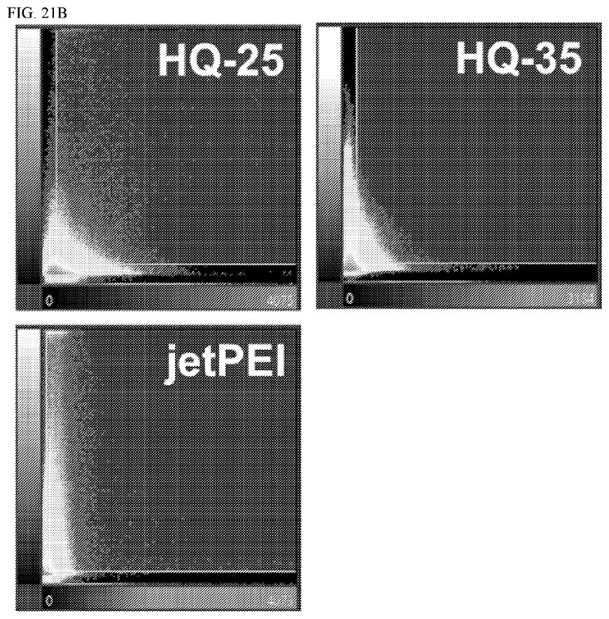

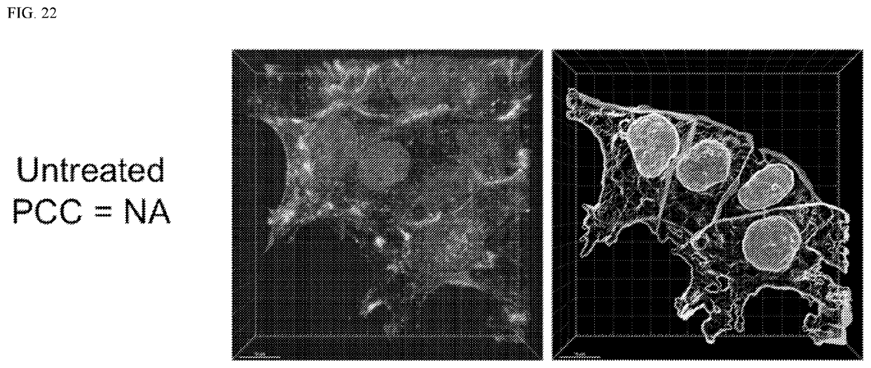

The extent of polyplex internalization was further probed by transfecting cells using polyplexes that included the HQ-X polymers and a Cy5-labeled pDNA. Twenty-four hours after transfection, cells were analyzed for Cy5 fluorescence (λex=651 nm, λem=670 nm), using flow cytometry (FIG. 7 and FIG. 20; see Example). Cy5 is a more sensitive fluorophore than HQ, therefore, examining cellular uptake of polyplexes based on Cy5 intensity was expected to have lower occurrences of false negative events than in the case of HQ fluorescence. The results indicated that all polyplexes formed from all the tested polymers were able to deliver the Cy5-labeled pDNA inside the cells with half of the formulations achieving more than 80% internalization efficiency (% of Cy5+ live cells), even including most formulations of polyplexes made with HQ-44, HQ-60, HQ-100. Comparing the cellular uptake results from this experiment with the data in FIG. 6A, where HQ fluorescence was used, it was observed that for polyplexes made from HQ-12, HQ-17, and HQ-25 polymers, the percentage of Cy5+ cells matches closely with the percentage of HQ+ cells. However, for polymers with a higher mole percentage of HQ (≥35%), the use of HQ fluorescence leads up to 65% underestimation of cellular uptake due to significant fluorescence quenching of HQ (FIG. 6A and FIG. 7). These results indicate that at low molar incorporation of HQ (≤25%), the inherent fluorescence of these polymers can be used to directly quantify the extent of cellular uptake of the polyplexes, without requiring additional fluorescent labeling. Additionally, the lower transfection efficiency of polyplexes having higher molar amounts of HQ in the polymers (e.g., HQ-44, HQ-60, and HQ-100) is not due to low cellular internalization as a result of their size or flocculant nature.

Based on the combined results of dye exclusion assays, polyplex flow cytometry, cellular uptake, and transfection experiments, an obstacle for successful transfection and subsequent gene expression using polyplexes formed with polymer HQ-44, HQ-60, and HQ-100 is the delayed release of pDNA from the polyplexes after internalization into the cytoplasm. For successful transfection and transgene expression, the pDNA needs to be released from the polyplexes inside the cell and get translocated into the nucleus. Polymers with a high mole percentage of HQ (≥44%) likely bind too strongly with pDNA not allowing it to be released from the polyplexes inside the cytoplasm, preventing its translocation to the nucleus, and production of ZsGreen1. Though QCR binds to pDNA as tightly as HQ-44, HQ-60, and HQ-100, a higher affinity for proteins allows the pDNA to be released from polyplexes of QCR making it functional. On the other hand, at lower percentages of HQ (≤17%), the binding between pDNA and polymer is not sufficient to form stable polyplexes resulting again in poor transfection efficiency. Based on the transfection results, polyplexes formed with polymers having 25 mol-% to 35 mol-% HQ allow for polymer-pDNA binding and post-internalization pDNA release.

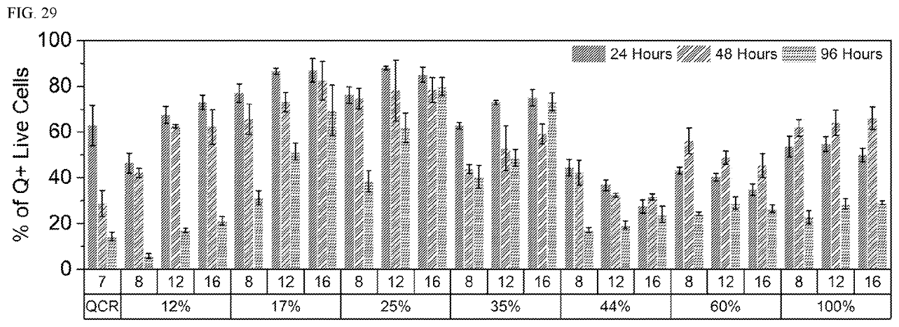

FIG. 29 shows the fate of the polyplexes that formed from the HQ-X series of polymers internalized by the cells. The intrinsic fluorescence of quinoline was used as a proxy for the HQ monomers of the polymers of the polyplexes (determine the Q+ signal). For the majority of the formulations, the percent of live cells with a quinoline signal decreases over time. The decrease may be due two reasons. First, the polyplexes and/or the polymers are ejected out of the cells. Second, in some instances, cell division may lead to an uneven split of polyplexes and/or polymers in daughter cells since they are large particles and do not evenly distribute across the cells. As such, some cells may not have received polyplexes and/or polymers during cell division. For the polyplexes made using the HQ-X polymers having 44 mol-%, 60 mol-%, or 100 mol-% HQ incorporation, the initial polyplex particles are very large for internalization but protein in the media may break down the larger aggregates into smaller polyplex particles that are easier to internalize. This may explain the increase in Q+ cells from 24 hours to 48 hours.

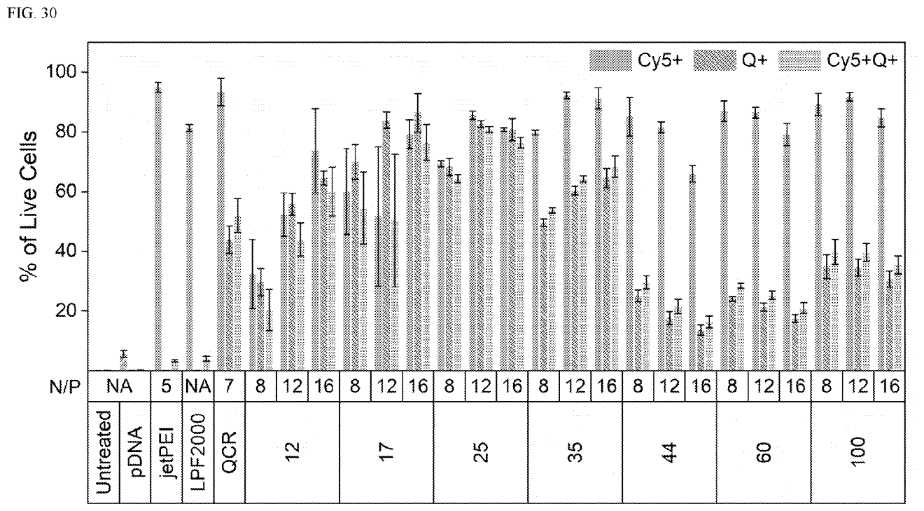

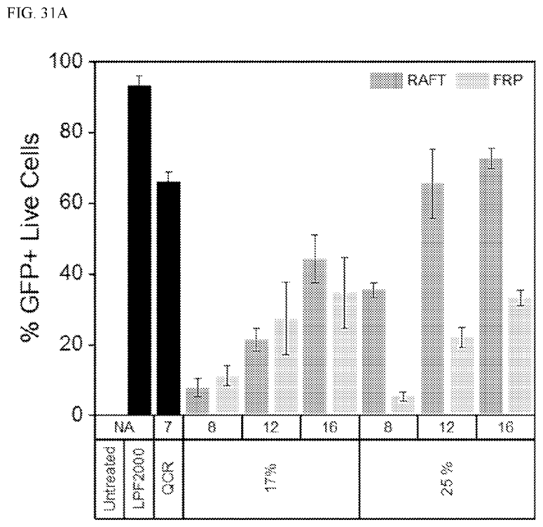

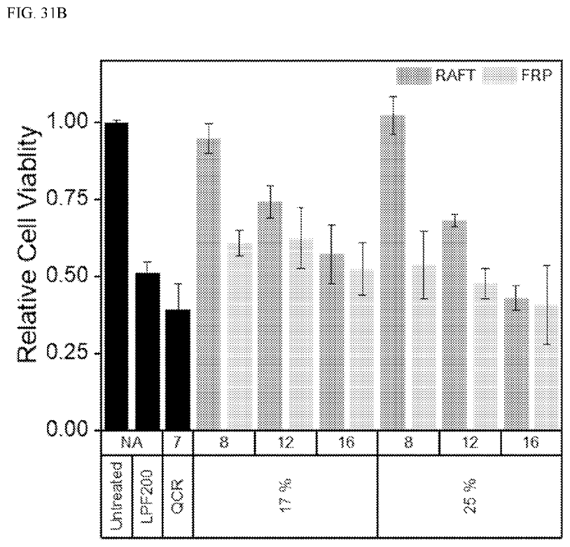

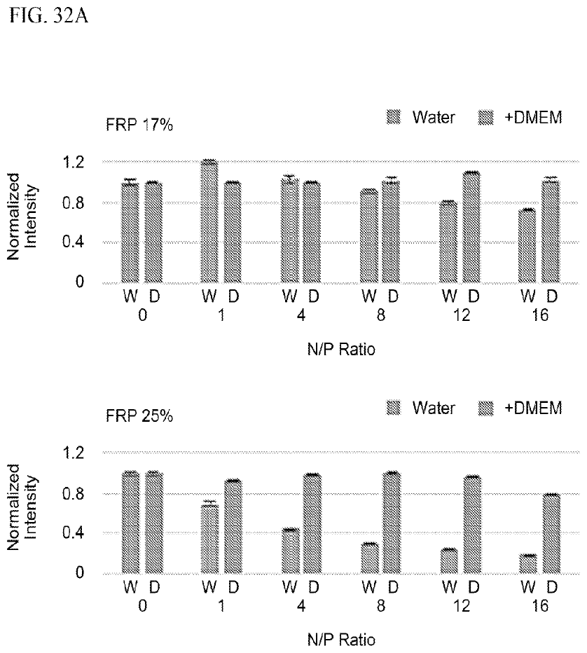

FIG. 30 compares the internalization of Cy5 (Cy5+) and quinoline (Q+; proxy for the HQ monomers of the polymers of the polyplexes, determined using the intrinsic fluorescence of quinoline) at 24 hours post transfection. The polyplexes made using polymers that had a lower mol-% of HQ (12 mol-% and 17 mol-%) had more Q+ cells than Cy5+ cells. This implies that many of the cells had internalized aggregates/particles that did not include the Cy5-labelled pDNA, possibly as a result of weaking binding between the polymer and pDNA. The polyplexes made using polymers that had a higher mol-% of HQ (35 mol-%, 44 mol-%, 60 mol-%, and 100 mol-%) had more Cy5+ cells than Q+ cells. This may be the result of a self-quenching effect of the polymer. With increasing the amount HQ in the polymer chain, the self-quenching intensifies leading to a less accurate detection of Q+ live cells.

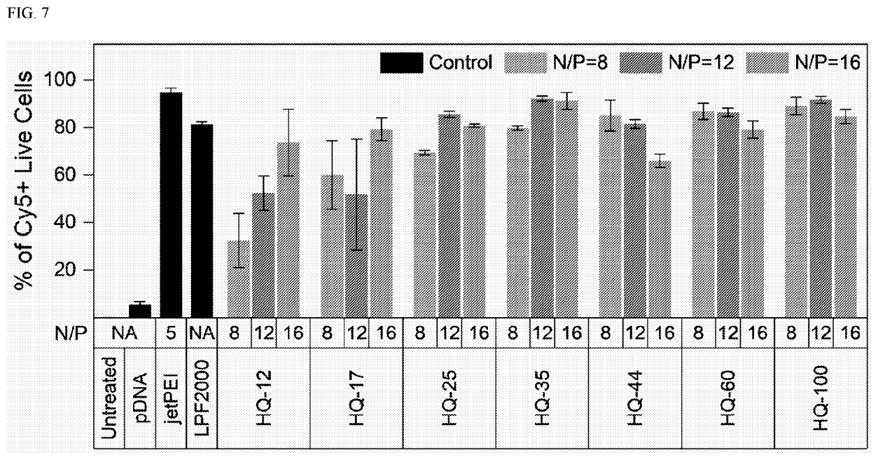

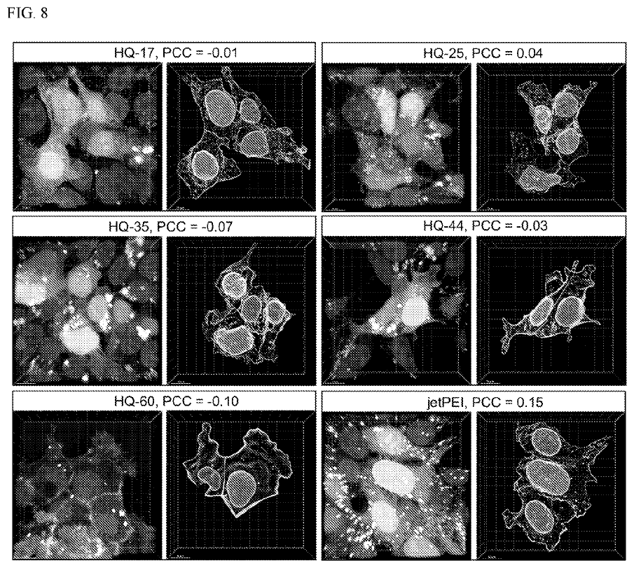

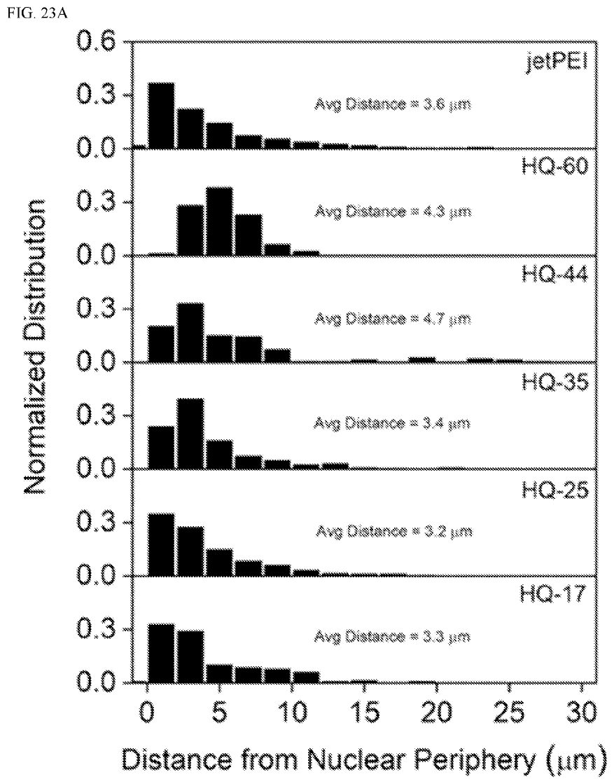

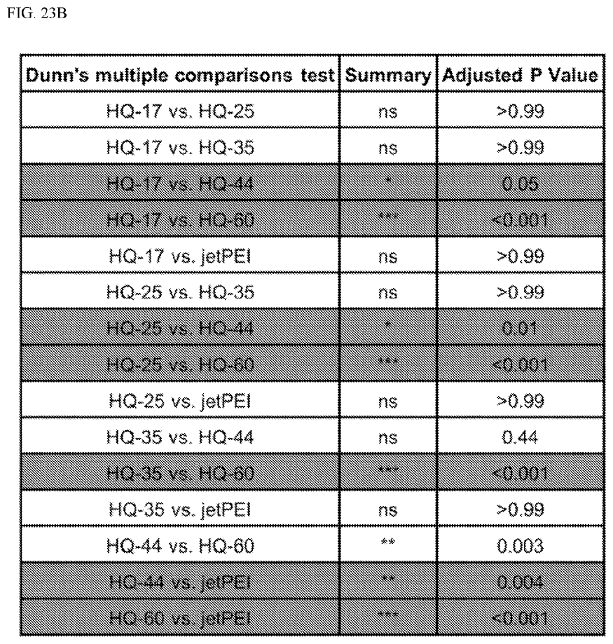

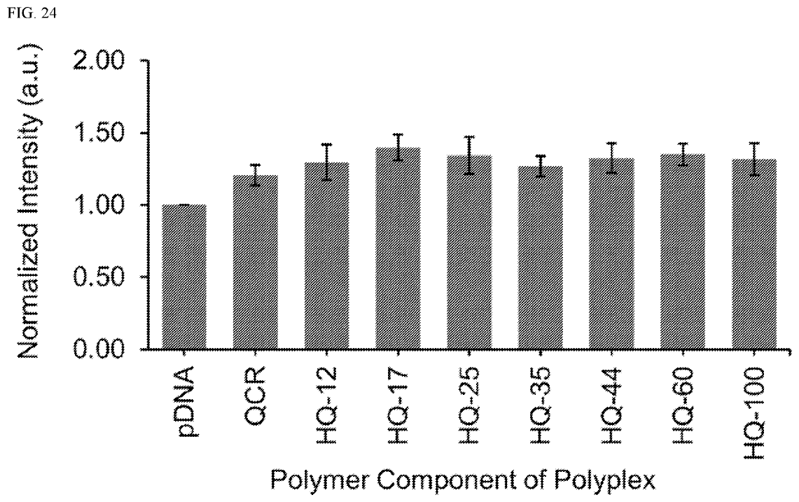

Colocalization experiments using confocal laser scanning microscopy on fluorescently labeled HEK293T cells were performed to probe the subcellular localization and intracellular behavior of polyplexes. The cells were transfected with polyplexes made from five out of the seven polymers from the HQ-X family (HQ-17, HQ-25, HQ-35, HQ-44, HQ-60) using Cy5-labeled pZsGreen1-N1 pDNA as the payload. Twenty-four hours after transfection, the cells were fixed, and the lysosomal associated membrane protein-2 (LAMP-2) were stained with a primary antibody and an ALEXAFLUOR 555 labeled secondary antibody. The colocalization of pDNA (Cy5) and lysosome (ALEXAFLUOR 555) was evaluated in the form of Pearson's correlation coefficient (PCC) (FIG. 8 and FIGS. 21A-21B). For all five polymers, the PCC score was found to be close to zero (between-0.1 and 0.1) implying no correlation in the localization of the pDNA payload and the lysosomes. This suggests that all five of the HQ-X polymers that were tested were able to facilitate endosomal escape of the pDNA payload from endosomal compartments. Due to having low pKa (Table 1A), the large reserves of unprotonated amines in the HQ-X polymers likely act like a proton sponge and facilitate endosomal escape. As a comparison, the PCC score from the JETPEI treated cells was higher (0.15) than the cells transfected using HQ-X, which further highlights the ability of HQ-X polymers for endosomal escape (FIG. 22). Furthermore, the low PCC scores for HQ-44 and HQ-60, further indicate that poor release of pDNA from the polyplexes is the primary bottleneck against productive transfection for polymers with high HQ content (≥44%). The distribution of the distances between the internalized Cy5-labeled pDNA and the nuclear membrane were compared. HQ-17, HQ-25, and HQ-35 were able to shuttle pDNA 20% to 30% closer to the nuclear periphery than IIQ-44 and IIQ-60 (FIGS. 23A and 23B).

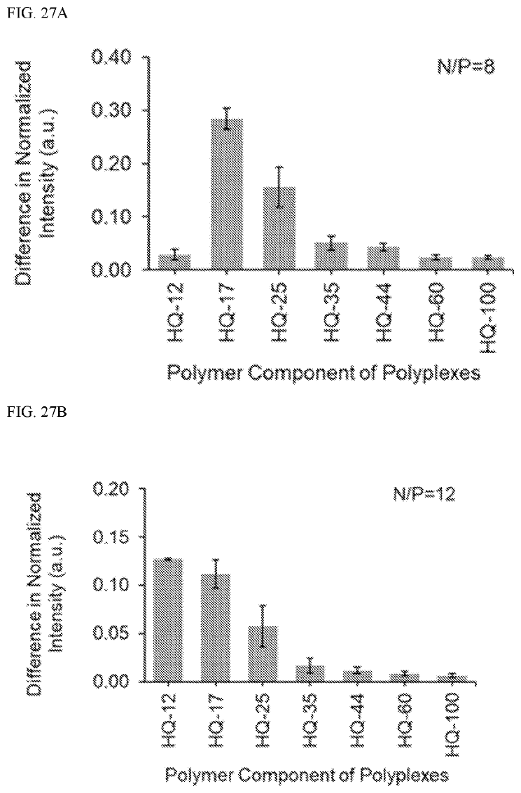

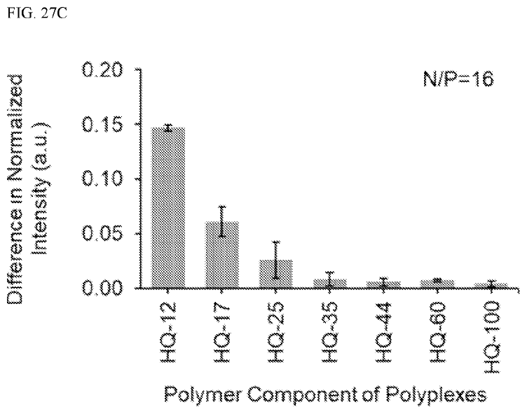

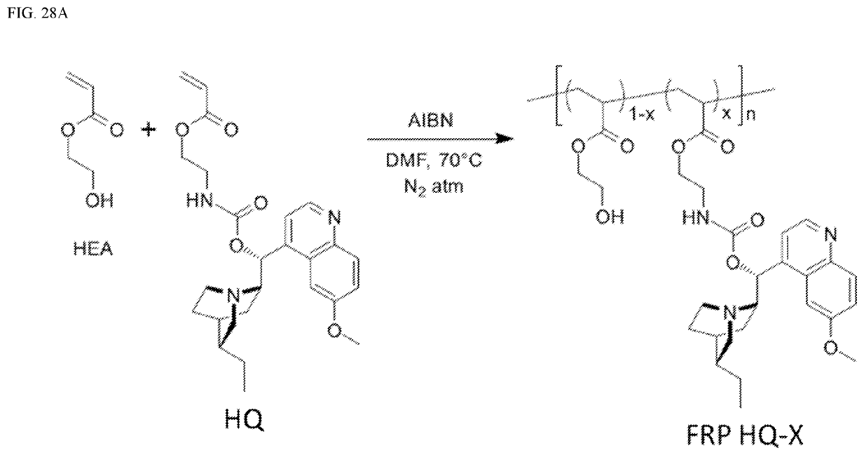

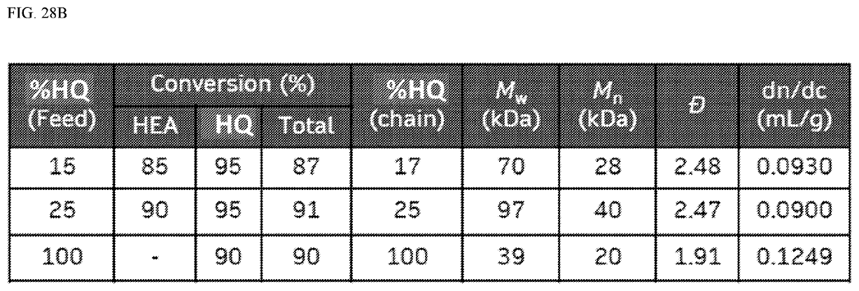

The poor release of pDNA from HQ-44, HQ-60, and HQ-100 was further probed by performing a dye exclusion assay in the presence of heparin. Heparin is a polyanion known to compete with pDNA to bind with cationic polymers (FIGS. 27A-27C). The extent of pDNA release from polyplexes, caused by heparin, decreases with the increase in HQ content of the polymer indicating that among the HQ-X polymers, HQ-44, HQ-60, and HQ-100 not only bind with pDNA the strongest but also have the least amount of pDNA release

In another aspect, the present disclosure describes a transfection kit. The kit includes a transfection reagent of the present disclosure. The transfection reagent may be provided in a solution. In one or more embodiments, the transfection reagent may be provided in an acidic solution having a pH of 1 to 5. In one or more embodiments, the kit may include a serum free medium. In one or more embodiments, the kit may include a serum-containing medium. In one or more embodiments, the kit may include ultrapure water. In one or more embodiments, the kit may include instructions.

In another aspect, the present disclosure describes synthetic ancillary agents that include a quinoline containing monomer and/or polymer that includes a quinoline containing monomer.

The quinoline containing monomer and/or polymer that includes a quinoline containing monomer may be any quinoline containing monomer and/or polymer as described elsewhere herein. The synthetic ancillary agents may be used as synthetic chiral ancillary agents. In embodiment, synthetic chiral ancillary agents may allow for the formation of a desired stereoproduct. In embodiments, synthetic chiral ancillary agents may allow for the increased yield of a desired stereoproduct. In embodiments, the synthetic chiral ancillary agent may be a catalyst.

### Definitions

As used herein, the following terms shall have the indicated meanings.

As used herein, “alkyl” or “alkyl group” refers to a fully saturated, straight, or branched hydrocarbon chain having from one to twelve carbon atoms, and which is attached to the rest of the molecule by a single bond.

As used herein, “alkene” refers to carbon-carbon double bond. An alkene is a functional group that may be a part of a larger molecule. An alkene may be a polymerizable functional group.

When a group is present more than once in a formula described herein, each group is independently selected, whether specifically stated or not. For example, when more than one Y group is present in a formula, each Y group is independently selected. Furthermore, subgroups contained within these groups are also independently selected. For example, when each Y group contains an R¹, each R¹ is also independently selected.

As used herein, the symbols

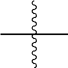

and “-” (hereinafter can be referred to as “a point of attachment bond or point of attachment”) when used in the context of a compound, denote a bond that is a point of attachment between two chemical entities, one of which is depicted as being attached to the point of attachment bond and the other of which is not depicted as being and attached to the point of attachment bond. For example,

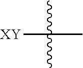

and
—XY” indicate that the chemical entity “XY” is bonded to another chemical entity via the point of attachment bond.

The point of attachment of the chemical group to the compound may be described in several ways. For example, in some embodiments, the organic group may be described as the monovalent or radical of the respective functional group (e.g., alkyl for alkane, alkenyl for an alkene containing group). In some embodiments, where a general formula is shown with a covalent bond connecting the chemical group to a compound, the chemical group may be described as the common functional group that is or is contained within the chemical group. For example, if the chemical group R is described relative to the formula CH₃CH₂CH₂—R, the chemical group may be described, for example, as an alkene.

The terms “alkenyl” and “alkenyl group” refers to a univalent group that is a radical of an alkene and includes groups that are linear, branched, cyclic, or combinations thereof. An alkenyl group has one or more double bonds. The location of the double bond may be anywhere along the alkenyl. The radical may be a part of the double bond (e.g., ⋅CHCH—R). The radical may be a part of a single bond (e.g., ⋅CH₂—R).

As used herein, “phenyl ether” refers to a compound of the formula R—O—R′ where R is a phenyl group and R′ is alkyl. R′ may be methyl, ethyl, n-propyl, iso-propyl, or butyl.

As used herein, the terms “formed from” and “polymerized from” are open ended and may include other components that may not be expressly described relative to the subject that is formed from or polymerized from the stated components. For example, a polymer formed from or polymerized from a quinoline-containing monomer may include capping groups or other groups not expressly mentioned.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments” “in one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

The invention is defined in the claims. However, below there is provided a non-exhaustive listing of non-limiting exemplary embodiments. Any one or more of the features of these embodiments may be combined with any one or more features of another example, embodiment described herein.

### EXEMPLARY EMBODIMENTS

Embodiment 1. Embodiment 1 is quinoline-containing compound of the general formula IX:

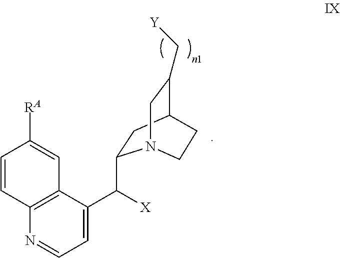

In the general formula IX, R^{A} is H or OCH₃. n1 is an integer from 1 to 5. X is OH, H, or of the general formula XII, XIII, or XIV:

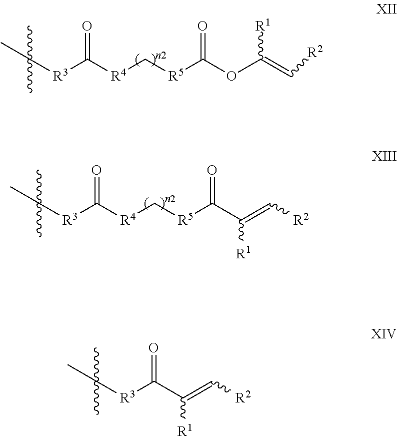

In formulas XII, XIII, and XIV, R³, R⁴, and R⁵, are each independently O, NH, NR²⁰, or S; R²⁰ is methyl, ethyl, or propyl; R¹ and R² are each independently H or alkyl; and n2 is an integer from 1 to 10. In the general formula IX, Y may be an alkyl (e.g., CH₃ and CH₂CH₃) an alkenyl (e.g., CHCH₂), or of general formula XV, XVI, XVII, or XIX.

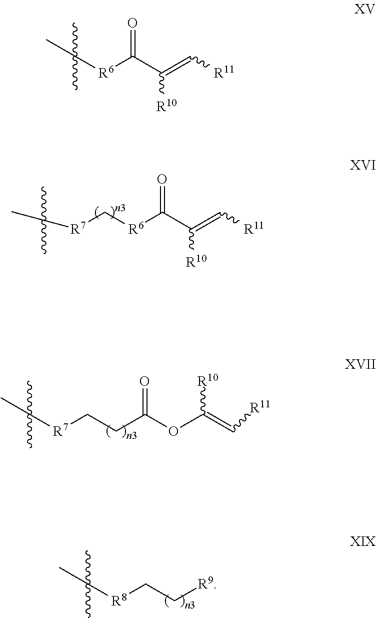

In formulas XV, XVI, XVII, and XIX, R⁶, R⁷, R⁸ are each independently O, NH, NR²¹, or S; R²¹ is alkyl; R¹⁰ and R¹¹ are each independently II or methyl; R⁹ is NR²²R²³, SH, NH₂, NH₃, or OH where R²² and R²³ are each independently H, methyl, ethyl, n-propyl, or isopropyl; and n3 is zero or an integer from 1 to 5.
Embodiment 2. Embodiment 2 is the compound of embodiment 1 where X is of the general formula XII, XIII, or XIV and Y is CHCH₂, CH₂CH₃, CH₃.
Embodiment 3. Embodiment 3 is the compound of Embodiment 1 or Embodiment 2, wherein R^{A} is OCH₃.
Embodiment 4. Embodiment 4 is the compound of any one of Embodiments 1 through 3, where Y is CH₃.
Embodiment 5. Embodiment 5 is the compound of Embodiment 1, wherein the compound

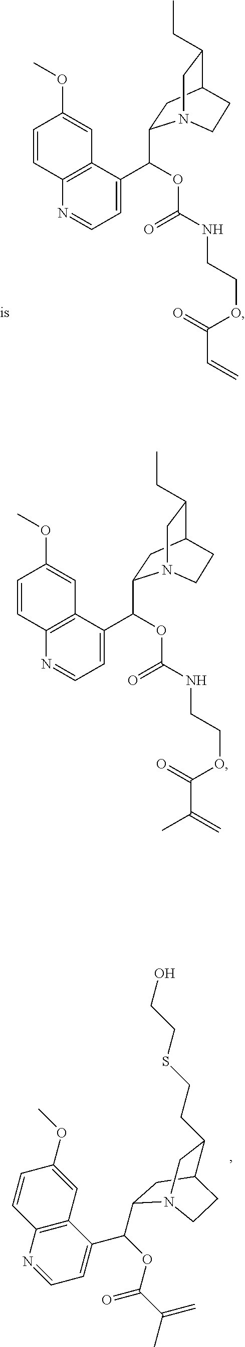 [SMILES: C=CC(=O)OCCNC(=O)OC(c1ccnc2ccc(OC)cc12)C1CC2CCN1CC2CC ; C=C(C)C(=O)OCCNC(=O)OC(c1ccnc2ccc(OC)cc12)C1CC2CCN1CC2CC ; C=C(C)C(=O)OC(c1ccnc2ccc(OC)cc12)C1CC2CCN1CC2CCSCCO]

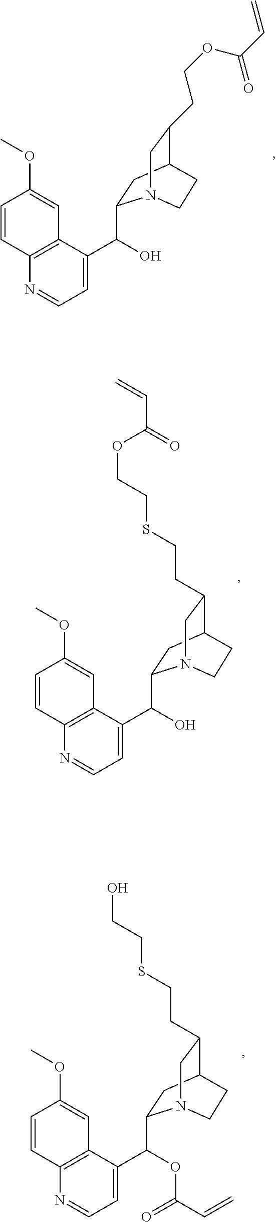 [SMILES: C=CC(=O)OCCC1CN2CCC1CC2C(O)c1ccnc2ccc(OC)cc12 ; C=CC(=O)OCCSCCC1CN2CCC1CC2C(O)c1ccnc2ccc(OC)cc12 ; C=CC(=O)OC(c1ccnc2ccc(OC)cc12)C1CC2CCN1CC2CCSCCO]

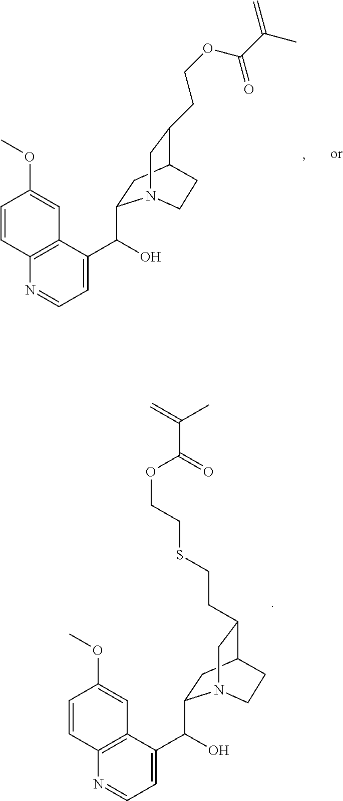 [SMILES: C=C(C)C(=O)OCCC1CN2CCC1CC2C(O)c1ccnc2ccc(OC)cc12 ; C=C(C)C(=O)OCCSCCC1CN2CCC1CC2C(O)c1ccnc2ccc(OC)cc12]

Embodiment 6. Embodiment 6 is the compound of Embodiment 1, where the compound is

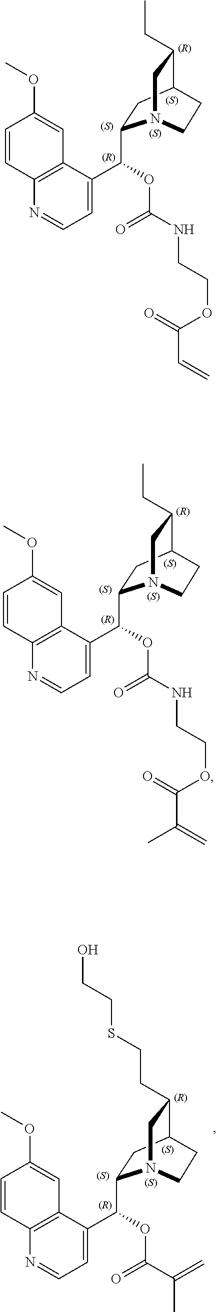

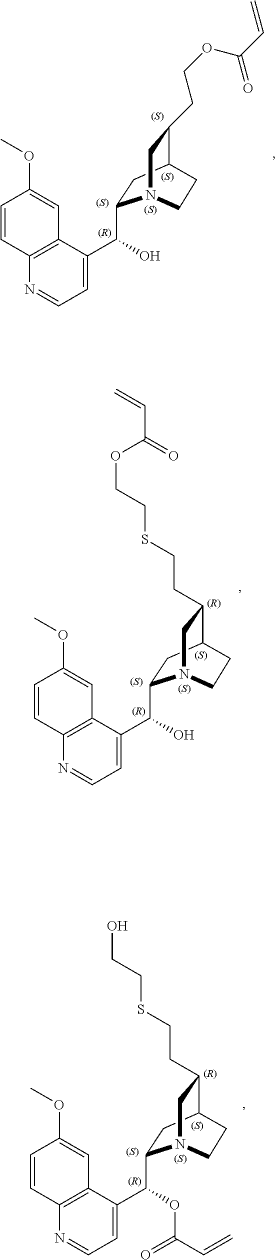

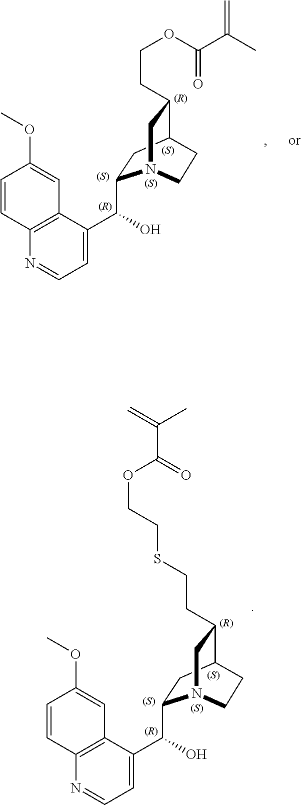

Embodiment 7. Embodiment 7 is a polymer that includes; that is, polymerized a quinoline-containing compound (i.e., quinoline-containing monomer) monomer of any one of Embodiments 1 through 6.
Embodiment 8. Embodiment 8 is a polymer of Embodiment 7, where the polymer is a homopolymer polymerized from the quinoline-containing monomer.
Embodiment 9. Embodiment 9 is a polymer of Embodiment 7, where the polymer is copolymer polymerized from the quinoline-containing monomer and a second monomer.
Embodiment 10. Embodiment 10 is a polymer of Embodiment 9, where the copolymer includes 15 mol-% or greater the quinoline-containing monomer.
Embodiment 11. Embodiment 11 is a polymer of Embodiment 9 or Embodiment 10, where the copolymer includes 15 mol-% to 35 mol-% of the quinoline-containing monomer.
Embodiment 12. Embodiment 12 is a polymer of any one of Embodiments 9 through 11, where the copolymer includes 20 mol-% to 30 mol-% of the quinoline-containing monomer.
Embodiment 13. Embodiment 13 is a polymer of any one of Embodiments 9 through 12, where the second monomer includes an acrylate monomer, a methacrylate monomer, an acrylamide monomer, a methacrylate monomer, or a vinyl monomer.
Embodiment 14. Embodiment 14 is a polymer of Embodiments 13, where the acrylamide monomer is acrylamide; (2-hydroxyethyl) acrylamide; N-isopropyl acrylamide; or N,N-dimethylacrylamide (DMA).
Embodiment 15. Embodiment 15 is a polymer of Embodiments 13, where the acrylate monomer is (2-hydroxyethyl) acrylate or methyl acrylate.
Embodiment 16. Embodiment 16 is composition (e.g., a transfection composition) comprising a polyplex transfection complex. The polyplex transfection complex includes the polymer of any one of Embodiments 7 through 15 and an oligonucleotide. The composition also includes a transfection medium.
Embodiment 17. Embodiment 17 is the composition of Embodiment 16, wherein the oligonucleotide includes a plasmid DNA.
Embodiment 18. Embodiment 18 is the composition of Embodiment 16 or Embodiment 18, wherein the oligonucleotide comprises a plurality of phosphate groups, the quinoline-containing monomer comprises an amine; and a ratio of moles of the amine to moles of the plurality of phosphate groups is 2 to 20 or 10 to 16.
Embodiment 19. Embodiment 19 is a method of forming the polyplex transfection complex of any one of Embodiments 16 through 18. The method includes mixing the polymer with the oligonucleotide to create a first mixture. The method further includes incubating the first mixture for a period of time to form a second mixture comprising the polyplex transfection complex.
Embodiment 20. Embodiment 20 is the method of Embodiment 19, where the first mixture is at a pH of 1 to 5.
Embodiment 21. Embodiment 20 is the method of Embodiment 19 or Embodiment 20, where the first period of time is 5 minutes to 40 minutes.
Embodiment 22. Embodiment 22 is the method of any one of Embodiments 19 through 21, where the method further includes diluting the second mixture with a transfection medium to form a third mixture comprising the polyplex transfection complex.
Embodiment 23. Embodiment 23 is the method of Embodiment 22, where the transfection medium is serum free.
Embodiment 24. Embodiment 23 is the method of any one of Embodiments 19 through 23, the method further including contacting a cell with the third mixture to form a transfection mixture; incubating the transfection mixture for a transfection time; and quenching the transfection mixture.
Embodiment 25. Embodiment 25 is the method of Embodiment 25, where the third mixture has a pH of 5 to 8.
Embodiment 26. Embodiment 26 is the method of Embodiment 24 or 25, where the transfection time is 20 minutes to 40 minutes.
Embodiment 27. Embodiment 27 is the method of any one of Embodiments 24 through 26, where quenching the transfection mixture includes adding a serum-containing media to the transfection mixture
Embodiment 28. Embodiment 28 is a kit that includes a transfection agent that includes the polymer of any one of Embodiments 7 through 15.
The present invention is illustrated by the following example. It is to be understood that the particular examples, materials, amounts, and procedures are to be interpreted broadly in accordance with the scope and spirit of the invention as set forth herein.

### EXAMPLES

### Materials:

Synthesis of Polymers: Quinine (anhydrous, 99% total base with ≤5% dihydroquinine) was purchased from Alfa Aesar (Tewksbury, MA). Hydroquinine (98%), 2-hydroxyethyl acrylate (HEA); 4-cyano-4-[(dodecylsulfanylthiocarbonyl)sulfanyl]pentanoic acid (CDP); 2-(dodecylthiocarbonothioylthio)-2-methylpropionic acid (DDMAT); azobisisobutyronitrile (AIBN); 4,4′-azobis(cyanovaleric acid) (V-501); silica gel (technical grade, pore size 60 Å, 70-230 mesh); and heparin ammonium salt from porcine intestinal mucosa were purchased from Sigma-Aldrich (St. Louis, MO). 2-isocyanatoethyl acrylate (stabilized with BHT) was purchased from TCI Chemicals (Portland, Oregon). SPECTRA/ROR pre-wetted RC dialysis tubing (MW cutoff~ 1 kDa) was purchased from Spectrum Chemical Mfg. Corp (New Brunswick, NJ). The tubing was soaked in and rinsed with Milli-Q water prior to use.

Transfection: The pZsGreen1-N1 plasmid DNA (4.7 kbp) was purchased from Aldevron (Fargo, ND). CCK-8 cell counting kit was purchased from Bimake (Houston, TX). LIPOFECTAMINE 2000, SYTOX™ red dead cell stain, PICOGREEN (QUANT-IT PICOGREEN, dsDNA reagent), were purchased from ThermoFisher Scientific (Waltham, MA). CELLSCRUB buffer was purchased from Genlantis (San Diego, CA). LABEL IT Nucleic Acid Labeling Kit, Cy5 was purchased from Mirus Bio (Madison, WI). JETPEI was purchased from Polyplus-transfection (New York, NY).

Cell Culture: Dulbecco's Modified Eagle Medium (DMEM; high glucose, pyruvate, and GlutaMAX supplemented); FLUOROBRITE DMEM; Trypsin-EDTA (0.05%) with and without phenol red; Phosphate Buffered Saline (PBS) pH=7.4; UltraPure DNAse/RNAse-Free distilled water (DI H₂O); antibiotic-antimycotic (100x); and the flow cytometry sub-micron particle size reference kit (F13839) were purchased from Life Technologies ThermoFisher Scientific (Carlsbad, CA). Heat inactivated fetal bovine serum (HI FBS) was purchased from Corning Life Sciences (Durham, NC). Cell line of human embryonic kidney cells (HEK293T) were engineered from the laboratory of Mark Osborne at the University of Minnesota with a traffic light reporter system. Subclones were made at the Genome Engineering Shared Resource (Minneapolis, MN) to obtain a stable cell line.

Microscopy: For confocal microscopy imaging, cells were plated in 8-well chambered slides purchased from IBIDI (Gräfelfing, Germany). Anti-LAMP2 antibody [H4B4]-lysosome marker and normal goat serum was purchased from abcam (Waltham, MA). Goat anti-Mouse IgG (H+L) highly cross-adsorbed secondary antibody, ALEXAFLUOR 555 and SLOWFADE glass (with DAPI) soft-set antifade mountant was purchased from Invitrogen (ThermoFisher Scientific; Waltham, MA). Gelatin from porcine skin (gel strength~300 g Bloom) and bovine serum albumin was purchased from Sigma Aldrich (St. Louis, MO). 16% paraformaldehyde aqueous solution was purchased from Electron Microscopy Sciences (Hatfield, PA). TRITON X-100 was purchased from Integra (Renton, WA). Steriflip vacuum-driven filtration system (0.22 micrometers) was purchased from Millipore (Burlington, MA).

Instrument Details: NMR spectra were recorded on AX-400 Bruker Avance III IID (Billerica, MA). Mass spectra were recorded on BioTOF II ESI-TOF Mass Spectrometer. Size exclusion chromatography was performed on Agilent INFINITY 1260 HPLC system equipped with Wyatt DAWN Heleos II multiangle laser light scattering detector and Wyatt OPTILAB T-rEX refractive index detector. Molar masses were calculated using dn/dc values calculated from the refractive index signal using samples with known concentration with an assumption of 100% mass recovery. Absorbance and fluorescence measurements of polymers and polyplexes were acquired using Synergy H1 multimode plate reader (BioTek; Winooski, VT). pH measurements and potentiometric titrations were done with OrionStar T910 (ThermoFisher Scientific; Waltham, MA). Flow cytometry was performed on ZE5 cell analyzer (Bio-Rad; Hercules, CA) and results were analyzed with analyzed using FlowJo software (Ashland, OR). Dynamic light scattering (DLS) measurements were made with a Zetasizer Nano ZS (Malvern; Worcestershire, UK) with a 4.0 mW He—Ne laser (λ=633 nm). Cell suspensions were counted with a Countess II automated cell counter (ThermoFisher Scientific; Waltham, MA) with dead cell discrimination by dilution (1:1) with trypan blue (0.4%). Widefield fluorescence microscopy was carried out using an EVOS Digital Microscope (AMG Life Technologies; Grand Island, NY). Confocal microscopy images were acquired on Olympus Fluoview FV1000 BX2 Upright Confocal. All statistical calculations were performed with GraphPad Prism v9.4.1.

### Methods:

Synthesis of HQ: In a round bottom flask, hydroquinine (4.0 g, 12.3 mmol) was dissolved in anhydrous tetrahydrofuran (THF; 50 mL) followed by addition of dibutyltin dilaurate (DBTDL) (23 mg, 0.036 mmol dissolved in 5 mL anhydrous THF) into the reaction mixture. A solution of 2-isocyanatoethyl acrylate (2.3 g, 16 mmol) in anhydrous THF (20 mL) was added dropwise to the reaction mixture under continuous stirring. After stirring for 30 minutes at room temperature, the reaction mixture was heated to 40° C. in an oil bath and was stirred for another 24 hours under N₂ atmosphere. Completion of the reaction was confirmed via TLC and excess 2-isocyanatoethyl acrylate was quenched by adding distilled water. The quenched reaction mixture was washed with ethyl acetate (3 times with 200 mL) to extract the product into the organic phase. The organic phase was further washed with brine, followed by drying over anhydrous Na₂SO₄ and then was concentrated under vacuum. The pure product was isolated by flash chromatography using silica gel as the stationary phase and 20% methanol in dichloromethane (DCM) as the mobile phase. The purified product was dried under high vacuum overnight before further use.

Polymerization of HQ via Reversible Addition-Fragmentation Chain-Transfer Polymerization (RAFT): Into one-dram vials, monomers (HQ and HEA), chain transfer agent (DDMAT), and initiator (AIBN) were transferred and dissolved in anhydrous dimethylformamide (DMF; total monomer concentration=1 M, [Total monomer]:[DDMAT]:[AIBN]=200:1:0.2). The vials were closed with SUBA-SEAL septa and the reaction mixture was purged with N₂ for at least 30 minutes. Subsequently, the vials were transferred to a metal heating block and stirred vigorously for 6 hours at 80° C. The reactions were quenched by rapidly cooling the reaction mixture in liquid N₂ bath followed by exposure to atmospheric oxygen. The reaction mixture was then diluted with 10% THF (inhibitor free) in methanol and then transferred to RC dialysis tubing and then dialyzed for four days using 10% THF (inhibitor free) in methanol. The purified polymer solutions were first concentrated and then dried under high vacuum overnight to yield the pure polymer. For all experiments and assays, polymer stock solutions were prepared in 3% acetic acid in water solution. The polymer solutions were vortexed well and then filtered using 0.22 micrometer syringe filter before use.

Polymerization of HQ via free radical polymerization (FRP): Into one-dram vials, HQ (with or without HEA) and AIBN were loaded and dissolved in anhydrous DMF (total monomer concentration was 1 M with [Total monomer]:[AIBN] ratio being 100:1). The vials were closed with SUBA-SEAL septa and the reaction mixture was purged with N₂ for at least 30 minutes. After that, the vials were placed in metal blocks and stirred in N₂ atmosphere at 80° C. for 15 hours. The polymerization reactions were quenched by rapidly cooling the reaction mixture in liquid N₂ baths and then exposing them to atmospheric oxygen. The reaction mixture was then diluted with 10% THF (inhibitor free) in methanol and then transferred to RC dialysis tubing and then dialyzed for four days in 10% THF (inhibitor free) in methanol. The purified polymer solutions were first concentrated and then dried under high vacuum overnight to yield the pure polymer. For all experiments and assays, the polymer stock solution was prepared in 3% acetic acid in water solution. The polymer solution was vortexed well and then filtered using a 0.22 micrometer syringe filter before use.

Potentiometric Titration: Monomers and polymers were dissolved in 30 mM HCl to prepare 15 mL of stock solutions having a polymer or monomer concentration of 1 mg/mL. This acidic solution was titrated with freshly prepared 75 mM NaOH solution, using the ORIONSTAR autotitrator, under constant stirring. The titration endpoints were determined from the derivative of the titration curve. A linear fit was applied between the titration endpoints, and the pKa values were calculated by determining the pH at the middle point of the titration endpoints. Hill coefficients were calculated by rearranging the titration curve data into Equation (1) and plotting

$$log \frac{θ}{1 - θ}$$

vs pKₐ-pH and applying linear fit.

$$log \frac{θ}{1 - θ} = {n}_{Hill} . ( p {K}_{a} - p H ) \qquad Equation ( 1 )$$

According to Equation (1), θ is the fraction of amines protonated and nail is the Hill coefficient which was determined from the slope of the

$$log \frac{θ}{1 - θ}$$

vs pKa-pH plot.

Dye Exclusion Assay: To improve the aqueous solubility of the polymers as well as to increase electrostatic interactions between the polymer chains and the pDNA, the stock solutions for the polymers were prepared in aqueous solution of acetic acid (3% glacial acetic acid in water, pH~2.6). In general, for all experiments and assays, stock solutions of polymer (0.0104 M with respect of quinuclidine nitrogen atoms) and pDNA (1 mg/mL) were diluted with ultrapure water to freshly prepare secondary solutions with appropriate concentrations. pDNA was always diluted to 0.02 mg/mL and the polymer solutions were diluted according to the desired N/P of final mixture. For the dye exclusion assay, pDNA was diluted with ultrapure water that was doped with PICOGREEN (0.5% v/v). The polymer secondary solution was added into the pDNA secondary solution in 1:1 volume ratio to form primary polyplexes for 30 minutes at room temperature. Fluorescence intensity was measured using fluorescence filter cube (λₑₓ=485/20 nm, λₑₘ=528/20 nm). The intensity from the polyplex solutions (at respective N/P ratios) without PICOGREEN was used for background subtraction. The intensity from the polymer free solutions (N/P=0) were used to normalize the intensity from the polyplex solutions. Additionally, fluorescence of the polymers in the presence of PICOGREEN but without pDNA was measured for which we only observed baseline level fluorescence signal. These controls confirmed that neither the fluorescence of the free polymer nor the interaction of PICOGREEN with polymer interferes with the dye exclusion results.

The ability of the polymers to release pDNA after formation of polyplexes were tested with a modified version of dye exclusion with the addition of heparin at a final concentration of 33.5 μg/mL. Control samples were prepared by adding water instead of heparin to the polyplex solution. The difference in normalized fluorescence intensity due to addition of heparin was used to compare the extent of pDNA release from polyplexes in the presence of heparin (FIGS. 27A-27C).

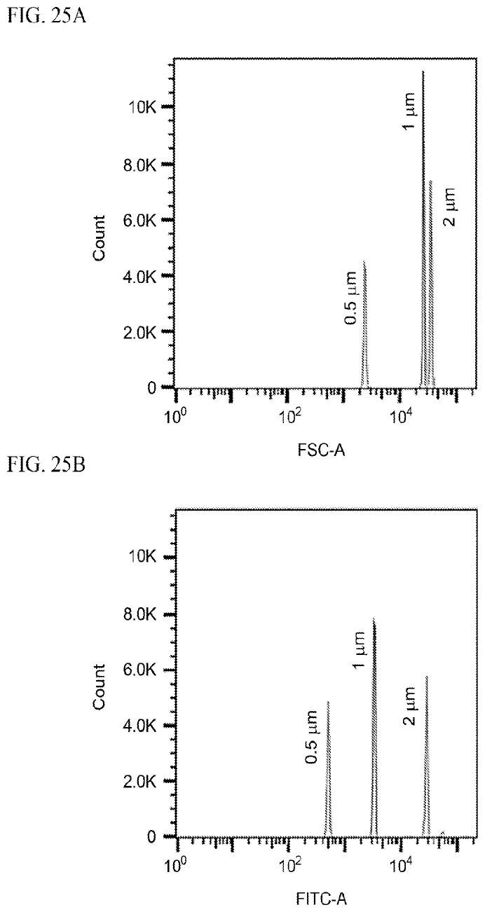

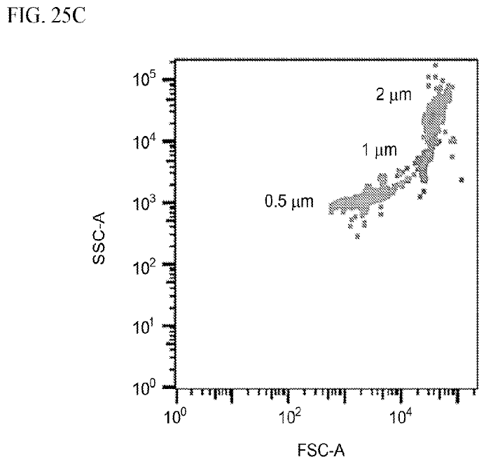

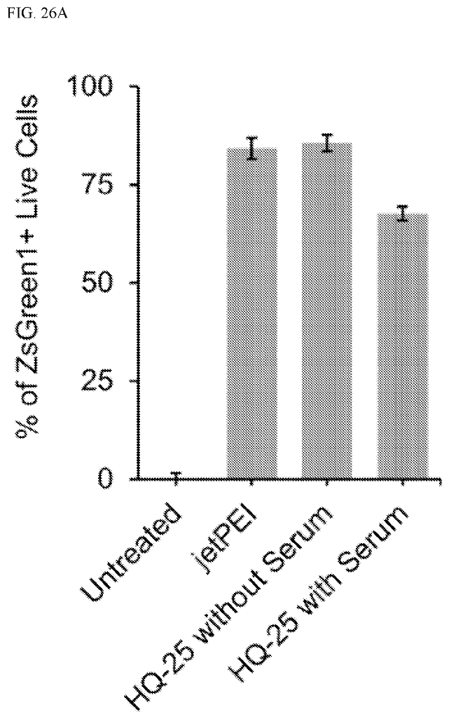

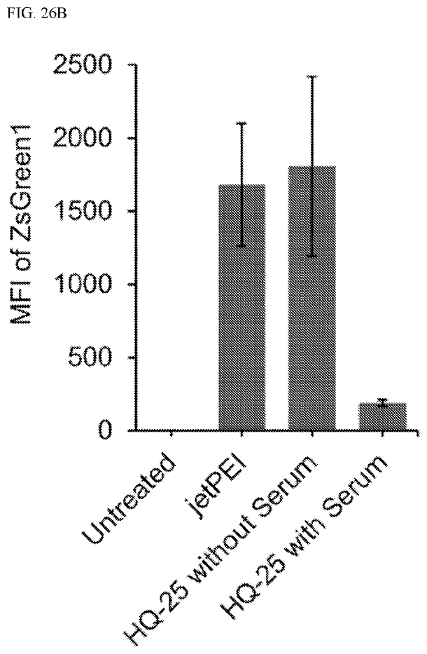

Flow Cytometry on Aggregated Polyplexes: To determine the lower size limit of detection for the polyplex particles, commercially available fluorescent particles with diameters of 100 nm, 200 nm, 500 nm, 1000 nm, and 2000 nm were used first on the flow cytometer. Forward scattering intensity and green fluorescence intensity were used in tandem to distinguish the particles from noise. From this study, the lower detection limit was found to be 500 nm in diameter (FIGS. 25A-25C21). Fluorescently labeled primary polyplex particles were formed using the general protocol described for dye exclusion assay but with the modification of using Cy5-labeled pZsGreen1-N1 pDNA. The primary polyplex solutions were diluted in 1:2 volume ratio using FLUOROBRITE DMEM to induce aggregation of the polyplexes. The aggregated polyplex solutions were split into three portions. The first portion was left untreated, the second portion was treated with 10% FBS, and the third portion was treated with FBS doped with bovine serum albumin conjugated with ALEXAFLUOR 488 (95% FBS+5% BSA-AF488). Aggregated polyplexes were also formed with unlabeled pZsGreen1-N1 pDNA to serve as controls.

Cell Culture: The engineered HEK293T and ARPE-19 cells were cultured in DMEM supplemented with 10% fetal bovine serum and 1% antibiotic-antimycotic at 37° C. and 5% CO₂ in 75 cm² and 175 cm² cell culture flasks respectively.

Transfection Protocol: Cells were plated in 24-well plates at the density of 50,000 cells/mL, 24 hours before transfection. Manufacturer's protocol was used for transfection with JETPEI and LIPOFECTAMINE 2000. For HQ-X polymers as well as QCR, previously reported transfection protocol was used with minor modifications (Van Bruggen, C., Punihaole, D.; Keith, A. R.; Schmitz, A. J.; Tolar, J.; Frontiera, R. R.; Reineke, T. M. Quinine Copolymer Reporters Promote Efficient Intracellular DNA Delivery and Illuminate a Protein-Induced Unpackaging Mechanism. Proc. Natl. Acad. Sci. U.S.A 2020, 117, 32919-32928). Similar to the protocol for the dye exclusion assay and flow cytometry on polyplexes, secondary solutions of polymer and pDNA were mixed in 1:1 volume ratio and incubated for 30 minutes at room temperature to form primary polyplexes at desired N/P ratios. The polyplex solutions were then diluted in 1:2 volume ratio using FLUOROBRITE DMEM to induce aggregation of the polyplexes. After 30 minutes, the cells were washed with phosphate buffered saline (PBS) and treated with the aggregated polyplex solution with each well receiving 1 microgram of pDNA. The cells were then incubated at 37° C. with 5% CO₂ for 30 minutes before addition of 1 mL of serum-supplemented DMEM into each well. Fresh supplemented media was added to each well 24 hours later. Negative controls were performed with cells being similarly treated with media that is equivalently diluted but does not contain any polymer and pDNA to confirm that the cells are tolerant of the media changes, the wash procedures, and the duration of transfection. Transfection in the presence of serum was also tested using HQ-25 as a model polymer, at N/P=12. To transfect the cells, aggregated polyplexes were prepared according to the regular transfection protocol followed by addition of polyplex solution directly into serum supplemented media (FIGS. 26A-26B).

Quantifying Cell Viability: Cell viability was measured using colorimetric assay with CCK-8. 48 hours after transfection, the cells were treated with a 6% solution of CCK-8 in FLUOROBRITE DMEM and incubated at 37° C. with 5% CO₂ for 1 hour. After 1 hour, the supernatant solution was transferred to a clear 96-well plate and the absorbance of the supernatant solution at 450 nm was measured. Absorbance from 6% CCK-8 solution in FLUOROBRITE DMEM was subtracted from all data points and the values were normalized to the absorbance from the supernatant of untreated cells (FIG. 17B).

Quantifying Transfection Efficiency with Flow Cytometry: 48 hours after transfection, the cells were washed with warm PBS and trypsinized using phenol red free trypsin for 5 minutes followed by quenching of trypsinization with serum supplemented phenol red free DMEM. The cell suspension was diluted with ice cold PBS containing 2% FBS and centrifuged at 4° C. at 1000 rpm for 10 minutes in deep well plates. The supernatant was discarded, and the cells were resuspended in ice cold PBS with 2% fetal bovine serum and 17 nM SYTOX Red and used for flow cytometry. Untreated cells were used as negative controls for gating. 350 nm, 488 nm, and 640 nm lasers were used for detecting HQ+, ZsGreen1+ and SYTOX Red+ cells respectively (FIG. 16A-16C). For quantifying the effect of transfection 96 hours after the transfection, a separate transfection was performed following the same protocol. 48 hours after transfection, the cells were passaged and plated into a new 24-well plates with a 1:3 split to allow continuous growth. The media was changed 24 hours later. After a total of 96 hours, the cells were prepared for flow cytometry following the protocol mentioned earlier.

Labeling pDNA with Cy5: LABEL-IT nucleic acid labeling kit from Mirus Bio (Madison, WS) was used to prepare Cy5-labeled pDNA using manufacturers protocol with one adjustment. Briefly, one full kit was used to label 1 milligram pDNA instead of 100 micro grams. The reduction in labeling density was to minimize alteration in polymer-pDNA binding while keeping sufficient number of fluorophores for detection using flow cytometry and confocal microscopy. Labeling density of the fluorescent probe was calculated using spectrophotometric method provided by Mirus Bio. The average ratio of nucleobase to Cy5 was calculated to be 440 which implies that each pDNA was labeled with 21 molecules of Cy5 on average.

Polyplex Internalization Measurement using Cy5-Labeled pDNA: Cells were transfected according to the transfection protocol using the Cy5-labeled pZsGreen plasmid. 24 hours after transfection, the cells were washed with PBS and trypsinized using phenol red free trypsin for 5 minutes followed by quenching of trypsinization with serum supplemented phenol red free media. The cell suspension was diluted with ice cold PBS containing 2% fetal bovine serum and centrifuged at 4° C. at 1000 rpm for 10 minutes in deep well plates. The supernatant was removed, and the cells were incubated with a CELLSCRUB solution for 10 minutes at room temperature followed by centrifugation at 4° C. at 1000 rpm for 10 minutes. The supernatant was discarded, and the cells were resuspended in ice cold PBS with 2% fetal bovine serum and used for flow cytometry measurements. A 640 nm laser was used for detecting Cy5+ cells and a 350 nm laser was used detecting HQ+ cells.

Confocal Microscopy Protocol: The transfection protocol was followed but the amounts of reagents were reduced by half. Fluorescent labels were carefully selected to provide sufficient spectral separation during image acquisition. Cy5-labeled pZsGreen1-N1 was used as the payload. 24 hours before transfection, 8-well chambered slides were coated with porcine gelatin solution (0.1%) followed by seeding with HEK293T cells with concentration of 25,000 cells/well. For transfection with JETPEI an N/P of 5 was used. For HQ-X polymers an N/P=16 was used. 24 hours after transfection, the cells were washed gently with PBS followed by fixation with 4% paraformaldehyde in PBS for 15 minutes at room temperature. After fixation, the fixative solution was discarded, and the cells were washed gently with PBS followed by permeabilization with washing buffer (0.1% TRITON-X100 in PBS for 5 minutes. The washing buffer was then switched with blocking buffer (5% BSA, 1% normal goat serum, 0.1% TRITON-X100 in PBS) and the cells were kept submerged in blocking buffer for 60 minutes at room temperature. Subsequently, the blocking buffer was switched with primary antibody solution (Anti-LAMP2 antibody [H4B4] diluted 1:200 in blocking buffer) and the cells were kept submerged in it overnight at 4° C. The following day, the primary antibody solution was discarded, and the cells were washed with washing buffer before treatment with a secondary antibody solution (Goat anti-mouse IgG (H+L) highly cross-adsorbed secondary antibody ALEXAFLUOR 555 diluted 1:1000 in blocking buffer) for 60 minutes. Next, the secondary antibody solution was discarded, and the cells were washed with washing buffer. Residual buffer was removed carefully, and glass a coverslip was mounted on top of the cells using SlowFade Glass (with DAPI) as the mountant and the cells were imaged on the same day. The fluorescent profile of HQ matches with DAPI (4′,6-diamidino-2-phenylindole), but the overall brightness of HQ is significantly lower than DAPI to be able to interfere in confocal imaging. Furthermore, the DAPI staining was used for cell segmentation during the image processing, and it is not connected to calculations for Pearson's correlation coefficient (PCC) which provided meaningful insight from confocal microscopy images.

All images were collected on an OLYMPUS FLUOVIEW 1000 BX2 upright confocal imaging system equipped with a PRIOR stage controller, lasers, primary dichromatic mirror, emission filters, photomultiplier tube detectors, and a PLAPON 60X oil objective (NA 1.42). Laser illumination consisted of a 405 nm and 635 nm solid state diode lasers set to 20% and 50% laser power, respectively; a 543 nm HeNe laser set to 60% laser power; and a 488 nm argon laser set to 15% laser power. The dichromatic mirrors for the various channels included a SDM490 mirror for the DAPI channel, a SDM560 mirror for the ALEXAFLUOR 488 channel, and a SDM640 mirror for the ALEXAFLUOR 555 channel. Emission bandpass filters ranged from 450/40 nm for blue emission, 515/20 nm for green emission, 610/100 nm for orange-red emission, to 705/100 nm for far red emission. Voltage settings for the photomultiplier tube detectors were 555 V with an offset of 7 for the DAPI channel, 470 V with an offset of 6 for the ALEXAFLUOR 488 channel, 705 V with an offset of 7 for the ALEXAFLUOR 555 channel, and 535 V with an offset of 4 for the Cy5 channel. Acquisition software was controlled by FLUOVIEW FV1000 software, version 4.1.1.5 (Waltham, Massachusetts).

Confocal Microscopy Image Analysis Protocol: All images were collected in Olympus Image Binary format and converted to TIFT images, which were imported into Nikon Elements Analysis software (version 5.41.01) for spectral unmixing. Labeling nuclei with DAPI inadvertently stained the Cy5-labelled pDNA as well and this had to be spectrally unmixed with Nikon Elements software using the ROI method and the unmixed images were saved as ND2 files. These files were next converted into IMS files with Imaris FileConverter (version 9.9.1) for processing, segmentation, and quantification in Imaris software (version 9.9.1). First, background was automatically subtracted from all images with a Gaussian filter radius of 17.6 to define the background at each voxel and then the software conducted a baseline subtraction of this variable background. Structures smaller than the automatically calculated filter width were removed. Second, all images were smoothed and denoised with a 3×3×3 median filter. Segmentation of nuclei and cells employed the Surfaces module. First DAPI-stained nuclei were automatically thresholded and segmented by morphological split with an estimated region growing diameter of 5.0 μm. Next, cells were detected via expression of ZsGreen1 and manually thresholded, followed by segmentation by morphological split with an estimated region growing diameter of 8.0 μm. Cy5-labeled pDNA (Cy5-pDNA) was detected and segmented with the Spots module. Region growing detection and local contrast were enabled and spot sizes were estimated as 0.6 μm laterally and 1.76 μm axially followed by manual thresholding. These surface and spot renderings were imported into the Cells module, wherein Cy5-pDNA distances to the nucleus border were measured. In Imaris software, Lysosome-Cy5-labeled pDNA colocalization was measured within the cell boundaries imposed by ZsGreen1 labeling. Lysosome and Cy5 signals were automatically thresholded as previously described (Costes, S. V.; Daelemans, D.; Cho, E. H.; Dobbin, Z.; Pavlakis, G.; Lockett, S. Automatic and Quantitative Measurement of Protein-Protein Colocalization in Live Cells. Biophys. J. 2004, 86, 3993-4003). A colocalization channel was built from which Pearson's correlation coefficients were calculated. Pearson's correlation coefficients within the colocalized volume were utilized for data analysis.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1.-27. (canceled)

28. A polymer polymerized from at least a quinoline-containing monomer of the structure 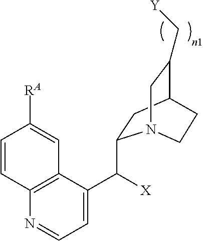
wherein:
R^{A} is H or OCH₃;
n1 is an integer from 1 to 5;
X is 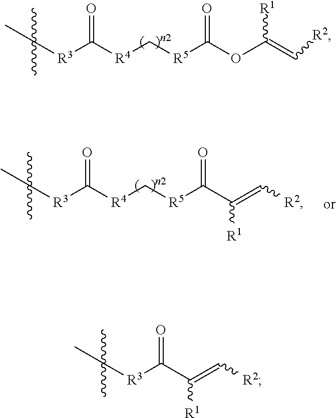
wherein:
R³, R⁴, and R⁵, are each independently O, NH, NR²⁰, or S;
wherein R²⁰ is methyl, ethyl, or propyl;
R¹ and R² are each independently H or alkyl;
n2 is an integer from 1 to 10; and
Y is alkyl.

29. The polymer of claim 28, wherein X is 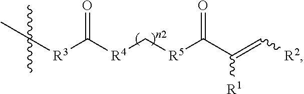
R¹ is H, R² is H, R³ is O, R⁴ is NH, R⁵ is O, and n2 is 2.

30. The polymer of claim 28, wherein R^{A} is OCH₃.

31. The polymer of claim 28, wherein Y is CH₃.

32. The polymer of claim 28, wherein the monomer is

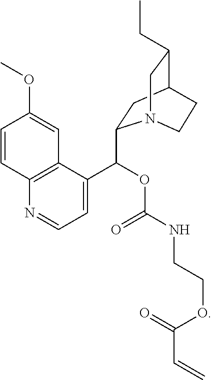 [SMILES: C=CC(=O)OCCNC(=O)OC(c1ccnc2ccc(OC)cc12)C1CC2CCN1CC2CC]

33. The polymer of claim 28, wherein the monomer is

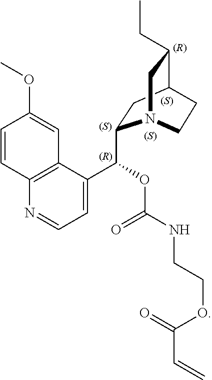

34. The polymer of claim 28, wherein the polymer is a homopolymer of the quinoline-containing monomer.

35. The polymer of claim 28, wherein the polymer is a copolymer polymerized from the quinoline-containing monomer and a second monomer.

36. The polymer of claim 35, wherein the polymer comprises 15 mol-% to 35 mol-% of the quinoline-containing monomer.

37. The copolymer of claim 35, wherein the second monomer comprises an acrylate monomer, a methacrylate monomer, an acrylamide monomer, a methacrylate monomer, or a vinyl monomer.

38. The copolymer of claim 37, wherein the acrylate monomer is (2-hydroxyethyl) acrylate or methyl acrylate.

39. A composition comprising:
a polyplex transfection complex comprising:
an oligonucleotide;
a transfection medium; and
a polymer polymerized from at least a quinoline-containing monomer of the structure 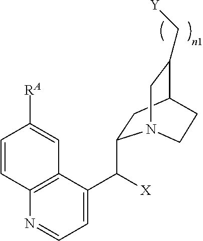
wherein:
R^{A} is H or OCH₃;
n1 is an integer from 1 to 5;
X is 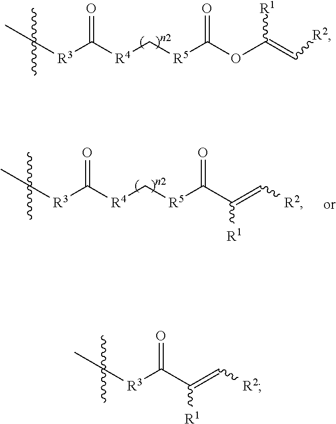
wherein:
R³, R⁴, and R⁵, are each independently O, NH, NR²⁰, or S;
wherein R²⁰ is methyl, ethyl, or propyl;
R¹ and R² are each independently H or alkyl;
n2 is an integer from 1 to 10; and
Y is alkyl.

40. The composition of claim 39, wherein the oligonucleotide comprises plasmid DNA.

41. The composition of claim 39, wherein the oligonucleotide comprises a plurality of phosphate groups, the quinoline-containing monomer comprises an amine; and a ratio of moles of the amine to moles of the plurality of phosphate groups is 2 to 20.

42. A method of forming the polyplex transfection complex of claim 39, the method comprising:
mixing the polymer with the oligonucleotide to create a first mixture; and
incubating the first mixture for a period of time to form a second mixture comprising the polyplex transfection complex.

43. The method of claim 42, where the first mixture is at a pH of 1-5.

44. The method of claim 42, further comprising diluting the second mixture with a transfection medium to form a third mixture comprising the polyplex transfection complex.

45. The method of claim 42, further comprising contacting a cell with a third mixture to form a transfection mixture; incubating the transfection mixture for a transfection time; and quenching the transfection mixture.

46. The method of claim 45, wherein the third mixture has a pH of 5 to 8.

47. The method of claim 45, wherein the transfection time is 20 minutes to 40 minutes.
